SEQUENCE LISTING (154 sequences)

SEQ ID NO: 1 - protein, 116 aa, synthetic construct
  REGION 1..116: note = Synthetic polypeptide
RVQLQQSGAELVRPGASVTLSCKASGFTFTDFEMHWVKKTPVHGLEWIGAIDPESGNTAYNQKFRGKATLTADKSSSTAYMELRYLTSEDSAVYYCTPLISGADYWGQGTTLTVSS

SEQ ID NO: 2 - protein, 112 aa, synthetic construct
  REGION 1..112: note = Synthetic polypeptide
DVVMTQSPLSLPVSLGDQASISCRSSQSLVHSDGNTYLYWYLQKPGQSPKLLIYKVSNRFSGVPDRFSGSGSGTDFTFKISRVGAEDLGVYFCSQSTHVPWTFGGGTKLEII

SEQ ID NO: 3 - protein, 120 aa, synthetic construct
  REGION 1..120: note = Synthetic polypeptide
QVQLQQPGAELVRPGSSVKLSCQASGYTFTNYWMHWVRQRPIQGLEWIGNIDPSDSETHYNQKFKDRATLTVDKSSSTAYMQLSSLTSEDSAVYYCARSGVYDDEGGDYWGQGTTLTVSS

SEQ ID NO: 4 - protein, 106 aa, synthetic construct
  REGION 1..106: note = Synthetic polypeptide
ENVLTQSPAIMSASPGEKVTMTCSASSSVSYIHWYQQKSSTSPKLWIYDTSKLASGVPGRFSGSGSGNSYSLTISSMETEDVATYYCFQGSGYPLTFGAGTKLELK

SEQ ID NO: 5 - protein, 113 aa, synthetic construct
  REGION 1..113: note = Synthetic polypeptide
EVQLQQSGPELVKPGASVKISCKASGYTFTDYYINWVRQSHGKSLECIGDINPNNGGTNYNQKFKGKATLTVDRSSNTAYMEFRSLTFDDSALYYCAAEGAYWGQGTLVTVSA

SEQ ID NO: 6 - protein, 112 aa, synthetic construct
  REGION 1..112: note = Synthetic polypeptide
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 7 - protein, 115 aa, synthetic construct
  REGION 1..115: note = Synthetic polypeptide
QVQLQQSGAELVRPGASVTLSCRASGYTFTDFEMHWVKQTPLHGLEWIGAIAPETGGTAYNQKFKGKAILTADKSSSTAYVELRSLTSEDSAVYFCTRLGGMDYWGQGTSVTVSS

SEQ ID NO: 8 - protein, 112 aa, synthetic construct
  REGION 1..112: note = Synthetic polypeptide
DVVMTQTPLSLPVSLGDQASISCRSSQTLVHNNGNTYLYWYLQKPGQSPKLLIYQVSNRFFGVPDRFSGSGSGTDFTLKISRVEAEDLGLYFCSQSTHVPWTFGGGTKLEIK

SEQ ID NO: 9 - protein, 125 aa, synthetic construct
  REGION 1..125: note = Synthetic polypeptide
QVQLVQSGAEVKKPGASVKVSCKASGYTFSRYYINWVRQAPGQGLEWMGWINPNSGGTNYAQKFQGRVTMTRDTSTSTVYMELSSLRSEDTAVYYCTTDQLRFGKTGPYYGMDVWGQGTTVTVSS

SEQ ID NO: 10 - protein, 107 aa, synthetic construct
  REGION 1..107: note = Synthetic polypeptide
DIQMTQSPSSLSASVGDRVTITCRASQNIGTYLNWYQQKPGKAPKLLIYAASSLQSGVPSRFSGSGSGTDFTLTISSLQPEDFATYYCQQSYSDTLTFGGGTKVEIK

SEQ ID NO: 11 - protein, 126 aa, synthetic construct
  REGION 1..126: note = Synthetic polypeptide
QVQLVQSGAEVKKPGASVKVSCKASGYTFTTYAVHWVRQAPGQGLEWMGIVNPNSGGTNYAQKFQGRVTMTRDTSTSTVYMELSSLRSEDTAVYYCARDIVPMNNFYNYYYGMDVWGQGTTVTVSS

SEQ ID NO: 12 - protein, 107 aa, synthetic construct
  REGION 1..107: note = Synthetic polypeptide
DIQMTQSPSSLSASVGDRVTITCRASQSISSYLNWYQQKPGKAPKLLIYATSTLQSGVPSRFSGSGSGTDFTLTISSLQPEDFATYYCQQSYRIPYTFGQGTKVEIK

SEQ ID NO: 13 - protein, 113 aa, synthetic construct
  REGION 1..113: note = Synthetic polypeptide
QVQLVQSGAEVKKPGASVKVSCKASGYTFTNYRMHWVRQAPGQGLEWMGWINPNSGGTIYAQKFQGRVTMTRDTSTSTVYMELSSLRSEDTAVYYCARDLDYWGQGTLVTVSS

SEQ ID NO: 14 - protein, 107 aa, synthetic construct
  REGION 1..107: note = Synthetic polypeptide
DIQMTQSPSSLSASVGDRVTITCQASQDISNYLNWYQQKPGKAPKLLIYGASSLQSGVPSRFSGSGSGTDFTLTISSLQPEDFATYYCQQSYSTPWTFGPGTKVEIK

SEQ ID NO: 15 - protein, 121 aa, synthetic construct
  REGION 1..121: note = Synthetic polypeptide
EVQLLESGGGLVQPGGSLRLSCAASGFTFSNAWMSWVRQAPGKGLEWVSVISGSGGRTYYADSVKGRFTISRDNSKNTLYLQMNSLRAEDTAVYYCARDGTTVAPKGMDVWGQGTTVTVSS

SEQ ID NO: 16 - protein, 107 aa, synthetic construct
  REGION 1..107: note = Synthetic polypeptide
DIQMTQSPSSLSASVGDRVTITCQASQDINNYVNWYQQKPGKAPKLLIYAASNLQSGVPSRFSGSGSGTDFTLTISSLQPEDFATYYCQQSYSPPYTFGQGTKLEIK

SEQ ID NO: 17 - protein, 126 aa, synthetic construct
  REGION 1..126: note = Synthetic polypeptide
QVQLVQSGAEVKKPGASVKVSCKASGYTFTTYAVHWVRQAPGQGLEWMGIINPNSGGTNYAQKFQGRVTMTRDTSTSTVYMELSSLRSEDTAVYYCARDIVPMNNFYNYYYGMDVWGQGTTVTVSS

SEQ ID NO: 18 - protein, 119 aa, synthetic construct
  REGION 1..119: note = Synthetic polypeptide
QVQLQQSGPELVRPGVSVKISCKGSGYTFTDYAMHWVKQSHVRSREWIGVIGTYNGHTNYNQKFKGKATMTVDKSSSTAYLELARLTSEDSAIYYCARSGSYYYSMDYWGQGTSVTVSS

SEQ ID NO: 19 - protein, 107 aa, synthetic construct
  REGION 1..107: note = Synthetic polypeptide
QIVLTQSPAIMSASPGEKVTMTCSASSSVNYMHWYQQKSGTSPKRWIYDTSKLSSGVPARFSGSGSGTSYSLTISTMEAEDAATYYCQQWSSNPPMTFGGGTKLEIK

SEQ ID NO: 20 - protein, 118 aa, synthetic construct
  REGION 1..118: note = Synthetic polypeptide
QIQLVQSGPEVKKPGETVKISCKASGYTLTDYSMHWVMQTPGTGLKWMAWMNTETGEPTYADDFKGRFAFSLETSAGTAYLQIDNLKNEDTATYFCATYGKSYGMKYWGQGTSVTVSS

SEQ ID NO: 21 - protein, 107 aa, synthetic construct
  REGION 1..107: note = Synthetic polypeptide
DIVLTQSPPSLAVSLGQRATISCRATKSVSPYLHWYQQTPGQPPKLLIYLASNLDSGVPARFSGSGSGTDFTLNIHPVEEEDAATYYCQHNKELPPTFGGGTRLEIK

SEQ ID NO: 22 - protein, 118 aa, synthetic construct
  REGION 1..118: note = Synthetic polypeptide
QIQLVQSGPELKKPGETVKISCKASGYTFTDYSMHWVRQAPGKGLKWMAWINTETGEATHADDFKGRFDFSLDTSASTAYLQIKNLKNEDTGIYFCVCYGSSYGMDYWGQGTSVTVSS

SEQ ID NO: 23 - protein, 111 aa, synthetic construct
  REGION 1..111: note = Synthetic polypeptide
DIVLTQSPASLPVSLGQRATISCRASKSVRTSGASYMHWYQQKPGQPPKLLIYLASNLKSGVPTRFSGSGSGTDFTLNIHPVEEEDAATYYCQHNREVPPTFGGGTKLEIK

SEQ ID NO: 24 - protein, 118 aa, synthetic construct
  REGION 1..118: note = Synthetic polypeptide
QIQLVQSGPELKKPGETVKISCKASGYTFTDYSMHWVKQAPGKGFKWMAWINTETGAPTHADDFRGRFAFSLETSASTAFLQISNLKTEDTATYFCVTFGKSYGMNYWGQGTSVTVSS

SEQ ID NO: 25 - protein, 111 aa, synthetic construct
  REGION 1..111: note = Synthetic polypeptide
DIVLTQSPASLSVSLGQRATISCRASKSVGSSGSSYLHWYQQKPGQPPKLLIYLASNLESGVPARFSGSGSGTEFTLNIHPVEEEDAATYYCQHNAEVPPTFGGGTKLEIK

SEQ ID NO: 26 - protein, 114 aa, synthetic construct
  REGION 1..114: note = Synthetic polypeptide
QIQLVQSGPELKKPGETVKISCKASGYTFTDYSLHWVKQTPEKNLKWMAWINTETGAPTYADDFKGRFAFSLETSSNTAYLLINNLKNEDTATYFCALTMVNYWGQGTTLTVSS

SEQ ID NO: 27 - protein, 107 aa, synthetic construct
  REGION 1..107: note = Synthetic polypeptide
DIVMTQSQKFMSTSVGDRVSVTCKASQNVGIRVAWHQQKPGQSPKTLIYSTSYRYSGVPDRFTGSGSGTEFILTISNVQSEDLAEYFCQQYNSYPYTFGGGTKLEIK

SEQ ID NO: 28 - protein, 118 aa, synthetic construct
  REGION 1..118: note = Synthetic polypeptide
QIQLVQSGPELKKPGETVKISCQVSGYTFTDYSMHWLKQAPGKGLKWMAWINTETGESTYTDEFKGRFVFSLETSVRTAYLKINNLKNEDTATYFCGTYGSSYGMAYWGQGTSVTVSS

SEQ ID NO: 29 - protein, 111 aa, synthetic construct
  REGION 1..111: note = Synthetic polypeptide
DIVLTQSPASLPVSLGQRATISCRASKSVSTSGSSYLHWYQQKPGQPPKLLIYLASKLESGVPARFRGSGSGTDFTLNIHPVEEEDAATYYCQHNREVPPTFGGGTKLEIK

SEQ ID NO: 30 - protein, 118 aa, synthetic construct
  REGION 1..118: note = Synthetic polypeptide
QIQLVQSGPELKKPGETVKISCKASGYTFTDYSMHWVKQAPGKGLKWMAWINTETGEPTYADDFRGRFAFSLETSGRTAYLEINNLRNEDTATYFCVTYGSSYGMAYWGQGTSVTVSS

SEQ ID NO: 31 - protein, 111 aa, synthetic construct
  REGION 1..111: note = Synthetic polypeptide
DIVLTQSPASLAVSLGQRATISCRASKSVSRSGYSYLHWYQQKPGQPPKLLIYLASKLESGVPARFSGSGSGTDFTLNIHPVEAEDAATYYCQHNREIPPTFGGGTKLEIK

SEQ ID NO: 32 - protein, 118 aa, synthetic construct
  REGION 1..118: note = Synthetic polypeptide
QIQLVQSGPELKKPGETVKISCKASGYTLTDYSMHWVKQAPGKGLKWMGWINTETGEPRYGDDFKGRFAFSLETSASSAYLQINNLKNDDTATYFCATYGRSYGMDFWGQGTSVTVSS

SEQ ID NO: 33 - protein, 111 aa, synthetic construct
  REGION 1..111: note = Synthetic polypeptide
DIVLTQSPASLAVSLGQRATISCRASKSVSPSGHSYMHWYQQKPGQPPKLLIYLASNLKSGVPARFSGSGSGTDFTLNIHPVEEGDAATYYCQHNRELPPTFGGGSKLEIK

SEQ ID NO: 34 - protein, 114 aa, synthetic construct
  REGION 1..114: note = Synthetic polypeptide
DVQLQESGPDLVKPSQSLSLTCTVTGYSITSGYSWHWIRQFPDNKLEWMGYILSRGDTNFNPSLKSRISITRSTSNNQFFLHFNSVTTEDTATYYCARDFFDYWGQGTTLTVSS

SEQ ID NO: 35 - protein, 112 aa, synthetic construct
  REGION 1..112: note = Synthetic polypeptide
DVLMTQTPLSLPVSLGDQASISCRSSQSIVHSNGNTYLEWYLQKPGQSPKLLIYKVSDRFSGVPDRFSGSGSGTDFTLKISRVEAEDLGVYFCFQGSHVPYTFGGGTKLEIK

SEQ ID NO: 36 - protein, 5 aa, synthetic construct
  REGION 1..5: note = Synthetic peptide
DFEMH

SEQ ID NO: 37 - protein, 5 aa, synthetic construct
  REGION 1..5: note = Synthetic peptide
NYWMH

SEQ ID NO: 38 - protein, 5 aa, synthetic construct
  REGION 1..5: note = Synthetic peptide
DYYIN

SEQ ID NO: 39 - protein, 5 aa, synthetic construct
  REGION 1..5: note = Synthetic peptide
RYYIN

SEQ ID NO: 40 - protein, 5 aa, synthetic construct
  REGION 1..5: note = Synthetic peptide
TYAVH

SEQ ID NO: 41 - protein, 5 aa, synthetic construct
  REGION 1..5: note = Synthetic peptide
NYRMH

SEQ ID NO: 42 - protein, 5 aa, synthetic construct
  REGION 1..5: note = Synthetic peptide
NAWMS

SEQ ID NO: 43 - protein, 5 aa, synthetic construct
  REGION 1..5: note = Synthetic peptide
DYAMH

SEQ ID NO: 44 - protein, 5 aa, synthetic construct
  REGION 1..5: note = Synthetic peptide
DYSMH

SEQ ID NO: 45 - protein, 5 aa, synthetic construct
  REGION 1..5: note = Synthetic peptide
DYSLH

SEQ ID NO: 46 - protein, 6 aa, synthetic construct
  REGION 1..6: note = Synthetic peptide
SGYSWH

SEQ ID NO: 47 - protein, 17 aa, synthetic construct
  REGION 1..17: note = Synthetic peptide
AIDPESGNTAYNQKFRG

SEQ ID NO: 48 - protein, 17 aa, synthetic construct
  REGION 1..17: note = Synthetic peptide
NIDPSDSETHYNQKFKD

SEQ ID NO: 49 - protein, 17 aa, synthetic construct
  REGION 1..17: note = Synthetic peptide
DINPNNGGTNYNQKFKG

SEQ ID NO: 50 - protein, 17 aa, synthetic construct
  REGION 1..17: note = Synthetic peptide
AIAPETGGTAYNQKFKG

SEQ ID NO: 51 - protein, 17 aa, synthetic construct
  REGION 1..17: note = Synthetic peptide
WINPNSGGTNYAQKFQG

SEQ ID NO: 52 - protein, 17 aa, synthetic construct
  REGION 1..17: note = Synthetic peptide
IVNPNSGGTNYAQKFQG

SEQ ID NO: 53 - protein, 17 aa, synthetic construct
  REGION 1..17: note = Synthetic peptide
WINPNSGGTIYAQKFQG

SEQ ID NO: 54 - protein, 17 aa, synthetic construct
  REGION 1..17: note = Synthetic peptide
VISGSGGRTYYADSVKG

SEQ ID NO: 55 - protein, 17 aa, synthetic construct
  REGION 1..17: note = Synthetic peptide
IINPNSGGTNYAQKFQG

SEQ ID NO: 56 - protein, 17 aa, synthetic construct
  REGION 1..17: note = Synthetic peptide
VIGTYNGHTNYNQKFKG

SEQ ID NO: 57 - protein, 17 aa, synthetic construct
  REGION 1..17: note = Synthetic peptide
WMNTETGEPTYADDFKG

SEQ ID NO: 58 - protein, 17 aa, synthetic construct
  REGION 1..17: note = Synthetic peptide
WINTETGEATHADDFKG

SEQ ID NO: 59 - protein, 17 aa, synthetic construct
  REGION 1..17: note = Synthetic peptide
WINTETGAPTHADDFRG

SEQ ID NO: 60 - protein, 17 aa, synthetic construct
  REGION 1..17: note = Synthetic peptide
WINTETGAPTYADDFKG

SEQ ID NO: 61 - protein, 17 aa, synthetic construct
  REGION 1..17: note = Synthetic peptide
WINTETGESTYTDEFKG

SEQ ID NO: 62 - protein, 17 aa, synthetic construct
  REGION 1..17: note = Synthetic peptide
WINTETGEPTYADDFRG

SEQ ID NO: 63 - protein, 17 aa, synthetic construct
  REGION 1..17: note = Synthetic peptide
WINTETGEPRYGDDFKG

SEQ ID NO: 64 - protein, 16 aa, synthetic construct
  REGION 1..16: note = Synthetic peptide
YILSRGDTNFNPSLKS

SEQ ID NO: 65 - protein, 7 aa, synthetic construct
  REGION 1..7: note = Synthetic peptide
LISGADY

SEQ ID NO: 66 - protein, 11 aa, synthetic construct
  REGION 1..11: note = Synthetic peptide
SGVYDDEGGDY

SEQ ID NO: 67 - protein, 4 aa, synthetic construct
  REGION 1..4: note = Synthetic peptide
EGAY

SEQ ID NO: 68 - protein, 6 aa, synthetic construct
  REGION 1..6: note = Synthetic peptide
LGGMDY

SEQ ID NO: 69 - protein, 16 aa, synthetic construct
  REGION 1..16: note = Synthetic peptide
DQLRFGKTGPYYGMDV

SEQ ID NO: 70 - protein, 17 aa, synthetic construct
  REGION 1..17: note = Synthetic peptide
DIVPMNNFYNYYYGMDV

SEQ ID NO: 71 - protein, 4 aa, synthetic construct
  REGION 1..4: note = Synthetic peptide
DLDY

SEQ ID NO: 72 - protein, 12 aa, synthetic construct
  REGION 1..12: note = Synthetic peptide
DGTTVAPKGMDV

SEQ ID NO: 73 - protein, 10 aa, synthetic construct
  REGION 1..10: note = Synthetic peptide
SGSYYYSMDY

SEQ ID NO: 74 - protein, 9 aa, synthetic construct
  REGION 1..9: note = Synthetic peptide
YGKSYGMKY

SEQ ID NO: 75 - protein, 9 aa, synthetic construct
  REGION 1..9: note = Synthetic peptide
FGKSYGMNY

SEQ ID NO: 76 - protein, 5 aa, synthetic construct
  REGION 1..5: note = Synthetic peptide
TMVNY

SEQ ID NO: 77 - protein, 9 aa, synthetic construct
  REGION 1..9: note = Synthetic peptide
YGSSYGMAY

SEQ ID NO: 78 - protein, 9 aa, synthetic construct
  REGION 1..9: note = Synthetic peptide
YGRSYGMDF

SEQ ID NO: 79 - protein, 5 aa, synthetic construct
  REGION 1..5: note = Synthetic peptide
DFFDY

SEQ ID NO: 80 - protein, 16 aa, synthetic construct
  REGION 1..16: note = Synthetic peptide
RSSQSLVHSDGNTYLY

SEQ ID NO: 81 - protein, 10 aa, synthetic construct
  REGION 1..10: note = Synthetic peptide
SASSSVSYIH

SEQ ID NO: 82 - protein, 16 aa, synthetic construct
  REGION 1..16: note = Synthetic peptide
KSSQSLLDSDGKTYLN

SEQ ID NO: 83 - protein, 16 aa, synthetic construct
  REGION 1..16: note = Synthetic peptide
RSSQTLVHNNGNTYLY

SEQ ID NO: 84 - protein, 11 aa, synthetic construct
  REGION 1..11: note = Synthetic peptide
RASQNIGTYLN

SEQ ID NO: 85 - protein, 11 aa, synthetic construct
  REGION 1..11: note = Synthetic peptide
RASQSISSYLN

SEQ ID NO: 86 - protein, 11 aa, synthetic construct
  REGION 1..11: note = Synthetic peptide
QASQDISNYLN

SEQ ID NO: 87 - protein, 11 aa, synthetic construct
  REGION 1..11: note = Synthetic peptide
QASQDINNYVN

SEQ ID NO: 88 - protein, 10 aa, synthetic construct
  REGION 1..10: note = Synthetic peptide
SASSSVNYMH

SEQ ID NO: 89 - protein, 11 aa, synthetic construct
  REGION 1..11: note = Synthetic peptide
RATKSVSPYLH

SEQ ID NO: 90 - protein, 15 aa, synthetic construct
  REGION 1..15: note = Synthetic peptide
RASKSVRTSGASYMH

SEQ ID NO: 91 - protein, 15 aa, synthetic construct
  REGION 1..15: note = Synthetic peptide
RASKSVGSSGSSYLH

SEQ ID NO: 92 - protein, 11 aa, synthetic construct
  REGION 1..11: note = Synthetic peptide
KASQNVGIRVA

SEQ ID NO: 93 - protein, 15 aa, synthetic construct
  REGION 1..15: note = Synthetic peptide
RASKSVSTSGSSYLH

SEQ ID NO: 94 - protein, 15 aa, synthetic construct
  REGION 1..15: note = Synthetic peptide
RASKSVSRSGYSYLH

SEQ ID NO: 95 - protein, 15 aa, synthetic construct
  REGION 1..15: note = Synthetic peptide
RASKSVSPSGHSYMH

SEQ ID NO: 96 - protein, 16 aa, synthetic construct
  REGION 1..16: note = Synthetic peptide
RSSQSIVHSNGNTYLE

SEQ ID NO: 97 - protein, 7 aa, synthetic construct
  REGION 1..7: note = Synthetic peptide
KVSNRFS

SEQ ID NO: 98 - protein, 7 aa, synthetic construct
  REGION 1..7: note = Synthetic peptide
DTSKLAS

SEQ ID NO: 99 - protein, 7 aa, synthetic construct
  REGION 1..7: note = Synthetic peptide
LVSKLDS

SEQ ID NO: 100 - protein, 7 aa, synthetic construct
  REGION 1..7: note = Synthetic peptide
QVSNRFF

SEQ ID NO: 101 - protein, 7 aa, synthetic construct
  REGION 1..7: note = Synthetic peptide
AASSLQS

SEQ ID NO: 102 - protein, 7 aa, synthetic construct
  REGION 1..7: note = Synthetic peptide
ATSTLQS

SEQ ID NO: 103 - protein, 7 aa, synthetic construct
  REGION 1..7: note = Synthetic peptide
GASSLQS

SEQ ID NO: 104 - protein, 7 aa, synthetic construct
  REGION 1..7: note = Synthetic peptide
AASNLQS

SEQ ID NO: 105 - protein, 7 aa, synthetic construct
  REGION 1..7: note = Synthetic peptide
DTSKLSS

SEQ ID NO: 106 - protein, 7 aa, synthetic construct
  REGION 1..7: note = Synthetic peptide
LASNLDS

SEQ ID NO: 107 - protein, 7 aa, synthetic construct
  REGION 1..7: note = Synthetic peptide
LASNLKS

SEQ ID NO: 108 - protein, 7 aa, synthetic construct
  REGION 1..7: note = Synthetic peptide
LASNLES

SEQ ID NO: 109 - protein, 7 aa, synthetic construct
  REGION 1..7: note = Synthetic peptide
STSYRYS

SEQ ID NO: 110 - protein, 7 aa, synthetic construct
  REGION 1..7: note = Synthetic peptide
LASKLES

SEQ ID NO: 111 - protein, 7 aa, synthetic construct
  REGION 1..7: note = Synthetic peptide
KVSDRFS

SEQ ID NO: 112 - protein, 9 aa, synthetic construct
  REGION 1..9: note = Synthetic peptide
SQSTHVPWT

SEQ ID NO: 113 - protein, 9 aa, synthetic construct
  REGION 1..9: note = Synthetic peptide
FQGSGYPLT

SEQ ID NO: 114 - protein, 9 aa, synthetic construct
  REGION 1..9: note = Synthetic peptide
WQGTHFPWT

SEQ ID NO: 115 - protein, 9 aa, synthetic construct
  REGION 1..9: note = Synthetic peptide
QQSYSDTLT

SEQ ID NO: 116 - protein, 9 aa, synthetic construct
  REGION 1..9: note = Synthetic peptide
QQSYRIPYT

SEQ ID NO: 117 - protein, 9 aa, synthetic construct
  REGION 1..9: note = Synthetic peptide
QQSYSTPWT

SEQ ID NO: 118 - protein, 9 aa, synthetic construct
  REGION 1..9: note = Synthetic peptide
QQSYSPPYT

SEQ ID NO: 119 - protein, 10 aa, synthetic construct
  REGION 1..10: note = Synthetic peptide
QQWSSNPPMT

SEQ ID NO: 120 - protein, 9 aa, synthetic construct
  REGION 1..9: note = Synthetic peptide
QHNKELPPT

SEQ ID NO: 121 - protein, 9 aa, synthetic construct
  REGION 1..9: note = Synthetic peptide
QHNREVPPT

SEQ ID NO: 122 - protein, 9 aa, synthetic construct
  REGION 1..9: note = Synthetic peptide
QHNAEVPPT

SEQ ID NO: 123 - protein, 9 aa, synthetic construct
  REGION 1..9: note = Synthetic peptide
QQYNSYPYT

SEQ ID NO: 124 - protein, 9 aa, synthetic construct
  REGION 1..9: note = Synthetic peptide
QHNREIPPT

SEQ ID NO: 125 - protein, 9 aa, synthetic construct
  REGION 1..9: note = Synthetic peptide
QHNRELPPT

SEQ ID NO: 126 - protein, 9 aa, synthetic construct
  REGION 1..9: note = Synthetic peptide
FQGSHVPYT

SEQ ID NO: 127 - protein, 201 aa, Homo sapiens
MWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHYMLLVFVKVPILLILVTAILWLKGSQRVPEEPGEQPIYMNFSEPLTKDMAT

SEQ ID NO: 128 - protein, 209 aa, Mus musculus
MWLSPALLLLSFPGCLSIQGPALVRGPEQGSVTVQCRYSSRWQTNKKWWCRGASWSTCRVLIRSTGSEKETKSGRLSIRDNQKNHSFQVTMEMLRQNDTDTYWCGIEKFGTDRGTRVKVNVYSVGKDTMSTSNQLPWPTVDGSTDMVSSDLQKRTYYMLLVFVKVPALLILVGAVLWLKRSTQKVPEEQWRHTLCSDLDSELLAKDISP

SEQ ID NO: 129 - protein, 201 aa, Macaca mulatta
MWLSPALLLLSLSGCFSIRGPVSVRGPEKGSVKVQCHYKRGWETHVKWWCRGARWVSCEILIQTRGSEQEEKKDLVSIKDSHTDCTFTVTMEGLRQNDSNIYWCGIQRRGPDLGTQVQLTVDPEGAASAPANSSVNSNIRVSISTHKRNHYMLLVFVKVPILLILVGAILWLKGFQRVPEEPGDQPIYMNFSELLTKDMAA

SEQ ID NO: 130 - protein, 151 aa, Homo sapiens
MWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHY

SEQ ID NO: 131 - protein, 155 aa, Mus musculus
MWLSPALLLLSFPGCLSIQGPALVRGPEQGSVTVQCRYSSRWQTNKKWWCRGASWSTCRVLIRSTGSEKETKSGRLSIRDNQKNHSFQVTMEMLRQNDTDTYWCGIEKFGTDRGTRVKVNVYSVGKDTMSTSNQLPWPTVDGSTDMVSSDLQKRT

SEQ ID NO: 132 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 133 - protein, 235 aa, Homo sapiens
MWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNQAQSDCSCSTVSPGVLAGIVMGALVLTVLIALAVYFLGRLVPRGRGAAEAATRKQRITETESPYQELQGQRSDVYSDLNTQRPYYK

SEQ ID NO: 134 - protein, 242 aa, Mus musculus
MWLSPALLLLSFPGCLSIQGPALVRGPEQGSVTVQCRYSSRWQTNKKWWCRGASWSTCRVLIRSTGSEKETKSGRLSIRDNQKNHSFQVTMEMLRQNDTDTYWCGIEKFGTDRGTRVKVNVYSVGKDTMSTSNQLPWPTVDGSTDMVSSDLQKRTQSDTFPRCDCSSVSPGVLAGIVLGALVLTLLIALAVYSLGRLVSRGQGTAEGTRKQHIAETESPYQELQGQRPEVYSDLNTQRQYYR

SEQ ID NO: 135 - protein, 311 aa, Homo sapiens
MGWSCIILFLVATATGVHSEQKLISEEDLLSKCRTVAGPVGGSLSVQCPYEKEHRTLNKYWCRPPQIFLCDKIVETKGSAGKRNGRVSIRDSPANLSFTVTLENLTEEDAGTYWCGVDTPWLRDFHDPVVEVEVSVFPASTSMTPASITAAKTSTITTAFPPVSSTTLFAVGATHSASIQEETEEVVNSQLPLLLSLLALLLLLLVGASLLAWRMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 136 - protein, 270 aa, Homo sapiens
MGWSCIILFLVATATGVHSEQKLISEEDLMTVAGPVGGSLSVQCRYEKEHRTLNKFWCRPPQILRCDKIVETKGSAGKRNGRVSIRDSPANLSFTVTLENLTEEDAGTYWCGVDTPWLRDFHDPIVEVEVSVFPAGTTTASSPQSSMGTSGPPTKLPVHTWPSVTRKDSPEPSPHPGSLFSNVRQAQSDCSCSTVSPGVLAGIVMGALVLTVLIALAVYFLGRLVPRGRGAAEAATRKQRITETESPYQELQGQRSDVYSDLNTQRPYYK

SEQ ID NO: 137 - protein, 262 aa, Homo sapiens
MGWSCIILFLVATATGVHSEQKLISEEDLAKITGPTTVNGSEQGSLTVQCAYGSGWETYLKWRCQGADWNYCNILVKTNGSEQEVKKNRVSIRDNQKNHVFTVTMENLKRDDADSYWCGTERPGIDLGVKVQVTINPGTQTAVSEWTTTTASLAFTAAATQKTSSPLTRSPLKSTHQAQSDCSCSTVSPGVLAGIVMGALVLTVLIALAVYFLGRLVPRGRGAAEAATRKQRITETESPYQELQGQRSDVYSDLNTQRPYYK

SEQ ID NO: 138 - protein, 231 aa, Homo sapiens
MGWSCIILFLVATATGVHSEQKLISEEDLLKGPGSVTGTAGDSLTVWCQYESMYKGYNKYWCRGQYDTSCESIVETKGEEKVERNGRVSIRDHPEALAFTVTMQNLNEDDAGSYWCKIQTVWVLDSWSRDPSDLVRVYVSPAITTPRRTTHPATPPIFLVVNPGRNLSTGEVLTQNSGFRLSSPHQAQSDCSCSTVSPGVLAGIVMGALVLTVLIALAVYFLGRLVPRGRG

SEQ ID NO: 139 - protein, 300 aa, Homo sapiens
MGWSCIILFLVATATGVHSEQKLISEEDLTQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSSPTLTGHHLDNRHKLLKLSVLLPLIFTILLLLLVAASLLAWRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 140 - protein, 343 aa, Homo sapiens
MGWSCIILFLVATATGVHSEQKLISEEDLLEGPEEISGFEGDTVSLQCTYREELRDHRKYWCRKGGILFSRCSGTIYAEEEGQETMKGRVSIRDSRQELSLIVTLWNLTLQDAGEYWCGVEKRGPDESLLISLFVFPGPCCPPSPSPTFQPLATTRLQPKAKAQQTQPPGLTSPGLYPAATTAKQGKTGAEAPPLPGTSQYGHERTSQYTGTSPHPATSPPAGSSRPPMQLDSTSAEDTSPALSSGSSKPRVSIPMVRILAPVLVLLSLLSAAGLIAFCSHLLLWRKEAQQATETQRNEKFCLSRLTAEEKEAPSQAPEGDVISMPPLHTSEEELGFSKFVSA

SEQ ID NO: 141 - protein, 318 aa, Mus musculus
MTQLASAVWLPTLLLLLLLFWLPGCVPLHGPSTMSGSVGESLSVSCRYEEKFKTKDKYWCRVSLKILCKDIVKTSSSEEARSGRVTIRDHPDNLTFTVTYESLTLEDADTYMCAVDISLFDGSLGFDKYFKIELSVVPSEDPVSSPGPTLETPVVSTSLPTKGPALGSNTEGHREHDYSQGLRLPALLSVLALLLFLLVGTSLLAWRMFQKRLVKADRHPELSQNLRQASEQNECQYVNLQLHTWSLREEPVLPSQVEVVEYSTLALPQEELHYSSVAFNSQRQDSHANGDSLHQPQDQKAEYSEIQKPRKGLSDLYL

SEQ ID NO: 142 - protein, 228 aa, Mus musculus
MIPRVIRLWLPSALFLSQVPGCVPLHGPSTITGAVGESLSVSCQYEEKFKTKDKFWCRGSLKVLCKDIVKTSSSEEVRNGRVTIRDHPDNLTFTVTYESLTLEDADTYMCAVDISLFDGSLGFDKYFKIELSVVPSEDPVTGSSLESGRDILESPTSSVGHTHPSVTTDDTIPAPCPQPRSLRSSLYFWVLVSLKLFLFLSMLGAVLWVNRPQRCSGGSSSRPCYENQ

SEQ ID NO: 143 - protein, 337 aa, Mus musculus
MHLSLLVPFLFWITGCCTAEDPVTGPEEVSGQEQGSLTVQCRYTSGWKDYKKYWCQGVPQRSCKTLVETDASEQLVKKNRVSIRDNQRDFIFTVTMEDLRMSDAGIYWCGITKGGLDPMFKVTVNIGPAIQVPITVPTMPPITSTTTIFTVTTTVKETSMFPTLTSYYSDNGHGGGDSGGGEDGVGDGFLDLSVLLPVISAVLLLLLLVASLFAWRMVRRQKKAAGPPSEQAQSLEGDLCYADLSLKQPRTSPGSSWKKGSSMSSSGKDHQEEVEYVTMAPFPREEVSYAALTLAGLGQEPTYGNTGCPITHVPRTGLEEETTEYSSIRRPLPAAMP

SEQ ID NO: 144 - protein, 398 aa, Homo sapiens
LSKCRTVAGPVGGSLSVQCPYEKEHRTLNKYWCRPPQIFLCDKIVETKGSAGKRNGRVSIRDSPANLSFTVTLENLTEEDAGTYWCGVDTPWLRDFHDPVVEVEVSVFPASTSMTPASITAAKTSTITTAFPPVSSTTLFAVGATHSASIQEETEEVVNSQIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 145 - protein, 370 aa, Homo sapiens
IQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 146 - protein, 392 aa, Homo sapiens
MTVAGPVGGSLSVQCRYEKEHRTLNKFWCRPPQILRCDKIVETKGSAGKRNGRVSIRDSPANLSFTVTLENLTEEDAGTYWCGVDTPWLRDFHDPIVEVEVSVFPAGTTTASSPQSSMGTSGPPTKLPVHTWPSVTRKDSPEPSPHPGSLFSNVRIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 147 - protein, 384 aa, Homo sapiens
AKITGPTTVNGSEQGSLTVQCAYGSGWETYLKWRCQGADWNYCNILVKTNGSEQEVKKNRVSIRDNQKNHVFTVTMENLKRDDADSYWCGTERPGIDLGVKVQVTINPGTQTAVSEWTTTTASLAFTAAATQKTSSPLTRSPLKSTHIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 148 - protein, 389 aa, Homo sapiens
LKGPGSVTGTAGDSLTVWCQYESMYKGYNKYWCRGQYDTSCESIVETKGEEKVERNGRVSIRDHPEALAFTVTMQNLNEDDAGSYWCKIQTVWVLDSWSRDPSDLVRVYVSPAITTPRRTTHPATPPIFLVVNPGRNLSTGEVLTQNSGFRLIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 149 - protein, 377 aa, Homo sapiens
YSIATQITGPTTVNGLERGSLTVQCVYRSGWETYLKWWCRGAIWRDCKILVKTSGSEQEVKRDRVSIKDNQKNRTFTVTMEDLMKTDADTYWCGIEKTGNDLGVTVQVTIDPAPVTQEETSSSPTLTGHHLDNRHKLLKLIEGRMDPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 150 - protein, 393 aa, Mus musculus
LHGPSTMTGSVGQSLSVSCQYEEKFKTKDKYWCRGSLKVLCKDIVKTSSSEEARSGRVTIRDHPDNLTFTVTYESLTLDDADTYMCAVDIPFFNAPLGLDKYFKIELSVVPSEDPVSSPGPTLETPVVSTSLPTKGPALGSNTEDRREHDYSQGLRIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 380 aa, Mus musculus
IQGPALVRGPEQGSVTVQCRYSSRWQTNKKWWCRGASWSTCRVLIRSTGSEKETKSGRLSIRDNQKNHSFQVTMEMLRQNDTDTYWCGIEKFGTDRGTRVKVNVYSVGKDTMSTSNQLPWPTVDGSTDMVSSDLQKRTYYIEGRMDPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 152 - protein, 406 aa, Mus musculus
HFPVRGPSTVTGTVGESLSVSCQYEKKLKTKKKIWCKWKSNVLCKDIVKTSASEEARNGRVSIRDHPDNLTFTVTLENLTLEDAGTYMCMVDIGFFYDAYLQIDKSFKVEVFVVPGKPPFKGSRPGNGINILASPTSSAVHTQPNVTTDDTIPAPSPELRSLLSSPIEGRMDPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 153 - protein, 393 aa, Mus musculus
NPVTGPEEVSGQEQGSLTVQCQYTSDWKDYKKYWCQGVPQKSCVFLIETDKSEQLVKKNRVSIRDNQREFIFTVIMEDLRMSDAGIYWCGITKAGYDPVFKVNVSINPAPKSSMMTTTATVLKSIQPSAENTGKEQVTQSKEVTQSRPHTRSLIEGRMDPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 154 - protein, 389 aa, Mus musculus
LTGPGSVSGYVGGSLRVQCQYSPSYKGYMKYWCRGPHDTTCKTIVETDGSEKEKRSGPVSIRDHAANSTITVIMEDLSEDDAGSYWCKIQTSFIWDSWSRDPSVSVRVNVFPATTPTLPATTAILPLVNSGQNLRISTNVMFIFQLWSLIEGRMDPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2024/0254227 A1
(43) Pub. Date: Aug. 1, 2024

(54) Anti-CD300LB Antibodies and Methods of Use Thereof

(71) Applicant: Alector LLC, South San Francisco, CA (US)

(72) Inventors: Sarah Emily Headland, Alameda, CA (US); Ilaria Tassi, San Francisco, CA (US); Chenyu A. Lee, San Diego, CA (US); Earl Kim, San Diego, CA (US); Adiljan Ibrahim, San Ramon, CA (US); Marina Roell, Concord, CA (US); Angie Grace Yee, San Francisco, CA (US)

(73) Assignee: Alector LLC, South San Francisco, CA (US)

(21) Appl. No.: 18/637,960

(22) Filed: Apr. 17, 2024

Related U.S. Application Data
(63) Continuation of application No. PCT/US2022/078256, filed on Oct. 18, 2022.
(60) Provisional application No. 63/257,349, filed on Oct. 19, 2021.

(51) Int. Cl. C07K 16/28; A61K 39/00

(52) U.S. Cl. CPC C07K 16/2803; A61K 2039/505; C07K 2317/24; C07K 2317/31; C07K 2317/33; C07K 2317/56; C07K 2317/92

(57) ABSTRACT

The present disclosure is generally directed to antibodies, e.g., monoclonal, antibodies, antibody fragments, etc., that specifically bind a CD300LB polypeptide, e.g., a mammalian CD300LB or human CD300LB, and use of such compositions in preventing, reducing risk, or treating a disease or disorder an individual in need thereof.

### CROSS-REFERENCE TO RELATED APPLICATIONS

This application is a continuation of International Application No. PCT/US2022/078256, filed on Oct. 18, 2022, which claims the benefit of priority to U.S. Provisional Application No. 63/257,349, filed Oct. 19, 2021, the contents of both of which are incorporated herein by reference for all purposes.

### SUBMISSION OF SEQUENCE LISTING ON ASCII TEXT FILE

The instant application contains a Sequence Listing which has been submitted electronically in XML format and is hereby incorporated by reference in its entirety. Said XML copy, created on Sep. 14, 2022, is named 2022-09-14_01209-0012-00PCT_Sequence-Listing and is 196,608 bytes in size.

### FIELD OF THE PRESENT DISCLOSURE

The present disclosure relates to anti-CD300LB antibodies and uses (e.g., therapeutic uses) of such antibodies.

### BACKGROUND OF THE PRESENT DISCLOSURE

The CD300 gene family, located on human chromosome 17, is a class of type II transmembrane receptors with roles in immunity and inflammation. Of the human CD300 family, of which there are 8 members (CD300A-H) described in the literature, only the CD300LB gene has been genetically linked to Alzheimer's disease. CD300LB is highly enriched in microglia compared to other brain tissues in humans (>30×, p=E-18) and is among a set of 50 microglial transmembrane genes with common genetic variants showing suggestive GW-association in IGAP GWAS (International Genomics of Alzheimer's Project) and the association with Alzheimer's Disease (AD) is independently replicated in the UK Biobank: a very rare premature stop codon (rs139180365, Arg88 stop, MAF=0.02%) is associated with AD parental cases (OR: 1.8, p=1E−4). CD300LB contains an immunoreceptor tyrosine-based activation motif, and couples with the DAP12 signaling molecule to transduce signals via the Syk signaling pathway. CD300LB is an activating (ITAM) receptor on microglia that binds lipids (phosphatidylserine, phytosphingosine) on exposed apoptotic cells. Such binding leads to increased phagocytosis/effercytosis of apoptotic cells by microglia.

CD300LB knockout (KO) mice are protected in a variety of inflammatory models: CD300LB KO mice recover faster from kidney ischemia reperfusion injury (Yamanishi et al., 2010, J. Exp. Med., 207:1501-1511), are protected from joint swelling in zymosan-induced arthritis (Takahashi et al., 2019, Sci. Signal, 12:eaar5514), and have a reduced mortality in models of sepsis (Voss, et al., 2016, J. Immuni., 44:1365-1378; Yamanishi et al., 2012, J. Immunol., 189:1773-1779). The CD300LB receptor can also dimerize with other members of the CD300 family, potentially broadening the signaling pathways induced by ligand-receptor interactions (Martinez-Barriocanal et al., 2010, J. Biol. Chem., 285:41781-41794).

There is a need for novel therapeutic anti-CD300LB antibodies facilitate clustering of CD300LB receptors and activate CD300LB signaling, in part without blocking ligand binding. Additionally, there is a need for novel anti-CD300LB antibodies that are effective at treating or preventing neurodegenerative disorders (e.g., Alzheimer's disease). The present disclosure meets this need by providing anti-CD300LB antibodies that agonize CD300LB activity, such as by increasing CD300LB signaling.

All references cited herein, including patent applications and publications, are hereby incorporated by reference in their entirety.

### SUMMARY OF THE PRESENT DISCLOSURE

The present disclosure is generally directed to anti-CD300LB antibodies and methods of using such antibodies. The methods provided herein find use in increasing phagocytosis/efferocytosis of apoptotic cells by microglia, and in preventing or treating neurodegenerative disorders, such as Alzheimer's disease, in an individual. In some aspects, the present disclosure provides a method for treating an neurodegenerative disorder (e.g., Alzheimer's disease) in an individual, the method comprising administering to the individual in need thereof a therapeutically effective amount of an anti-CD300LB antibody. This disclosure includes many embodiments, including, but not limited to, the following embodiments.

Embodiment 1 is an isolated antibody that specifically binds to CD300LB, wherein the antibody comprises a heavy chain variable region (VH) and a light chain variable region (VL), wherein the heavy chain variable region (VH) comprises:
a. an HVR-H1 comprising an amino acid sequence chosen from any one of SEQ ID NOs:36-46;
b. an HVR-H2 comprising an amino acid sequence chosen from any one of SEQ ID NOs:47-64; and
c. an HVR-H3 comprising an amino acid sequence chosen from any one of SEQ ID NOs:65-79.

Embodiment 2 is the antibody of embodiment 1, wherein the light chain variable region (VL) comprises:
a. an HVR-L1 comprising an amino acid sequence chosen from any one of SEQ ID NOs:80-96;
b. an HVR-L2 comprising an amino acid sequence chosen from any one of SEQ ID NOs:97-111; and
c. an HVR-L3 comprising an amino acid sequence chosen from any one of SEQ ID NOs:112-126.

Embodiment 3 is the antibody of embodiment 1 or 2, wherein the antibody comprises a VH that is at least 90%, at least 95%, at least 97%, or at least 99% identical to an amino acid sequence chosen from any one of SEQ ID NOs:1, 3, 5, 7, 9, 11, 13, 15, 17, 18, 20, 22, 24, 26, 28, 30, 32, and 34.

Embodiment 4 is the antibody of any one of embodiments 1-3, wherein the antibody comprises a VL that is at least 90%, at least 95%, at least 97%, or at least 99% identical to an amino acid sequence chosen from any one of SEQ ID NOs:2, 4, 6, 8, 10, 12, 14, 16, 19, 21, 23, 25, 27, 29, 31, 33, and 35.

Embodiment 5 is the antibody of any one of embodiments 1-4, wherein the VH comprises 1, 2, 3, 4, 5, 6, 7, 8, 9, or 10 amino acid substitutions, insertions, and/or deletions compared to an amino acid sequence chosen from any one of SEQ ID NOs:1, 3, 5, 7, 9, 11, 13, 15, 17, 18, 20, 22, 24, 26, 28, 30, 32, and 34.

Embodiment 6 is the antibody of any one of embodiments 1-5, wherein the VL comprises 1, 2, 3, 4, 5, 6, 7, 8, 9, or 10 amino acid substitutions, insertions, and/or deletions compared to an amino acid sequence chosen from any one of SEQ ID NOs:2, 4, 6, 8, 10, 12, 14, 16, 19, 21, 23, 25, 27, 29, 31, 33, and 35.

Embodiment 7 is the antibody of any one of embodiments 1-6, wherein the antibody comprises a VH comprising an amino acid sequence chosen from any one of SEQ ID NOs:1, 3, 5, 7, 9, 11, 13, 15, 17, 18, 20, 22, 24, 26, 28, 30, 32, and 34.

Embodiment 8 is the antibody of any one of embodiments 1-7, wherein the antibody comprises a VL comprising an amino acid sequence chosen from any one of SEQ ID NOs:2, 4, 6, 8, 10, 12, 14, 16, 19, 21, 23, 25, 27, 29, 31, 33, and 35.

Embodiment 9 is an isolated antibody that specifically binds to human CD300LB, wherein the antibody comprises a VH comprising HVR-H1, HVR-H2, and HVR-H3 and a VL comprising HVR-L1, HVR-L2, and HVR-L3 of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

Embodiment 10 is the isolated antibody of embodiment 9, wherein the antibody comprises a VH and/or a VL at least 90%, at least 95%, at least 97%, or at least 99% identical to those of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

Embodiment 11 is the isolated antibody of embodiment 9 or embodiment 10, wherein the antibody comprises the VH and/or the VL of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

Embodiment 12 is an isolated antibody that specifically binds to human CD300LB, wherein the antibody comprises:
a. a VH comprising an HVR-H1 comprising the amino acid sequence of SEQ ID NO:36, an HVR-H2 comprising the amino acid sequence of SEQ ID NO:47, an HVR-H3 comprising the amino acid sequence of SEQ ID NO:65; and a VL comprising an HVR-L1 comprising the amino acid sequence of SEQ ID NO:80, an HVR-L2 comprising the amino acid sequence of SEQ ID NO:97, and an HVR-L3 comprising the amino acid sequence of SEQ ID NO: 112;
b. a VH comprising an HVR-H1 comprising the amino acid sequence of SEQ ID NO:37, an HVR-H2 comprising the amino acid sequence of SEQ ID NO:48; an HVR-H3 comprising the amino acid sequence of SEQ ID NO:66, an HVR-L1 comprising the amino acid sequence of SEQ ID NO:81; and a VL comprising an HVR-L2 comprising the amino acid sequence of SEQ ID NO:98, and an HVR-L3 comprising the amino acid sequence of SEQ ID NO: 113;
c. a VH comprising an HVR-H1 comprising the amino acid sequence of SEQ ID NO:38, an HVR-H2 comprising the amino acid sequence of SEQ ID NO:49, an HVR-H3 comprising the amino acid sequence of SEQ ID NO:67; and a VL comprising an HVR-L1 comprising the amino acid sequence of SEQ ID NO:82, an HVR-L2 comprising the amino acid sequence of SEQ ID NO:99, and an HVR-L3 comprising the amino acid sequence of SEQ ID NO: 114;
d. a VH comprising an HVR-H1 comprising the amino acid sequence of SEQ ID NO:36, an HVR-H2 comprising the amino acid sequence of SEQ ID NO:50, an HVR-H3 comprising the amino acid sequence of SEQ ID NO:68; and a VL comprising an HVR-L1 comprising the amino acid sequence of SEQ ID NO:83, an HVR-L2 comprising the amino acid sequence of SEQ ID NO: 100, and an HVR-L3 comprising the amino acid sequence of SEQ ID NO: 112;
e. a VH comprising an HVR-H1 comprising the amino acid sequence of SEQ ID NO:39, an HVR-H2 comprising the amino acid sequence of SEQ ID NO:51, an HVR-H3 comprising the amino acid sequence of SEQ ID NO:69; and a VL comprising an HVR-L1 comprising the amino acid sequence of SEQ ID NO:84, an HVR-L2 comprising the amino acid sequence of SEQ ID NO:101, and an HVR-L3 comprising the amino acid sequence of SEQ ID NO: 115;
f. a VH comprising an HVR-H1 comprising the amino acid sequence of SEQ ID NO: 40, an HVR-H2 comprising the amino acid sequence of SEQ ID NO:52, an HVR-H3 comprising the amino acid sequence of SEQ ID NO:70; and a VL comprising an HVR-L1 comprising the amino acid sequence of SEQ ID NO:85, an HVR-L2 comprising the amino acid sequence of SEQ ID NO: 102; and an HVR-L3 comprising the amino acid sequence of SEQ ID NO: 116;
g. a VH comprising an HVR-H1 comprising the amino acid sequence of SEQ ID NO:41, an HVR-H2 comprising the amino acid sequence of SEQ ID NO:53, an HVR-H3 comprising the amino acid sequence of SEQ ID NO:71; and a VL comprising an HVR-L1 comprising the amino acid sequence of SEQ ID NO:86, an HVR-L2 comprising the amino acid sequence of SEQ ID NO: 103, and an HVR-L3 comprising the amino acid sequence of SEQ ID NO: 117;
h. a VH comprising an HVR-H1 comprising the amino acid sequence of SEQ ID NO: 42, an HVR-H2 comprising the amino acid sequence of SEQ ID NO:54, an HVR-H3 comprising the amino acid sequence of SEQ ID NO:72; and a VL comprising an HVR-L1 comprising the amino acid sequence of SEQ ID NO:87, an HVR-L2 comprising the amino acid sequence of SEQ ID NO: 104, and an HVR-L3 comprising the amino acid sequence of SEQ ID NO: 118;
i. a VH comprising an HVR-H1 comprising the amino acid sequence of SEQ ID NO: 40, an HVR-H2 comprising the amino acid sequence of SEQ ID NO:55, an HVR-H3 comprising the amino acid sequence of SEQ ID NO:70; and a VL comprising an HVR-L1 comprising the amino acid sequence of SEQ ID NO:85, an HVR-L2 comprising the amino acid sequence of SEQ ID NO: 102, and an HVR-L3 comprising the amino acid sequence of SEQ ID NO:116;
j. a VH comprising an HVR-H1 comprising the amino acid sequence of SEQ ID NO:43, an HVR-H2 comprising the amino acid sequence of SEQ ID NO:56, an HVR-H3 comprising the amino acid sequence of SEQ ID NO:73; and a VL comprising an HVR-L1 comprising the amino acid sequence of SEQ ID NO:88, an HVR-L2 comprising the amino acid sequence of SEQ ID NO: 105, and an HVR-L3 comprising the amino acid sequence of SEQ ID NO: 119;
k. a VH comprising an HVR-H1 comprising the amino acid sequence of SEQ ID NO: 44, an HVR-H2 comprising the amino acid sequence of SEQ ID NO:57, an HVR-H3 comprising the amino acid sequence of SEQ ID NO:74; and a VL comprising an HVR-L1 comprising the amino acid sequence of SEQ ID NO:89, an HVR-L2 comprising the amino acid sequence of SEQ ID NO: 106, and an HVR-L3 comprising the amino acid sequence of SEQ ID NO: 120;
l. a VH comprising an HVR-H1 comprising the amino acid sequence of SEQ ID NO: 44, an HVR-H2 comprising the amino acid sequence of SEQ ID NO:58, an HVR-H3 comprising the amino acid sequence of SEQ ID NO:75; and a VL comprising an HVR-L1 comprising the amino acid sequence of SEQ ID NO:90, an HVR-L2 comprising the amino acid sequence of SEQ ID NO: 107, and an HVR-L3 comprising the amino acid sequence of SEQ ID NO:121;
m. a VH comprising an HVR-H1 comprising the amino acid sequence of SEQ ID NO: 44, an HVR-H2 comprising the amino acid sequence of SEQ ID NO:59, an HVR-H3 comprising the amino acid sequence of SEQ ID NO:75; and a VL comprising an HVR-L1 comprising the amino acid sequence of SEQ ID NO:91, an HVR-L2 comprising the amino acid sequence of SEQ ID NO:108, and an HVR-L3 comprising the amino acid sequence of SEQ ID NO: 122;
n. a VH comprising an HVR-H1 comprising the amino acid sequence of SEQ ID NO: 45, an HVR-H2 comprising the amino acid sequence of SEQ ID NO:60, an HVR-H3 comprising the amino acid sequence of SEQ ID NO:76; and a VL comprising an HVR-L1 comprising the amino acid sequence of SEQ ID NO:92, an HVR-L2 comprising the amino acid sequence of SEQ ID NO: 109, and an HVR-L3 comprising the amino acid sequence of SEQ ID NO: 123;
o. a VH comprising an HVR-H1 comprising the amino acid sequence of SEQ ID NO: 44, an HVR-H2 comprising the amino acid sequence of SEQ ID NO:61, an HVR-H3 comprising the amino acid sequence of SEQ ID NO:77; and a VL comprising an HVR-L1 comprising the amino acid sequence of SEQ ID NO:93, an HVR-L2 comprising the amino acid sequence of SEQ ID NO: 110, and an HVR-L3 comprising the amino acid sequence of SEQ ID NO: 121;
p. a VH comprising an HVR-H1 comprising the amino acid sequence of SEQ ID NO: 44, an HVR-H2 comprising the amino acid sequence of SEQ ID NO:62, an HVR-H3 comprising the amino acid sequence of SEQ ID NO:77; and a VL comprising an HVR-L1 comprising the amino acid sequence of SEQ ID NO:94, an HVR-L2 comprising the amino acid sequence of SEQ ID NO:110, and an HVR-L3 comprising the amino acid sequence of SEQ ID NO: 124;
q. a VH comprising an HVR-H1 comprising the amino acid sequence of SEQ ID NO:44, HVR-H2 comprising the amino acid sequence of SEQ ID NO:63, an HVR-H3 comprising the amino acid sequence of SEQ ID NO:78; and a VL comprising an HVR-L1 comprising the amino acid sequence of SEQ ID NO:95, an HVR-L2 comprising the amino acid sequence of SEQ ID NO: 107, and an HVR-L3 comprising the amino acid sequence of SEQ ID NO: 125; or
r. a VH comprising an HVR-H1 comprising the amino acid sequence of SEQ ID NO:46. an HVR-H2 comprising the amino acid sequence of SEQ ID NO:64, an HVR-H3 comprising the amino acid sequence of SEQ ID NO:79; and a VL comprising an HVR-L1 comprising the amino acid sequence of SEQ ID NO:96, an HVR-L2 comprising the amino acid sequence of SEQ ID NO:111, and an HVR-L3 comprising the amino acid sequence of SEQ ID NO: 126.

Embodiment 13 is the antibody of embodiment 12, wherein the antibody comprises:
a. the HVRs of embodiment 12.a. and further comprises a VH that is at least 90%, at least 95%, at least 97%, or at least 99% identical to the amino acid sequence of SEQ ID NO: 1;
b. the HVRs of embodiment 12.b. and further comprises a VH that is at least 90%, at least 95%, at least 97%, or at least 99% identical to the amino acid sequence of SEQ ID NO:3;
c. the HVRs of embodiment 12.c. and further comprises a VH that is at least 90%, at least 95%, at least 97%, or at least 99% identical to the amino acid sequence of SEQ ID NO:5;
d. the HVRs of embodiment 12.d. and further comprises a VH that is at least 90%, at least 95%, at least 97%, or at least 99% identical to the amino acid sequence of SEQ ID NO:7;
e. the HVRs of embodiment 12.e. and further comprises a VH that is at least 90%, at least 95%, at least 97%, or at least 99% identical to the amino acid sequence of SEQ ID NO:9;
f. the HVRs of embodiment 12.f. and further comprises a VH that is at least 90%, at least 95%, at least 97%, or at least 99% identical to the amino acid sequence of SEQ ID NO:11;
g. the HVRs of embodiment 12.g. and further comprises a VH that is at least 90%, at least 95%, at least 97%, or at least 99% identical to the amino acid sequence of SEQ ID NO:13;
h. the HVRs of embodiment 12.h. and further comprises a VH that is at least 90%, at least 95%, at least 97%, or at least 99% identical to the amino acid sequence of SEQ ID NO:15;
i. the HVRs of embodiment 12.i. and further comprises a VH that is at least 90%, at least 95%, at least 97%, or at least 99% identical to the amino acid sequence of SEQ ID NO:17;
j. the HVRs of embodiment 12.j. and further comprises a VH that is at least 90%, at least 95%, at least 97%, or at least 99% identical to the amino acid sequence of SEQ ID NO:18;
k. the HVRs of embodiment 12k. and further comprises a VH that is at least 90%, at least 95%, at least 97%, or at least 99% identical to the amino acid sequence of SEQ ID NO:20;
l. the HVRs of embodiment 12.1. and further comprises a VH that is at least 90%, at least 95%, at least 97%, or at least 99% identical to the amino acid sequence of SEQ ID NO:22;
m. the HVRs of embodiment 12.m. and further comprises a VH that is at least 90%, at least 95%, at least 97%, or at least 99% identical to the amino acid sequence of SEQ ID NO:24;
n. the HVRs of embodiment 12.n. and further comprises a VH that is at least 90%, at least 95%, at least 97%, or at least 99% identical to the amino acid sequence of SEQ ID NO:26;
o. the HVRs of embodiment 12.o. and further comprises a VH that is at least 90%, at least 95%, at least 97%, or at least 99% identical to the amino acid sequence of SEQ ID NO:28;
p. the HVRs of embodiment 12.p. and further comprises a VH that is at least 90%, at least 95%, at least 97%, or at least 99% identical to the amino acid sequence of SEQ ID NO:30;
q. the HVRs of embodiment 12.q. and further comprises a VH that is at least 90%, at least 95%, at least 97%, or at least 99% identical to the amino acid sequence of SEQ ID NO:32; or
r. the HVRs of embodiment 12.r. and further comprises a VH that is at least 90%, at least 95%, at least 97%, or at least 99% identical to the amino acid sequence of SEQ ID NO:34.

Embodiment 14 is the antibody of embodiment 12 or 13, wherein the antibody comprises: a. the HVRs of embodiment 12.a. and further comprises a VL that is at least 90%, at least 95%, at least 97%, or at least 99% identical to the amino acid sequence of SEQ ID NO: 2;
b. the HVRs of embodiment 12.b. and further comprises a VL that is at least 90%, at least 95%, at least 97%, or at least 99% identical to the amino acid sequence of SEQ ID NO: 4;
c. the HVRs of embodiment 12.c. and further comprises a VL that is at least 90%, at least 95%, at least 97%, or at least 99% identical to the amino acid sequence of SEQ ID NO: 6;
d. the HVRs of embodiment 12.d. and further comprises a VL that is at least 90%, at least 95%, at least 97%, or at least 99% identical to the amino acid sequence of SEQ ID NO: 8;
e. the HVRs of embodiment 12.e. and further comprises a VL that is at least 90%, at least 95%, at least 97%, or at least 99% identical to the amino acid sequence of SEQ ID NO: 10;
f. the HVRs of embodiment 12.f. and further comprises a VL that is at least 90%, at least 95%, at least 97%, or at least 99% identical to the amino acid sequence of SEQ ID NO: 12;
g. the HVRs of embodiment 12.g. and further comprises a VL that is at least 90%, at least 95%, at least 97%, or at least 99% identical to the amino acid sequence of SEQ ID NO: 14;
h. the HVRs of embodiment 12.h. and further comprises a VL that is at least 90%, at least 95%, at least 97%, or at least 99% identical to the amino acid sequence of SEQ ID NO: 16;
i. the HVRs of embodiment 12.i. and further comprises a VL that is at least 90%, at least 95%, at least 97%, or at least 99% identical to the amino acid sequence of SEQ ID NO: 12;
j. the HVRs of embodiment 12.j. and further comprises a VL that is at least 90%, at least 95%, at least 97%, or at least 99% identical to the amino acid sequence of SEQ ID NO: 19;
k. the HVRs of embodiment 12k. and further comprises a VL that is at least 90%, at least 95%, at least 97%, or at least 99% identical to the amino acid sequence of SEQ ID NO: 21;
l. the HVRs of embodiment 12.1. and further comprises a VL that is at least 90%, at least 95%, at least 97%, or at least 99% identical to the amino acid sequence of SEQ ID NO: 23;
m. the HVRs of embodiment 12.m. and further comprises a VL that is at least 90%, at least 95%, at least 97%, or at least 99% identical to the amino acid sequence of SEQ ID NO: 25;
n. the HVRs of embodiment 12.n. and further comprises a VL that is at least 90%, at least 95%, at least 97%, or at least 99% identical to the amino acid sequence of SEQ ID NO: 27;
o. the HVRs of embodiment 12.o. and further comprises a VL that is at least 90%, at least 95%, at least 97%, or at least 99% identical to the amino acid sequence of SEQ ID NO: 29;
p. the HVRs of embodiment 12.p. and further comprises a VL that is at least 90%, at least 95%, at least 97%, or at least 99% identical to the amino acid sequence of SEQ ID NO: 31;
q. the HVRs of embodiment 12.q. and further comprises a VL that is at least 90%, at least 95%, at least 97%, or at least 99% identical to the amino acid sequence of SEQ ID NO: 33; or
r. the HVRs of embodiment 12.r. and further comprises a VL that is at least 90%, at least 95%, at least 97%, or at least 99% identical to the amino acid sequence of SEQ ID NO: 35.

Embodiment 15 is the antibody of any one of embodiments 12-14, wherein the antibody comprises:
a. the HVRs of embodiment 12.a. and further comprises a VH comprising the amino acid sequence of SEQ ID NO:1;
b. the HVRs of embodiment 12.b. and further comprises a VH comprising the amino acid sequence of SEQ ID NO:3;
c. the HVRs of embodiment 12.c. and further comprises a VH comprising the amino acid sequence of SEQ ID NO:5;
d. the HVRs of embodiment 12.d. and further comprises a VH comprising the amino acid sequence of SEQ ID NO:7;
e. the HVRs of embodiment 12.e. and further comprises a VH comprising the amino acid sequence of SEQ ID NO:9;
f. the HVRs of embodiment 12.f. and further comprises a VH comprising the amino acid sequence of SEQ ID NO:11;
g. the HVRs of embodiment 12.g. and further comprises a VH comprising the amino acid sequence of SEQ ID NO:13;
h. the HVRs of embodiment 12.h. and further comprises a VH comprising the amino acid sequence of SEQ ID NO:15;
i. the HVRs of embodiment 12.i. and further comprises a VH comprising the amino acid sequence of SEQ ID NO:17;
j. the HVRs of embodiment 12.j. and further comprises a VH comprising the amino acid sequence of SEQ ID NO:18;
k. the HVRs of embodiment 12k. and further comprises a VH comprising the amino acid sequence of SEQ ID NO:20;
l. the HVRs of embodiment 12.1. and further comprises a VH comprising the amino acid sequence of SEQ ID NO:22;
m. the HVRs of embodiment 12.m. and further comprises a VH comprising the amino acid sequence of SEQ ID NO:24;
n. the HVRs of embodiment 12.n. and further comprises a VH comprising the amino acid sequence of SEQ ID NO:26;
o. the HVRs of embodiment 12.o. and further comprises a VH comprising the amino acid sequence of SEQ ID NO:28;
p. the HVRs of embodiment 12.p. and further comprises a VH comprising the amino acid sequence of SEQ ID NO:30;
q. the HVRs of embodiment 12.q. and further comprises a VH comprising the amino acid sequence of SEQ ID NO:32; or
r. the HVRs of embodiment 12.r. and further comprises a VH comprising the amino acid sequence of SEQ ID NO:34.

Embodiment 16 is the antibody of any one of embodiments 12-15, wherein the antibody comprises:
a. the HVRs of embodiment 12.a. and further comprises a VL comprising the amino acid sequence of SEQ ID NO: 2;
b. the HVRs of embodiment 12.b. and further comprises a VL comprising the amino acid sequence of SEQ ID NO: 4;
c. the HVRs of embodiment 12.c. and further comprises a VL comprising the amino acid sequence of SEQ ID NO: 6;
d. the HVRs of embodiment 12.d. and further comprises a VL comprising the amino acid sequence of SEQ ID NO: 8;
e. the HVRs of embodiment 12.e. and further comprises a VL comprising the amino acid sequence of SEQ ID NO: 10;
f. the HVRs of embodiment 12.f. and further comprises a VL comprising the amino acid sequence of SEQ ID NO: 12;
g. the HVRs of embodiment 12.g. and further comprises a VL comprising the amino acid sequence of SEQ ID NO: 14;
h. the HVRs of embodiment 12.h. and further comprises a VL comprising the amino acid sequence of SEQ ID NO: 16;
i. the HVRs of embodiment 12.i. and further comprises a VL comprising the amino acid sequence of SEQ ID NO: 12;
j. the HVRs of embodiment 12.j. and further comprises a VL comprising the amino acid sequence of SEQ ID NO: 19;
k. the HVRs of embodiment 12k. and further comprises a VL comprising the amino acid sequence of SEQ ID NO: 21;
l. the HVRs of embodiment 12.1. and further comprises a VL comprising the amino acid sequence of SEQ ID NO: 23;
m. the HVRs of embodiment 12.m. and further comprises a VL comprising the amino acid sequence of SEQ ID NO: 25;
n. the HVRs of embodiment 12.n. and further comprises a VL comprising the amino acid sequence of SEQ ID NO: 27;
o. the HVRs of embodiment 12.o. and further comprises a VL comprising the amino acid sequence of SEQ ID NO: 29;
p. the HVRs of embodiment 12.p. and further comprises a VL comprising the amino acid sequence of SEQ ID NO: 31;
q. the HVRs of embodiment 12.q. and further comprises a VL comprising the amino acid sequence of SEQ ID NO: 33; or
r. the HVRs of embodiment 12.r. and further comprises a VL comprising the amino acid sequence of SEQ ID NO: 35.

Embodiment 17 is the antibody of any one of embodiments 9-12, wherein the antibody comprises
a. a VH comprising the amino acid sequence of SEQ ID NO: 1 and a VL comprising the amino acid sequence of SEQ ID NO: 2 (as shown in Table 13);
b. a VH comprising the amino acid sequence of SEQ ID NO: 3 and a VL comprising the amino acid sequence of SEQ ID NO: 4 (as shown in Table 13);
c. a VH comprising the amino acid sequence of SEQ ID NO: 5 and a VL comprising the amino acid sequence of SEQ ID NO: 6 (as shown in Table 13);
d. a VH comprising the amino acid sequence of SEQ ID NO: 7 and a VL comprising the amino acid sequence of SEQ ID NO: 8 (as shown in Table 13);
e. a VH comprising the amino acid sequence of SEQ ID NO: 9 and a VL comprising the amino acid sequence of SEQ ID NO: 10 (as shown in Table 13);
f. a VH comprising the amino acid sequence of SEQ ID NO: 11 and a VL comprising the amino acid sequence of SEQ ID NO: 12 (as shown in Table 13);
g. a VH comprising the amino acid sequence of SEQ ID NO: 13 and a VL comprising the amino acid sequence of SEQ ID NO: 14 (as shown in Table 13);
h. a VH comprising the amino acid sequence of SEQ ID NO: 15 and a VL comprising the amino acid sequence of SEQ ID NO: 16 (as shown in Table 13);
i. a VH comprising the amino acid sequence of SEQ ID NO: 17 and a VL comprising the amino acid sequence of SEQ ID NO: 12 (as shown in Table 13);
j. a VH comprising the amino acid sequence of SEQ ID NO: 18 and a VL comprising the amino acid sequence of SEQ ID NO: 19 (as shown in Table 13);
k. a VH comprising the amino acid sequence of SEQ ID NO: 20 and a VL comprising the amino acid sequence of SEQ ID NO: 21 (as shown in Table 13);
l. a VH comprising the amino acid sequence of SEQ ID NO: 22 and a VL comprising the amino acid sequence of SEQ ID NO: 23 (as shown in Table 13);
m. a VH comprising the amino acid sequence of SEQ ID NO: 24 and a VL comprising the amino acid sequence of SEQ ID NO: 25 (as shown in Table 13);
n. a VH comprising the amino acid sequence of SEQ ID NO: 26 and a VL comprising the amino acid sequence of SEQ ID NO: 27 (as shown in Table 13);
o. a VH comprising the amino acid sequence of SEQ ID NO: 28 and a VL comprising the amino acid sequence of SEQ ID NO: 29 (as shown in Table 13);
p. a VH comprising the amino acid sequence of SEQ ID NO: 30 and a VL comprising the amino acid sequence of SEQ ID NO: 31 (as shown in Table 13);
q. a VH comprising the amino acid sequence of SEQ ID NO: 32 and a VL comprising the amino acid sequence of SEQ ID NO: 33 (as shown in Table 13); or
r. a VH comprising the amino acid sequence of SEQ ID NO: 34 and a VL comprising the amino acid sequence of SEQ ID NO: 35 (as shown in Table 13).

Embodiment 18 is an isolated antibody that specifically binds to human CD300LB, wherein the antibody comprises:
a. a VH and VL, wherein the VH comprises the HVR-H1, HVR-H2, and HVR-H3 of the antibody CD-01 (as shown in Table 14) and the VL comprises the HVR-L1, HVR-L2, and HVR-L3 of the antibody CD-01 (as shown in Table 15);
b. VH and VL, wherein the VH comprises the HVR-H1, HVR-H2, and HVR-H3 of the antibody CD-02 (as shown in Table 14) and the VL comprises the HVR-L1, HVR-L2, and HVR-L3 of the antibody CD-01 (as shown in Table 15);
c. VH and VL, wherein the VH comprises the HVR-H1, HVR-H2, and HVR-H3 of the antibody CD-03 (as shown in Table 14) and the VL comprises the HVR-L1, HVR-L2, and HVR-L3 of the antibody CD-01 (as shown in Table 15);
d. VH and VL, wherein the VH comprises the HVR-H1, HVR-H2, and HVR-H3 of the antibody CD-04 (as shown in Table 14) and the VL comprises the HVR-L1, HVR-L2, and HVR-L3 of the antibody CD-01 (as shown in Table 15);
e. VH and VL, wherein the VH comprises the HVR-H1, HVR-H2, and HVR-H3 of the antibody CD-05 (as shown in Table 14) and the VL comprises the HVR-L1, HVR-L2, and HVR-L3 of the antibody CD-01 (as shown in Table 15);
f. VH and VL, wherein the VH comprises the HVR-H1, HVR-H2, and HVR-H3 of the antibody CD-06 (as shown in Table 14) and the VL comprises the HVR-L1, HVR-L2, and HVR-L3 of the antibody CD-01 (as shown in Table 15);
g. VH and VL, wherein the VH comprises the HVR-H1, HVR-H2, and HVR-H3 of the antibody CD-07 (as shown in Table 14) and the VL comprises the HVR-L1, HVR-L2, and HVR-L3 of the antibody CD-01 (as shown in Table 15);
h. VH and VL, wherein the VH comprises the HVR-H1, HVR-H2, and HVR-H3 of the antibody CD-08 (as shown in Table 14) and the VL comprises the HVR-L1, HVR-L2, and HVR-L3 of the antibody CD-01 (as shown in Table 15);
i. VH and VL, wherein the VH comprises the HVR-H1, HVR-H2, and HVR-H3 of the antibody CD-09 (as shown in Table 14) and the VL comprises the HVR-L1, HVR-L2, and HVR-L3 of the antibody CD-01 (as shown in Table 15);
j. VH and VL, wherein the VH comprises the HVR-H1, HVR-H2, and HVR-H3 of the antibody CD-10 (as shown in Table 14) and the VL comprises the HVR-L1, HVR-L2, and HVR-L3 of the antibody CD-01 (as shown in Table 15);
k. VH and VL, wherein the VH comprises the HVR-H1, HVR-H2, and HVR-H3 of the antibody CD-11 (as shown in Table 14) and the VL comprises the HVR-L1, HVR-L2, and HVR-L3 of the antibody CD-01 (as shown in Table 15);
l. VH and VL, wherein the VH comprises the HVR-H1, HVR-H2, and HVR-H3 of the antibody CD-12 (as shown in Table 14) and the VL comprises the HVR-L1, HVR-L2, and HVR-L3 of the antibody CD-01 (as shown in Table 15);
m. VH and VL, wherein the VH comprises the HVR-H1, HVR-H2, and HVR-H3 of the antibody CD-13 (as shown in Table 14) and the VL comprises the HVR-L1, HVR-L2, and HVR-L3 of the antibody CD-01 (as shown in Table 15);
n. VH and VL, wherein the VH comprises the HVR-H1, HVR-H2, and HVR-H3 of the antibody CD-14 (as shown in Table 14) and the VL comprises the HVR-L1, HVR-L2, and HVR-L3 of the antibody CD-01 (as shown in Table 15);
o. VH and VL, wherein the VH comprises the HVR-H1, HVR-H2, and HVR-H3 of the antibody CD-15 (as shown in Table 14) and the VL comprises the HVR-L1, HVR-L2, and HVR-L3 of the antibody CD-01 (as shown in Table 15);
p. VH and VL, wherein the VH comprises the HVR-H1, HVR-H2, and HVR-H3 of the antibody CD-16 (as shown in Table 14) and the VL comprises the HVR-L1, HVR-L2, and HVR-L3 of the antibody CD-01 (as shown in Table 15);
q. VH and VL, wherein the VH comprises the HVR-H1, HVR-H2, and HVR-H3 of the antibody CD-17 (as shown in Table 14) and the VL comprises the HVR-L1, HVR-L2, and HVR-L3 of the antibody CD-01 (as shown in Table 15); or
r. VH and VL, wherein the VH comprises the HVR-H1, HVR-H2, and HVR-H3 of the antibody CD-18 (as shown in Table 14) and the VL comprises the HVR-L1, HVR-L2, and HVR-L3 of the antibody CD-01 (as shown in Table 15).

Embodiment 19 is the antibody of any one of embodiments 1-18, wherein the antibody has one or more of the following properties:
a. the antibody increases the activity of human CD300LB and/or mouse CD300LB;
b. the antibody specifically binds to both human CD300LB and cynomolgus monkey CD300LB;
c. the antibody does not bind to murine CD300LB;
d. the antibody binds to to human CD300LB on the surface of cells overexpressing human CD300LB with a KD of less than 1 μM, less than 100 nM, less than 10 nM, less than 1 nM;
e. the antibody has a higher affinity for human CD300LB than to human CD300C, human CD300D, human CD300E, and/or human CD300F;
f. the antibody does not bind to human CD300C, human CD300D, human CD300E, human CD300F, mouse CD300D, and/or mouse CD300F;
g. the antibody activates plate-bound CD300LB, for example expressed in a mouse BaF/3 cell line;
h. the antibody binds to human monocytes and human macrophages;
i. the antibody increases Syk phosphorylation in primary human macrophages; and
j. the antibody induces DAP12 tyrosine phosphorylation in wild-type mouse bone marrow derived macrophages.

Embodiment 20 is the antibody of any one of embodiments 1-19, wherein the antibody is a monoclonal antibody.

Embodiment 21 is the antibody of any one of embodiments 1-20, wherein the antibody is a humanized antibody.

Embodiment 22 is the antibody of any one of embodiments 1-21, wherein the antibody is an antigen binding fragment, such as an Fab, Fab′, Fab′-SH, F(ab′)₂, Fv, or scFv fragment.

Embodiment 23 is the antibody of any one of embodiments 1-22, wherein the antibody is a bispecific or multispecific antibody.

Embodiment 24 is the antibody of any one of embodiments 1-23, wherein the antibody is of the IgG class, the IgM class, or the IgA class.

Embodiment 25 is the antibody of embodiment 24, wherein the antibody is of the IgG class and is of a human IgG1, IgG2, IgG3, or IgG4 isotype or of a mouse IgG1 or IgG2 isotype.

Embodiment 26 is the antibody of any one of embodiments 1-25, wherein the antibody binds to an inhibitory Fc receptor.

Embodiment 27 is the antibody of embodiment 26, wherein the inhibitory Fc receptor is inhibitory Fc-gamma receptor IIB (FcgRIIB).

Embodiment 28 is the antibody of embodiment 27, wherein the antibody decreases cellular levels of FcgRIIB.

Embodiment 29 is the antibody of any one of embodiments 1-28, wherein the anti-CD300LB antibody has a human or mouse IgG1 isotype and comprises one or more amino acid substitutions in the Fc region at an amino acid residue selected from the group consisting of N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, N325S, T394D, A330S, E430G, E430S, E430F, E430T, E345K, E345Q, E345R, E345Y, S440Y, S440W, and any combination thereof, wherein the numbering of the residues is according to EU numbering.

Embodiment 30 is the antibody of any one of embodiments 1-28, wherein the antibody has a human or mouse IgG2 isotype and comprises one or more amino acid substitutions in the Fc region at an amino acid residue selected from the group consisting of A330S, C127S, C214S, C219S, C220S, E345K, E345Q, E345R, E345Y, E430F, E430G, E430S, E430T, G237A, H268Q, L328F, M252Y, P331S, S254T, S267E, S440W, S440Y, T256E, V234A, V309L, and any combination thereof, wherein the numbering of the residues is according to EU numbering.

Embodiment 31 is the antibody of any one of embodiments 1-28, wherein the anti-CD300LB antibody has a human or mouse IgG4 isotype and comprises one or more amino acid substitutions in the Fc region at an amino acid residue selected from the group consisting of C127S, E318A, E345R, E430G, F234A, G237A, K322A, L235A, L235E, L236E, L243A, L328F, M252Y, P331S, S228P, S229P, S254T, S267E, S440Y, T256E, and any combination thereof, wherein the numbering of the residues is according to EU numbering.

Embodiment 32 is the antibody of any one of embodiments 1-31, wherein the antibody comprises one or more amino acid substitutions in the Fc region at a residue position selected from the group consisting of A330L, A330S, C127S, E345R, E430G, K322A, L234A, L234F, L235A, L235E, L243A, L328F, P331S, S267E, S440Y, and any combination thereof, wherein the numbering of the amino acid residues is according to EU or Kabat numbering.

Embodiment 33 is a pharmaceutical composition comprising the anti-CD300LB antibody of any one of embodiments 1-32 and a pharmaceutically acceptable carrier.

Embodiment 34 is an isolated nucleic acid comprising a nucleic acid sequence encoding the anti-CD300LB antibody of any one of embodiments 1-32.

Embodiment 35 is an isolated vector comprising the nucleic acid of embodiment 34.

Embodiment 36 is an isolated host cell comprising the nucleic acid of embodiment 34 or the vector of embodiment 35.

Embodiment 37 is a method of producing an antibody that binds to human CD300LB, comprising culturing the cell of embodiment 36 so that the antibody is produced.

Embodiment 38 is the method of embodiment 37, further comprising recovering the antibody produced by the cell.

Embodiment 39 is a method of treating a neurodegenerative disease or disorder, the method comprising administering to an individual in need thereof a therapeutically effective amount of an anti-CD300LB antibody of any one of embodiments 1-32, thereby treating the disease or disorder.

Embodiment 40 is the method of embodiment 39, wherein the disease or disorder is Alzheimer's disease.

Embodiment 41 is a method of detecting the presence of CD300LB in a sample or an individual, the method comprising an anti-CD300LB antibody of any one of embodiments 1-32.

Embodiment 42 is the method of embodiment 41, further comprising quantification of antigen-bound anti-CD300LB antibody.

In one aspect, the present disclosure relates to an isolated antibody that binds to a CD300LB protein, wherein the antibody competitively inhibits binding of one or more of the antibodies of any of the aspects herein for binding to CD300LB.

In another aspect, the present disclosure relates to an isolated antibody that binds to a CD300LB protein, wherein the antibody binds essentially the same or an overlapping epitope on CD300LB as an antibody of any of the aspects herein. In another aspect, the present disclosure relates to an isolated antibody that binds to a CD300LB protein, wherein the antibody binds the same epitope on CD300LB as an antibody of any of the aspects herein.

In certain aspects that may be combined with any of the aspects herein, the CD300LB protein is a mammalian protein or a human protein. In certain aspects that may be combined with any of the aspects herein, the CD300LB protein is a wild-type protein. In certain aspects that may be combined with any of the aspects herein, the CD300LB protein is a naturally occurring variant. In certain aspects that may be combined with any of the aspects herein, an anti-CD300LB antibody binds to human CD300LB, and to cynomolgus monkey CD300LB and/or murine CD300LB.

In some aspects that may be combined with any of the aspects herein, an anti-CD300LB antibody of the present disclosure does not inhibit or reduce binding of one or more ligands to CD300LB. In some aspects that may be combined with any of the aspects herein, an anti-CD300LB antibody of the present disclosure does not inhibit or reduce binding of phosphatidylserine to CD300LB. In some aspects that may be combined with any of the aspects herein, an anti-CD300LB antibody of the present disclosure does not inhibit or reduce binding of phytosphingosine to CD300LB. In some aspects that may be combined with any of the aspects herein, an anti-CD300LB antibody of the present disclosure does not inhibit or reduce binding of phosphatidylserine to CD300LB and does not inhibit or reduce binding of phytosphingosine to CD30LB.

In some aspects that may be combined with any of the aspects provided herein, an anti-CD300LB antibody of the present disclosure increases phagocytosis by a phagocytic cell. In some aspects that may be combined with any of the aspects provided herein, an anti-CD300LB antibody of the present disclosure increases clearance of myelin debris by phagocytosis. In some aspects that may be combined with any of the aspects provided herein, an anti-CD300LB antibody of the present disclosure increases efferocytosis by a phagocytic cell. In some aspects that may be combined with any of the aspects provided herein, an anti-CD300LB antibody of the present disclosure increases efferocytosis of apoptotic cells by a phagocytic cell.

In some aspects that may be combined with any of the aspects provided herein, an anti-CD300LB antibody of the present disclosure increases Syk phosphorylation. In some aspects that may be combined with any of the aspects provided herein, an anti-CD300LB antibody of the present disclosure increases Syk phosphorylation in a myeloid cell, including without limitation a monocyte, a macrophage, a dendritic cell, and a microglial cell.

In some aspects that may be combined with any of the aspects provided herein, an anti-CD300LB antibody of the present disclosure binds to human CD300LB. In some aspects that may be combined with any of the aspects provided herein, an anti-CD300LB antibody of the present disclosure binds to human CD300LB and to murine CD300LB. In some aspects that may be combined with any of the aspects provided herein, an anti-CD300LB antibody of the present disclosure binds human CD300LB with an affinity of about 15-329 nM.

In some aspects that may be combined with any of the aspects herein, the anti-CD300LB antibody of the present disclosure is a monoclonal antibody. In some aspects that may be combined with any of the aspects herein, the antibody is a human antibody. In some aspects that may be combined with any of the aspects herein, the antibody is a humanized antibody. In some aspects that may be combined with any of the aspects herein, the antibody is a bispecific antibody. In some aspects that may be combined with any of the aspects herein, the antibody is a multivalent antibody. In some aspects that may be combined with any of the aspects herein, the antibody is a chimeric antibody.

In some aspects that may be combined with any of the aspects herein, the anti-CD300LB antibody of the present disclosure is of the IgG class, the IgM class, or the IgA class. In some aspects, the antibody is of the IgG class and has an IgG1, IgG2, or IgG4 isotype. In certain aspects that may be combined with any of the aspects herein, the antibody is a full-length antibody. In certain aspects that may be combined with any of the aspects herein, the antibody is an antibody fragment. In certain aspects that may be combined with any of the aspects herein, the antibody is an antibody fragment that binds to an epitope on human CD300LB or a mammalian CD300LB protein. In certain aspects that may be combined with any of the aspects herein, the antibody fragment is a Fab, Fab′, Fab′-SH, F(ab′)2, Fv, or scFv fragment.

In another aspect, the present disclosure relates to an isolated nucleic acid comprising a nucleic acid sequence encoding an anti-CD300LB antibody of any of the preceding aspects. In some aspects, the present disclosure relates to a vector comprising the nucleic acid of any of the preceding aspects. In some aspects, the present disclosure relates to an isolated host cell comprising the nucleic acid of any of the preceding aspects or the vector of any of the preceding aspects. In some aspects, the present disclosure relates to an isolated host cell comprising (i) a nucleic acid comprising a nucleic acid sequence encoding the VH of an anti-CD300LB antibody of any of the preceding aspects and (ii) a nucleic acid comprising a nucleic acid sequence encoding the VL of the anti-CD300LB antibody.

In another aspect, the present disclosure relates to a method of producing an antibody that binds to human CD300LB, comprises culturing the host cell of any of the preceding aspects so that the anti-CD300LB antibody is produced. In certain aspects, the method further comprises recovering the anti-CD300LB antibody produced by the cell.

In another aspect, the present disclosure relates to a pharmaceutical composition comprises an anti-CD300LB antibody of any one of the preceding aspects and a pharmaceutically acceptable carrier.

In one aspect, the present disclosure relates to a method of detecting CD300LB in a sample comprising contacting said sample with an anti-CD300LB antibody of any of the preceding aspects, optionally wherein the method further comprises detecting the binding of the antibody to CD300LB in the sample.

It is to be understood that one, some, or all of the properties of the various aspects described herein may be combined to form other aspects of the present disclosure. These and other aspects of the disclosure will become apparent to one of skill in the art. These and other aspects of the disclosure are further described by the detailed description that follows.

### BRIEF DESCRIPTION OF THE DRAWINGS

FIG. 1 sets forth data showing anti-CD300LB antibody binding to human CD300LB-expressing Jurkat cells.

FIG. 2 sets forth data showing anti-CD300LB antibody binding to human CD300LB-expressing FreeStyle HEK cells.

FIG. 3 sets forth data showing anti-CD300LB antibody binding to parental FreeStyle HEK cells.

FIG. 4 sets forth data showing agonistic activity of anti-CD300LB antibodies in Jurkat cells expressing recombinant human CD300LB.

FIG. 5 sets forth data showing agonistic activity of anti-CD300LB antibodies in BaF/3 cells expressing recombinant murine CD300LB.

FIG. 6 sets forth data showing agonistic activity of anti-CD300LB antibodies in BaF/3 cells expressing recombinant murine CD300LB.

FIG. 7A and FIG. 7B set forth data showing the dose-dependent effect of anti-CD300LB antibodies on Syk phosphorylation.

FIG. 8 sets forth data showing anti-CD300LB antibodies of the present disclosure induce DAP12 phosphorylation in cells.

### DETAILED DESCRIPTION OF THE PRESENT DISCLOSURE

The present disclosure relates to anti-CD300LB antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al., eds., (2003); Monoclonal Antibodies: A Practical Approach (P. Shepherd and C. Dean, eds., Oxford University Press, 2000).

### I. Definitions

The terms “CD300LB” or “CD300LB polypeptide” or “CD300LB protein” are used interchangeably herein refer herein to any native CD300LB from any vertebrate source, including mammals such as primates (e.g., humans and cynomolgus monkeys (cynos)) and rodents (e.g., mice and rats), unless otherwise indicated. CD300LB is also referred to as CD300B, triggering receptor expressed on myeloid cells 5 (TREM-5), immune receptor expressed on myeloid cells 3 (IREM-3), CRMF35-like molecule, leukocyte mono-Ig-like receptor 5, and CLM7. In some aspects, the term encompasses both wild-type sequences and naturally occurring variant sequences, e.g., splice variants or allelic variants. In some aspects, the term encompasses “full-length,” unprocessed CD300LB as well as any form of CD300LB that results from processing in the cell. In some aspects, the CD300LB is human CD300LB. As used herein the term “human CD300LB” refers to a polypeptide with the amino acid sequence of SEQ ID NO:127.

The terms “anti-CD300LB antibody,” “CD300LB antibody,” an “antibody that binds to CD300LB,” and “antibody that specifically binds CD300LB” refer to an antibody that is capable of binding CD300LB with sufficient affinity such that the antibody is useful as a diagnostic and/or therapeutic agent in targeting CD300LB. In one aspect, the extent of binding of an anti-CD300LB antibody to an unrelated, non-CD300LB polypeptide is less than about 10% of the binding of the antibody to CD300LB as measured, e.g., by a radioimmunoassay (RIA). In certain aspects, an antibody that binds to CD300LB has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain aspects, an anti-CD300LB antibody binds to an epitope of CD300LB that is conserved among CD300LB from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antigen-binding antibody fragments so long as they exhibit the desired biological activity.

“Native antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-CD300LB antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-CD300LB antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-CD300LB antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure can be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypeptide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-CD300LB antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibodyfragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-CD300LB antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

The term “diabodies” refers to small antibody fragments prepared by constructing sFv fragments (see preceding paragraph) with short linkers (about 5-10) residues) between the VH and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the VH and V_{L} domains of the two antibodies are present on different polypeptide chains.

As used herein, a “chimeric antibody” refers to an antibody (immunoglobulin), such as a chimeric anti-CD300LB antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-CD300LB antibodies of the present disclosure, are chimeric antibodies comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-CD300LB antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-CD300LB antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some aspects, the HVRs may be Kabat complementarity-determining regions (CDRs) based on sequence variability and are the most commonly used (Kabat et al., supra). In some aspects, the HVRs may be Chothia CDRs. Chothia refers instead to the location of the structural loops (Chothia and Lesk J Mol. Biol. 196:901-917 (1987)). In some aspects, the HVRs may be AbM HVRs. The AbM HVRs represent a compromise between the Kabat CDRs and Chothia structural loops, and are used by Oxford Molecular's AbM antibody-modeling software. In some aspects, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred aspect) (H2), and 93-102, 94-102, or 95-102 (H3) in the V_{H}. The variable-domain residues are numbered according to Kabat et al., supra, for each of these extended-HVR definitions.

“Framework” or “FR” residues are those variable-domain residues other than the HVR residues as herein defined.

An “acceptor humanframework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some aspects, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one aspect, the VL acceptor human framework is identical in sequence to the V_{L} human immunoglobulin framework sequence or human consensus framework sequence.

A “human consensusframework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-CD300LB antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least 80% homology with a native sequence Fc region and/or with an Fc region of a parent polypeptide, and most preferably at least 90% homology therewith, more preferably at least 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies that compete for the same epitope or overlapping epitopes means competition between antibody as determined by an assay in which the antibody being tested prevents or inhibits (e.g., reduces) specific binding of a reference molecule (e.g., a ligand, or a reference antibody) to a common antigen (e.g., CD300LB or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a CD300LB polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some aspects, the interaction can be inhibited by at least any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of polypeptides and/or macromolecules.

An “isolated” antibody, such as an isolated anti-CD300LB antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred aspects, the antibody will be purified: (1) to greater than 95% by weight of antibody as determined by, for example, the Lowry method, and in some aspects, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-CD300LB antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some aspects, the individual is human.

The term “about” as used herein refers to the usual error range for the respective value readily known to the skilled person in this technical field. In some aspects when “about” is used to modify a numeric value or numeric range, the term indicates that deviations of up t 10% above and down to 10% below the value or range remain within the intended meaning of the recited value or range. Reference to “about” a value or parameter herein includes (and describes) aspects that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspects of the present disclosure described herein include “comprising,” “consisting,” and “consisting essentially of” aspects.

### I. Anti-CD300LB Antibodies

Provided herein are anti-CD300LB antibodies. Antibodies provided herein are useful, e.g., for the diagnosis or treatment of the CD300LB associated disorders.

In one aspect, the present disclosure provides isolated (e.g., monoclonal) antibodies that bind to an epitope within a CD300LB protein or polypeptide of the present disclosure. CD300LB proteins or polypeptides of the present disclosure include, without limitation, a mammalian CD300LB protein or polypeptide, human CD300LB protein or polypeptide (SEQ ID NO: 127), mouse (murine) CD300LB protein or polypeptide (SEQ ID NO: 128), and cynomolgus (cyno) CD300LB protein or polypeptide (SEQ ID NO: 129). CD300LB proteins and polypeptides of the present disclosure include naturally occurring variants of CD300LB. In some aspects, CD300LB proteins and polypeptides of the present disclosure are membrane bound. In some aspects, CD300LB proteins and polypeptides of the present disclosure are a soluble extracellular domain of CD300LB.

In some aspects, CD300LB is expressed in a cell. In some aspects, CD300LB is expressed in myeloid cells, including without limitation, macrophages, dendritic cells, or microglia. In some aspects, CD300LB is expressed in microglia.

CD300LB ligands

CD300LB proteins of the present disclosure interact with (e.g., bind) one or more ligands, including, the lipids phosphatidylserine and phytoshingosine. During apoptosis, phosphatidylserine is exposed to the outer leaflet of the cell membrane. CD300LB regulates the phagocytosis of apoptotic cells via phosphatidylserine binding.

Anti-CD300LB antibodies of the present disclosure do not block or inhibit binding of phosphatidylserine and/or of phytoshingosine binding to CD300LB. Accordingly, in some aspects, an anti-CD300LB antibody of the present disclosure does not inhibit or reduce binding between CD300LB and one or more CD300LB ligands.

### Efferocytosis

Efferocytosis refers to phagocytic clearance of dying or apoptotic cells. Efferocytosis can be accomplished by professional phagocytes (e.g., macrophages, dendritic cells, microglia), non-professional phagocytes (e.g., epithelial cells, fibroblasts, retinal pigment epithelial cells), or specialized phagocytes. (Elliott et al, 2017, J Immunol, 198:1387-1394.) Efferocytosis leads to the removal of dead or dying cells before their membrane integrity is breached and their cellular contents leak into the surrounding tissue, thus preventing exposure of tissue to toxic enzymes, oxidants, and other intracellular components.

Apoptotic cells expose a variety of molecules on their cell surface (“eat-me” signals) that are recognized by receptors on phagocytic cells. One such “eat me” signaling molecules is phosphatidylserine, which is normally confined to the inner leaflet of the cell membrane. During apoptosis, phosphatidylserine is exposed to the outer leaflet of the cell membrane. CD300LB regulates the phagocytosis of apoptotic cells via phosphatidylserine binding; such ligand engagement with CD300LB activates efferocytosis (Murakami et al, 2014, Cell Death Differ, 21:1746-1757; Voss et al, 2015, Mol Cell Oncol, 2:e964625). Accordingly, agonistic anti-CD300LB antibodies of the present disclosure are capable of increasing efferocytosis by phagocytic cells (e.g, microglia).

The ability of an antibody to block (or not block) efferocytosis can be determined, e.g., using the methods available to one of skill in the art. For instance, an efferocytosis assay can comprise (i) adding apoptotic cells to phagocytic cells that have been exposed or not exposed to an antibody or exposed to a test antibody and a negative control antibody and (ii) determining the uptake of the apoptoic cells by the phagocytic cells. The phagocytic cells can be professional phagocytes or non-professional phagocytes as discussed above. In some aspects, the phagocytic cells are macrophages. In some aspects, the phagocytic cells (e.g., macrophages) are starved (e.g., for about an hour) prior to the exposure to the antibody and/or the apoptotic cells. In some aspects, the phagocytic cells (e.g., macrophages) are incubated with the antibody for about 5 minutes to about an hour (e.g., for about 30 minutes) prior to the exposure to the apoptotic cells, e.g., at about 37° C. The apoptotic cells can be, e.g., Jurkat cells that were treated with an apoptosis-inducing agent such as 1 M staurosporin (SigmaAldrich). The apoptotic cells can be labeled cells (e.g., dyed cells). In some aspects, the apoptotic cells are exposed to the phagocytic cells (e.g., macrophages) for about an hour. An antibody that does not block efferocytosis does not significantly increase the uptake of apoptotic cells in such an assay as compared to the uptake in the absence of the antibody or in the presence of a negative control antibody.

### Phagocytosis

Phagocytosis refers to the process by which phagocytes ingest or engulf apoptotic cells, particles, or cell debris. Within the central nervous system, phagocytosis is a critical process required for proper neural circuit development and maintaining homeostasis. Destruction of myelin sheathes within the CNS, as occurs in multiple sclerosis, produces degenerating myelin at site of injury and inflammation. The resulting myelin debris must be cleared through phagocytosis from sites of injury to promote repair.

The ability of an antibody to increase phagocytosis can be determined using assays known in the art (see e.g., Healy et al, 2016, J Immunol, 196:3375-3384). For example, a phagocytosis assay can comprise (i) adding myelin to cells (e.g. myeloid cells) in the presence and the absence of an antibody or in the presence of the test antibody and a negative control antibody, and (ii) determining the uptake of the myelin by the cells. The cells can be plated cells (e.g., plated myeloid cells). The cells (e.g. myeloid cells) can be polarized. (See e.g., Durafourt et al, 2012, Glia 60:717-727.) The myelin can be labeled. For example, the myelin can be dyed using, e.g., a pH-sensitive dye such as ph-Rhodamine (Invitrogen). In order to obtain dyed myelin, myelin and a dye (e.g., a pH-sensitive dye) can be incubated, e.g., for about 1 hour, optionally in PBS. The myelin (e.g. dyed myelin) can be added to the cells (e.g., myeloid cells) to a final concentration of, e.g., about 5 μg/ml to about 20 μg/ml. Thus, in some aspects, myelin (e.g. dyed myelin) is added to the cells (e.g., myeloid cells) to a final concentration of about 5 μg/ml or about 20 μg/ml. An antibody that increases phagocytosis of myelin (i.e., increases clearance of myelin by phagocytosis) increases the uptake of myelin by cells in such an assay as compared to the uptake in the absence of the antibody or in the presence of a negative control antibody.

Syk phosphorylation

Spleen tyrosine kinase (Syk) phosphorylation signaling is a signal transduction pathway associated with various cell functions and plays a critical role in immune cell function. Upon immune stimulation, Syk is able to bind specific phosphorylation motifs in ITAMs. Anti-CD300LB antibodies of the present disclosure increased Syk phosphorylation levels in cells in vitro.

### Epitope Binning

Epitope binning is a competitive immunoassay used to characterize and sort a library of monoclonal antibodies against a target protein (Abdiche et al, 2009, Analytical Biochemistry, 386:172-180). Epitope binning is also referred to as epitope mapping and epitope characterization (Brooks, 2014, Current Drug Discovery Technology, 11:109-112). Antibodies against a particular target (e.g., CD300LB) are tested against all other antibodies in the library in a pairwise fashion to determine if any of the antibodies block one another's binding to an epitope of the target. After each antibody has a profile created against all of the other antibodies of the library, a competitive antibody blocking profile is created for each antibody relative to the other antibodies in the library. Closely related binning profiles indicate that the antibodies have the same or a closely related (e.g., overlapping) epitope and are “binned” together.

As shown in the Examples below, anti-CD300LB antibodies of the present disclosure displayed a variety of binning profiles, characterized by bin 1a, bin 1b, bin 1c, bin 1d, bin 1e, and bin 2. Bin 1 anti-CD300LB antibodies of the present disclosure (bin 1a, bin 1b, bin 1c, bin 1d, and bin 1e) are capable of at least partially blocking binding of other bin 1 anti-CD300LB antibodies to human CD300LB.

### A. Exemplary Antibodies and Certain Other Antibody Aspects

In some aspects, provided herein are anti-CD300LB antibodies comprising at least one, two, three, four, five, or six HVRs selected from: (a) HVR-H1 comprising an amino acid sequence selected the group consisting of SEQ ID NOs:36, 37, 38, 39, 40, 41, 42, 43, 44, 45, and 46; (b) HVR-H2 comprising an amino acid sequence selected from the group consisting of SEQ ID NOs:47, 48, 49, 50, 51, 52, 53, 54, 55, 56, 57, 58, 59, 60, 61, 62, 63, and 64; (c) HVR-H3 comprising an amino acid sequence selected from the group consisting of SEQ ID NOs:65, 66, 67, 68, 69, 70, 71, 72, 73, 74, 75, 76, 77, 78, and 79; (d) HVR-L1 comprising an amino acid sequence selected from the group consisting of SEQ ID NOs:80, 81, 82, 83, 84, 85, 86, 87, 88, 89, 90, 91, 92, 93, 94, 95, and 96; (e) HVR-L2 comprising an amino acid sequence selected from the group consisting of SEQ ID NOs:97, 98, 99, 100, 101, 102, 103, 104, 105, 106, 107, 108, 109, 110, and 111; and (f) HVR-L3 comprising an amino acid sequence selected from the group consisting of SEQ ID NOs:112, 113, 114, 115, 116, 117, 118, 119, 120, 121, 122, 123, 124, 125, and 126.

In some aspects, provided herein are anti-CD300LB antibodies comprising at least one, at least two, or all three V_{H} HVR sequences selected from (a) HVR-H1 comprising an amino acid sequence selected from the group consisting of SEQ ID NOs: 36, 37, 38, 39, 40, 41, 42, 43, 44, 45, and 46; (b) HVR-H2 comprising an amino acid sequence selected from the group consisting of SEQ ID NOs: 47, 48, 49, 50, 51, 52, 53, 54, 55, 56, 57, 58, 59, 60, 61, 62, 63, and 64, and (c) HVR-H3 comprising an amino acid sequence selected from the group consisting of SEQ ID NOs: 65, 66, 67, 68, 69, 70, 71, 72, 73, 74, 75, 76, 77, 78, and 79.

In some aspects, provided herein are anti-CD300LB antibodies comprising at least one, at least two, or all three V_{L} HVR sequences selected from (a) HVR-L1 comprising an amino acid sequence selected from the group consisting of SEQ ID NOs: 80, 81, 82, 83, 84, 85, 86, 87, 88, 89, 90, 91, 92, 93, 94, 95, and 96; (b) HVR-L2 comprising an amino acid sequence selected from the group consisting of SEQ ID NOs: 97, 98, 99, 100, 101, 102, 103, 104, 105, 106, 107, 108, 109, 110, and 111; and (c) HVR-L3 comprising an amino acid sequence selected from the group consisting of SEQ ID NOs: 112, 113, 114, 115, 116, 117, 118, 119, 120, 121, 122, 123, 124, 125, and 126.

In some aspects, provided herein are anti-CD300LB antibodies comprising (a) a V_{H} domain comprising at least one, at least two, or all three V_{H} HVR sequences selected from (i) HVR-H1 comprising an amino acid sequence selected from the group consisting of SEQ ID NOs: 36, 37, 38, 39, 40, 41, 42, 43, 44, 45, and 46; (ii) HVR-H2 comprising an amino acid sequence selected from the group consisting of SEQ ID NOs: 47, 48, 49, 50, 51, 52, 53, 54, 55, 56, 57, 58, 59, 60, 61, 62, 63, and 64, and (iii) HVR-H3 comprising an amino acid sequence selected from the group consisting of SEQ ID NOs: 65, 66, 67, 68, 69, 70, 71, 72, 73, 74, 75, 76, 77, 78, and 79, and (b) a V_{L} domain comprising at least one, at least two, or all three V_{L} HVR sequences selected from (i) HVR-L1 comprising an amino acid sequence selected from the group consisting of SEQ ID NOs: 80, 81, 82, 83, 84, 85, 86, 87, 88, 89, 90, 91, 92, 93, 94, 95, and 96; (ii) HVR-L2 comprising an amino acid sequence selected from the group consisting of SEQ ID NOs: 97, 98, 99, 100, 101, 102, 103, 104, 105, 106, 107, 108, 109, 110, and 111; and (iii) HVR-L3 comprising an amino acid sequence selected from the group consisting of SEQ ID NOs: 112, 113, 114, 115, 116, 117, 118, 119, 120, 121, 122, 123, 124, 125, and 126.

In some aspects, provided herein are anti-CD300B antibodies comprising: (a) HVR-H1 comprising the amino acid sequence of SEQ ID NO:36; (b) HVR-H2 comprising the amino acid sequence of SEQ ID NO:47; (c) HVR-H3 comprising the amino acid sequence of SEQ ID NO:65; (d) HVR-L1 comprising the amino acid sequence of SEQ ID NO:80; (e) HVR-L2 comprising the amino acid sequence of SEQ ID NO:97; and (f) HVR-L3 comprising the amino acid sequence of SEQ ID NO: 112; (a) HVR-H1 comprising the amino acid sequence of SEQ ID NO:37; (b) HVR-H2 comprising the amino acid sequence of SEQ ID NO:48; (c) HVR-H3 comprising the amino acid sequence of SEQ ID NO:66; (d) HVR-L1 comprising the amino acid sequence of SEQ ID NO:81; (e) HVR-L2 comprising the amino acid sequence of SEQ ID NO:98; and (f) HVR-L3 comprising the amino acid sequence of SEQ ID NO: 113; (a) HVR-H1 comprising the amino acid sequence of SEQ ID NO:38; (b) HVR-H2 comprising the amino acid sequence of SEQ ID NO:49; (c) HVR-H3 comprising the amino acid sequence of SEQ ID NO:67; (d) HVR-L1 comprising the amino acid sequence of SEQ ID NO:82; (e) HVR-L2 comprising the amino acid sequence of SEQ ID NO:99; and (f) HVR-L3 comprising the amino acid sequence of SEQ ID NO: 114; (a) HVR-H1 comprising the amino acid sequence of SEQ ID NO:36; (b) HVR-H2 comprising the amino acid sequence of SEQ ID NO:50; (c) HVR-H3 comprising the amino acid sequence of SEQ ID NO:68; (d) HVR-L1 comprising the amino acid sequence of SEQ ID NO:83; (e) HVR-L2 comprising the amino acid sequence of SEQ ID NO: 100; and (f) HVR-L3 comprising the amino acid sequence of SEQ ID NO: 112; (a) HVR-H1 comprising the amino acid sequence of SEQ ID NO:39; (b) HVR-H2 comprising the amino acid sequence of SEQ ID NO:51; (c) HVR-H3 comprising the amino acid sequence of SEQ ID NO:69; (d) HVR-L1 comprising the amino acid sequence of SEQ ID NO:84; (e) HVR-L2 comprising the amino acid sequence of SEQ ID NO:101; and (f) HVR-L3 comprising the amino acid sequence of SEQ ID NO: 115; (a) HVR-H1 comprising the amino acid sequence of SEQ ID NO:40; (b) HVR-H2 comprising the amino acid sequence of SEQ ID NO:52; (c) HVR-H3 comprising the amino acid sequence of SEQ ID NO:70; (d) HVR-L1 comprising the amino acid sequence of SEQ ID NO:85; (e) HVR-L2 comprising the amino acid sequence of SEQ ID NO: 102; and (f) HVR-L3 comprising the amino acid sequence of SEQ ID NO: 116; (a) HVR-H1 comprising the amino acid sequence of SEQ ID NO:41; (b) HVR-H2 comprising the amino acid sequence of SEQ ID NO:53; (c) HVR-H3 comprising the amino acid sequence of SEQ ID NO:71; (d) HVR-L1 comprising the amino acid sequence of SEQ ID NO:86; (e) HVR-L2 comprising the amino acid sequence of SEQ ID NO: 103; and (f) HVR-L3 comprising the amino acid sequence of SEQ ID NO: 117; (a) HVR-H1 comprising the amino acid sequence of SEQ ID NO:42; (b) HVR-H2 comprising the amino acid sequence of SEQ ID NO:54; (c) HVR-H3 comprising the amino acid sequence of SEQ ID NO:72; (d) HVR-L1 comprising the amino acid sequence of SEQ ID NO:87; (e) HVR-L2 comprising the amino acid sequence of SEQ ID NO: 104; and (f) HVR-L3 comprising the amino acid sequence of SEQ ID NO: 118; (a) HVR-H1 comprising the amino acid sequence of SEQ ID NO:40; (b) HVR-H2 comprising the amino acid sequence of SEQ ID NO:55; (c) HVR-H3 comprising the amino acid sequence of SEQ ID NO:70; (d) HVR-L1 comprising the amino acid sequence of SEQ ID NO:85; (e) HVR-L2 comprising the amino acid sequence of SEQ ID NO: 102; and (f) HVR-L3 comprising the amino acid sequence of SEQ ID NO: 116; (a) HVR-H1 comprising the amino acid sequence of SEQ ID NO:43; (b) HVR-H2 comprising the amino acid sequence of SEQ ID NO:56; (c) HVR-H3 comprising the amino acid sequence of SEQ ID NO:73; (d) HVR-L1 comprising the amino acid sequence of SEQ ID NO:88; (e) HVR-L2 comprising the amino acid sequence of SEQ ID NO: 105; and (f) HVR-L3 comprising the amino acid sequence of SEQ ID NO: 119; (a) HVR-H1 comprising the amino acid sequence of SEQ ID NO:44; (b) HVR-H2 comprising the amino acid sequence of SEQ ID NO:57; (c) HVR-H3 comprising the amino acid sequence of SEQ ID NO:74; (d) HVR-L1 comprising the amino acid sequence of SEQ ID NO:89; (e) HVR-L2 comprising the amino acid sequence of SEQ ID NO: 106; and (f) HVR-L3 comprising the amino acid sequence of SEQ ID NO: 120; (a) HVR-H1 comprising the amino acid sequence of SEQ ID NO:44; (b) HVR-H2 comprising the amino acid sequence of SEQ ID NO:58; (c) HVR-H3 comprising the amino acid sequence of SEQ ID NO:75; (d) HVR-L1 comprising the amino acid sequence of SEQ ID NO:90; (e) HVR-L2 comprising the amino acid sequence of SEQ ID NO: 107; and (f) HVR-L3 comprising the amino acid sequence of SEQ ID NO: 121; (a) HVR-H1 comprising the amino acid sequence of SEQ ID NO:44; (b) HVR-H2 comprising the amino acid sequence of SEQ ID NO:59; (c) HVR-H3 comprising the amino acid sequence of SEQ ID NO:75; (d) HVR-L1 comprising the amino acid sequence of SEQ ID NO:91; (e) HVR-L2 comprising the amino acid sequence of SEQ ID NO: 108; and (f) HVR-L3 comprising the amino acid sequence of SEQ ID NO: 122; (a) HVR-H1 comprising the amino acid sequence of SEQ ID NO:45; (b) HVR-H2 comprising the amino acid sequence of SEQ ID NO:60; (c) HVR-H3 comprising the amino acid sequence of SEQ ID NO:76; (d) HVR-L1 comprising the amino acid sequence of SEQ ID NO:92; (e) HVR-L2 comprising the amino acid sequence of SEQ ID NO: 109; and (f) HVR-L3 comprising the amino acid sequence of SEQ ID NO: 123; (a) HVR-H1 comprising the amino acid sequence of SEQ ID NO:44; (b) HVR-H2 comprising the amino acid sequence of SEQ ID NO:61; (c) HVR-H3 comprising the amino acid sequence of SEQ ID NO:77; (d) HVR-L1 comprising the amino acid sequence of SEQ ID NO:93; (e) HVR-L2 comprising the amino acid sequence of SEQ ID NO:110; and (f) HVR-L3 comprising the amino acid sequence of SEQ ID NO: 121; (a) HVR-H1 comprising the amino acid sequence of SEQ ID NO:44; (b) HVR-H2 comprising the amino acid sequence of SEQ ID NO:62; (c) HVR-H3 comprising the amino acid sequence of SEQ ID NO:77; (d) HVR-L1 comprising the amino acid sequence of SEQ ID NO:94; (e) HVR-L2 comprising the amino acid sequence of SEQ ID NO:110; and (f) HVR-L3 comprising the amino acid sequence of SEQ ID NO: 124; (a) HVR-H1 comprising the amino acid sequence of SEQ ID NO:44; (b) HVR-H2 comprising the amino acid sequence of SEQ ID NO:63; (c) HVR-H3 comprising the amino acid sequence of SEQ ID NO:78; (d) HVR-L1 comprising the amino acid sequence of SEQ ID NO:95; (e) HVR-L2 comprising the amino acid sequence of SEQ ID NO: 107; and (f) HVR-L3 comprising the amino acid sequence of SEQ ID NO: 125; and (a) HVR-H1 comprising the amino acid sequence of SEQ ID NO:46; (b) HVR-H2 comprising the amino acid sequence of SEQ ID NO:64; (c) HVR-H3 comprising the amino acid sequence of SEQ ID NO:79; (d) HVR-L1 comprising the amino acid sequence of SEQ ID NO:96; (e) HVR-L2 comprising the amino acid sequence of SEQ ID NO:111; and (f) HVR-L3 comprising the amino acid sequence of SEQ ID NO: 126.

In another aspect, an anti-CD300LB antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs: 1, 3, 5, 7, 9, 11, 13, 15, 17, 18, 20, 22, 24, 26, 28, 30, 32, and 34. In certain aspects, a V_{H} sequence having at least 90%, 91%, 92%, 93%, 94%, 95%, 96%, 97%, 98%, or 99% identity to an amino acid sequence selected from the group consisting of SEQ ID NOs: 1, 3, 5, 7, 9, 11, 13, 15, 17, 18, 20, 22, 24, 26, 28, 30, 32, and 34 contains substitutions (e.g., conservative substitutions), insertions, or deletions relative to the reference sequence, but an anti-CD300LB antibody comprising that sequence retains the ability to bind to CD300LB. In certain aspects, a total of 1 to 10 amino acids have been substituted, inserted, and/or deleted in SEQ ID NO: 1, 3, 5, 7, 9, 11, 13, 15, 17, 18, 20, 22, 24, 26, 28, 30, 32, or 34. In certain aspects, a total of 1 to 5 amino acids have been substituted, inserted and/or deleted in SEQ ID NO: 210, or 222. In certain aspects, substitutions, insertions, or deletions occur in regions outside the HVRs (i.e., in the FRs). Optionally, the anti-CD300LB antibody comprises the V_{H} sequence of SEQ ID NO: 1, 3, 5, 7, 9, 11, 13, 15, 17, 18, 20, 22, 24, 26, 28, 30, 32, or 34, including post-translational modifications of that sequence. In a particular aspect, the V_{H} comprises one, two or three HVRs selected from: (a) HVR-H1 comprising an amino acid sequence selected from the group consisting of SEQ ID NOs: 36, 37, 38, 39, 40, 41, 42, 43, 44, 45, and 46; (b) HVR-H2 comprising an amino acid sequence selected from the group consisting of SEQ ID NOs: 47, 48, 49, 50, 51, 52, 53, 54, 55, 56, 57, 58, 59, 60, 61, 62, 63, and 64; and (c) HVR-H3 comprising an amino acid sequence selected from the group consisting of SEQ ID NOs: 65, 66, 67, 68, 69, 70, 71, 72, 73, 74, 75, 76, 77, 78, and 79.

In another aspect, an anti-CD300LB antibody is provided, wherein the antibody comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs: 2, 4, 6, 8, 10, 12, 14, 16, 19, 21, 23, 25, 27, 29, 31, 33, and 35. In certain aspects, a V_{L} sequence having at least 90%, 91%, 92%, 93%, 94%, 95%, 96%, 97%, 98%, or 99% identity to an amino acid sequence selected from the group consisting of SEQ ID NOs: 2, 4, 6, 8, 10, 12, 14, 16, 19, 21, 23, 25, 27, 29, 31, 33, and 35, and contains substitutions (e.g., conservative substitutions), insertions, or deletions relative to the reference sequence, but an anti-CD300LB antibody comprising that sequence retains the ability to bind to CD300LB. In some aspects, a total of 1 to 10 amino acids have been substituted, inserted and/or deleted in SEQ ID NO: 2, 4, 6, 8, 10, 12, 14, 16, 19, 21, 23, 25, 27, 29, 31, 33, or 35. In certain aspects, a total of 1 to 5 amino acids have been substituted, inserted and/or deleted in SEQ ID NO: 2, 4, 6, 8, 10, 12, 14, 16, 19, 21, 23, 25, 27, 29, 31, 33, or 35. In certain aspects, the substitutions, insertions, or deletions occur in regions outside the HVRs (i.e., in the FRs). Optionally, the anti-CD300LB antibody comprises the V_{L} sequence of SEQ ID NO: 2, 4, 6, 8, 10, 12, 14, 16, 19, 21, 23, 25, 27, 29, 31, 33, or 35, including post-translational modifications of that sequence. In a particular aspect, the V_{L} comprises one, two or three HVRs selected from (a) HVR-L1 comprising an amino acid sequence selected from the group consisting of SEQ ID NOs: 80, 81, 82, 83, 84, 85, 86, 87, 88, 89, 90, 91, 92, 93, 94, 95, and 96; (b) HVR-L2 comprising an amino acid sequence selected from the group consisting of SEQ ID NOs: 97, 98, 99, 100, 101, 102, 103, 104, 105, 106, 107, 108, 109, 110, and 111; and (c) HVR-L3 comprising an amino acid sequence selected from the group consisting of SEQ ID NOs: 112, 113, 114, 115, 116, 117, 118, 119, 120, 121, 122, 123, 124, 125, and 126.

In some aspects, an anti-CD300LB antibody is provided, wherein the antibody comprises a V_{H} as in any of the aspects provided above, and a V_{L} as in any of the aspects provided above. In some aspects, provided herein are anti-CD300LB antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs: 1, 3, 5, 7, 9, 11, 13, 15, 17, 18, 20, 22, 24, 26, 28, 30, 32, and 34, and SEQ ID NOs: 2, 4, 6, 8, 10, 12, 14, 16, 19, 21, 23, 25, 27, 29, 31, 33, and 35, respectively, including post-translational modifications of those sequences.

In some aspects, provided herein are anti-CD300LB antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:1 and V_{L} comprising the amino acid sequence of SEQ ID NO:2; V_{H} comprising the amino acid sequence of SEQ ID NO:3 and V_{L} comprising the amino acid sequence of SEQ ID NO:4; V_{H} comprising the amino acid sequence of SEQ ID NO:5 and V_{L} comprising the amino acid sequence of SEQ ID NO:6; V_{H} comprising the amino acid sequence of SEQ ID NO:7 and V_{L} comprising the amino acid sequence of SEQ ID NO:8; V_{H} comprising the amino acid sequence of SEQ ID NO:9 and V_{L} comprising the amino acid sequence of SEQ ID NO:10; V_{H} comprising the amino acid sequence of SEQ ID NO: 11 and V_{L} comprising the amino acid sequence of SEQ ID NO: 12; V_{H} comprising the amino acid sequence of SEQ ID NO: 13 and V_{L} comprising the amino acid sequence of SEQ ID NO: 14; V_{H} comprising the amino acid sequence of SEQ ID NO: 15 and V_{L} comprising the amino acid sequence of SEQ ID NO:16; V_{H} comprising the amino acid sequence of SEQ ID NO: 17 and V_{L} comprising the amino acid sequence of SEQ ID NO: 12; V_{H} comprising the amino acid sequence of SEQ ID NO: 18 and V_{L} comprising the amino acid sequence of SEQ ID NO: 19; V_{H} comprising the amino acid sequence of SEQ ID NO:20 and V_{L} comprising the amino acid sequence of SEQ ID NO:21; V_{H} comprising the amino acid sequence of SEQ ID NO:22 and V_{L} comprising the amino acid sequence of SEQ ID NO:23; V_{H} comprising the amino acid sequence of SEQ ID NO:24 and V_{L} comprising the amino acid sequence of SEQ ID NO:25; V_{H} comprising the amino acid sequence of SEQ ID NO:26 and V_{L} comprising the amino acid sequence of SEQ ID NO:27; V_{H} comprising the amino acid sequence of SEQ ID NO:28 and V_{L} comprising the amino acid sequence of SEQ ID NO:29; V_{H} comprising the amino acid sequence of SEQ ID NO:30 and V_{L} comprising the amino acid sequence of SEQ ID NO:31; V_{H} comprising the amino acid sequence of SEQ ID NO:32 and V_{L} comprising the amino acid sequence of SEQ ID NO:33; and V_{H} comprising the amino acid sequence of SEQ ID NO:34 and V_{L} comprising the amino acid sequence of SEQ ID NO:35.

In some aspects, an anti-CD300LB antibody of the present disclosure competitively inhibits binding of at least one reference antibody selected from anti-CD300LB antibody CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18 and any combination thereof, for binding to CD300LB.

In some aspects, an anti-CD300LB antibody of the present disclosure binds to an epitope of human CD300LB that is the same as or overlaps with the CD300LB epitope bound by at least one reference antibody selected from anti-CD300LB antibody CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some aspects, an anti-CD300LB antibody of the present disclosure competitively inhibits binding of at least one reference antibody, or binds to an epitope of human CD300LB that is the same as or overlaps with the CD300LB epitope bound by at least one reference antibody, wherein the reference antibody is an anti-CD300LB antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:1 and V_{L} comprising the amino acid sequence of SEQ ID NO:2; V_{H} comprising the amino acid sequence of SEQ ID NO:3 and V_{L} comprising the amino acid sequence of SEQ ID NO:4; V_{H} comprising the amino acid sequence of SEQ ID NO:5 and V_{L} comprising the amino acid sequence of SEQ ID NO:6; V_{H} comprising the amino acid sequence of SEQ ID NO:7 and V_{L} comprising the amino acid sequence of SEQ ID NO:8; V_{H} comprising the amino acid sequence of SEQ ID NO:9 and V_{L} comprising the amino acid sequence of SEQ ID NO: 10; V_{H} comprising the amino acid sequence of SEQ ID NO: 11 and V_{L} comprising the amino acid sequence of SEQ ID NO: 12; V_{H} comprising the amino acid sequence of SEQ ID NO: 13 and V_{L} comprising the amino acid sequence of SEQ ID NO: 14; V_{H} comprising the amino acid sequence of SEQ ID NO: 15 and V_{L} comprising the amino acid sequence of SEQ ID NO: 16; V_{H} comprising the amino acid sequence of SEQ ID NO: 17 and V_{L} comprising the amino acid sequence of SEQ ID NO:12; V_{H} comprising the amino acid sequence of SEQ ID NO: 18 and V_{L} comprising the amino acid sequence of SEQ ID NO: 19; V_{H} comprising the amino acid sequence of SEQ ID NO:20 and V_{L} comprising the amino acid sequence of SEQ ID NO:21; V_{H} comprising the amino acid sequence of SEQ ID NO:22 and V_{L} comprising the amino acid sequence of SEQ ID NO:23; V_{H} comprising the amino acid sequence of SEQ ID NO:24 and V_{L} comprising the amino acid sequence of SEQ ID NO:25; V_{H} comprising the amino acid sequence of SEQ ID NO:26 and V_{L} comprising the amino acid sequence of SEQ ID NO:27; V_{H} comprising the amino acid sequence of SEQ ID NO:28 and V_{L} comprising the amino acid sequence of SEQ ID NO:29; V_{H} comprising the amino acid sequence of SEQ ID NO:30 and V_{L} comprising the amino acid sequence of SEQ ID NO:31; V_{H} comprising the amino acid sequence of SEQ ID NO:32 and V_{L} comprising the amino acid sequence of SEQ ID NO:33; and V_{H} comprising the amino acid sequence of SEQ ID NO:34 and V_{L} comprising the amino acid sequence of SEQ ID NO:35.

In some aspects, the anti-CD300LB antibody according to any of the above aspects is a monoclonal antibody, including a humanized and/or human antibody. In some aspects, the anti-CD300LB antibody is an antibody fragment, e.g., a Fv, Fab, Fab′, scFv, diabody, or F(ab′)2 fragment. In some aspects, the anti-CD300B antibody is a substantially full-length antibody, e.g., an IgG1 antibody, IgG2a antibody or other antibody class or isotype as defined herein.

### In Some Aspects, an Anti-CD300LB Antibody According to any of the Above Aspects May

incorporate any of the features, singly or in combination, as described below:
(1) Anti-CD300LB antibody binding affinity

In some aspects of any of the antibodies provided herein, the antibody has a dissociation constant (K_{D}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one aspect, Kd is measured by a radiolabeled antigen binding assay (RIA). In some aspect, an RIA is performed with the Fab version of an antibody of interest and its antigen, for example as described in Chen et al., J. Mol. Biol. 293:865-881(1999)). In some aspects, K_{D} is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some aspects, the K_{D} is determined using a monovalent antibody (e.g., a Fab) or a full-length antibody. In some aspects, the K_{D} is determined using a full-length antibody in a monovalent form.

In some aspects, an anti-CD300LB antibody of the present disclosure binds to human CD300LB, wherein the K_{D} of binding to human CD300LB is from about 15 nM to about 329 nM.

### (2) Antibody Fragments

In some aspects of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al., Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al., Nat. Med. 9:129-134 (2003). Triabodies and tetrabodies are also described in Hudson et al., Nat. Med. 9:129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some aspects of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some aspects of any of the antibodies provided herein, the antibody is a humanized antibody. Typically, a non-human antibody is humanized to reduce immunogenicity to humans, while retaining the specificity and affinity of the parental non-human antibody. In certain aspects, a humanized antibody is substantially non-immunogenic in humans. In certain aspects, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain aspects, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al., Front. Biosci. 13:1619-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### (4) Human Antibodies

In some aspects of any of the antibodies provided herein, the antibody is a human antibody. Human antibodies can be produced using various techniques known in the art. Human antibodies are described generally in van Dijk et al., Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human monoclonal antibodies with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al., J. Immunol. 147:86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al., Histology and Histopathology 20(3):927-937 (2005) and Vollmers et al., Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some aspects of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol. 12: 433-455 (1994). For example, a variety of methods are known in the art for generating phage display libraries and screening such libraries for antibodies possessing the desired binding characteristics. See also Sidhu et al., J. Mol. Biol. 338(2): 299-310, 2004; Lee et al., J. Mol. Biol. 340(5): 1073-1093, 2004; Fellouse Proc. Natl. Acad. Sci. USA 101(34):12467-12472 (2004); and Lee et al., J. Immunol. Methods 284(−2):119-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al., J Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some aspects of any of the antibodies provided herein, the antibody comprises an Fc. In some aspects, the Fc is a human IgG1, IgG2, IgG3, and/or IgG4 isotype. In some aspects, the antibody is of the IgG class, the IgM class, or the IgA class.

In certain aspects of any of the antibodies provided herein, the antibody has an IgG2 isotype. In some aspects, the antibody contains a human IgG2 constant region. In some aspects, the human IgG2 constant region includes an Fc region. In some aspects, the antibody induces the one or more CD300LB activities or independently of binding to an Fc receptor. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has an IgG1 isotype. In some aspects, the antibody contains a mouse IgG1 constant region. In some aspects, the antibody contains a human IgG1 constant region. In some aspects, the human IgG1 constant region includes an Fc region. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has an IgG4 isotype. In some aspects, the antibody contains a human IgG4 constant region. In some aspects, the human IgG4 constant region includes an Fc region. In some aspects, the antibody binds an inhibitory Fc receptor. In certain aspects, the inhibitory Fc receptor is inhibitory Fc-gamma receptor IIB (FcγIIB).

In certain aspects of any of the antibodies provided herein, the antibody has a hybrid IgG2/4 isotype. In some aspects, the antibody includes an amino acid sequence comprising amino acids 118 to 260 according to EU numbering of human IgG2 and amino acids 261-447 according to EU numbering of human IgG4 (WO 1997/11971; WO 2007/106585).

In some aspects, the Fe region increases clustering without activating complement as compared to a corresponding antibody comprising an Fc region that does not comprise the amino acid substitutions. In some aspects, the antibody induces one or more activities of a target specifically bound by the antibody. In some aspects, the antibody binds to CD300LB.

It may also be desirable to modify an anti-CD300LB antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some aspects, the effector function is impaired by removing N-glycosylation of the Fc region (e.g., in the CH2 domain of IgG) of the antibody. In some aspects, the effector function is impaired by modifying regions such as 233-236, 297, and/or 327-331 of human IgG as described in WO 99/58572 and Armour et al., Molecular Immunology 40: 585-593 (2003); Reddy et al., J. Immunology 164:1925-1933 (2000). In other aspects, it may also be desirable to modify an anti-CD300LB antibody of the present disclosure to modify effector function to increase finding selectivity toward the ITIM-containing FcgRIIb (CD32b) to increase clustering of CD300LB antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Antibody Variants

In some aspects of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some aspects of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain aspects, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids can be substituted for other amino acids having a similar hydropathic index or score and still retain a similar biological activity. In making changes based upon the hydropathic index, in certain aspects, the substitution of amino acids whose hydropathic indices are within +2 is included. In certain aspects, those which are within ±1 are included, and in certain aspects, those within +0.5 are included.

It is also understood in the art that the substitution of like amino acids can be made effectively on the basis of hydrophilicity, particularly where the biologically functional protein or peptide thereby created is intended for use in immunological aspects, as in the present case. In certain aspects, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain aspects, the substitution of amino acids whose hydrophilicity values are within +2 is included, in certain aspects, those which are within +1 are included, and in certain aspects, those within +0.5 are included. One can also identify epitopes from primary amino acid sequences on the basis of hydrophilicity. These regions are also referred to as “epitopic core regions”.

In certain aspects of the variant VH and VL sequences provided above, each HVR is unaltered.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue outside the HVRs and not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some aspects of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure may be made in order to create antibody variants with certain improved properties.

In one aspect, antibody variants are provided having a carbohydrate structure that lacks fucose attached (directly or indirectly) to an Fc region. See, e.g., U.S. Patent Publication Nos. 2003/0157108 and 2004/0093621. Examples of publications related to “defucosylated” or “fucose-deficient” antibody variants include: U.S. Patent Publication Nos. 2003/0157108, 2003/0115614, 2002/0164328, 2004/0093621, 2004/0132140, 2004/0110704, 2004/0110282, and US 2004/0109865; Okazaki et al., J. Mol. Biol. 336:1239-1249 (2004); Yamane-Ohnuki et al., Biotech. Bioeng. 87:614 (2004). Examples of cell lines capable of producing defucosylated antibodies include Led 3 CHO cells deficient in protein fucosylation (Ripka et al., Arch. Biochem. Biophys. 249:533-545 (1986); Patent Publication No. 2003/0157108), and knockout cell lines, such as alpha-1,6-fucosyltransferase gene, FUT8, knockout CHO cells (see, e.g., Yamane-Ohnuki et al., Biotech. Bioeng. 87: 614 (2004) and Kanda et al., Biotechnol. Bioeng. 94(4):680-688 (2006)).

(iii) Modified Constant Regions

In some aspects of any of the antibodies provided herein, the antibody Fc is an antibody Fc isotypes and/or modifications. In some aspects, the antibody Fc isotype and/or modification is capable of binding to Fc gamma receptor.

In some aspects of any of the antibodies provided herein, the modified antibody Fc is an IgG1 modified Fc. In some aspects, the IgG1 modified Fc comprises one or more modifications. For example, in some aspects, the IgG1 modified Fc comprises one or more amino acid substitutions (e.g., relative to a wild-type Fc region of the same isotype). In some aspects, the one or more amino acid substitutions are selected from N297A (Bolt S et al., (1993) Eur J Immunol 23:403-411), D265A (Shields et al., (2001) R. J. Biol. Chem. 276, 6591-6604), L234A, L235A (Hutchins et al., (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al., (1994) Transplantation 57:1537-1543. 31; Xu et al., (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some aspects of any of the IgG1 modified Fc, the Fc comprises N297A mutation according to EU numbering. In some aspects of any of the IgG1 modified Fc, the Fc comprises D265A and N297A mutations according to EU numbering. In some aspects of any of the IgG1 modified Fc, the Fc comprises D270A mutations according to EU numbering. In some aspects, the IgG1 modified Fc comprises L234A and L235A mutations according to EU numbering. In some aspects of any of the IgG1 modified Fc, the Fc comprises L234A and G237A mutations according to EU numbering. In some aspects of any of the IgG1 modified Fc, the Fc comprises L234A, L235A and G237A mutations according to EU numbering. In some aspects of any of the IgG1 modified Fc, the Fc comprises one or more (including all) of P238D, L328E, E233D, G237D, H268D, P271G and A330R mutations according to EU numbering. In some aspects of any of the IgG1 modified Fc, the Fc comprises one or more of S267E/L328F mutations according to EU numbering. In some aspects of any of the IgG1 modified Fc, the Fc comprises P238D, L328E, E233D, G237D, H268D, P271G and A330R mutations according to EU numbering. In some aspects of any of the IgG1 modified Fc, the Fc comprises P238D, L328E, G237D, H268D, P271G and A330R mutations according to EU numbering. In some aspects of any of the IgG1 modified Fc, the Fc comprises P238D, S267E, L328E, E233D, G237D, H268D, P271G and A330R mutations according to EU numbering. In some aspects of any of the IgG1 modified Fc, the Fc comprises P238D, S267E, L328E, G237D, H268D, P271G and A330R mutations according to EU numbering. In some aspects of any of the IgG1 modified Fc, the Fc comprises C226S, C229S, E233P, L234V, and L235A mutations according to EU numbering. In some aspects of any of the IgG1 modified Fc, the Fc comprises L234F, L235E, and P331S mutations according to EU numbering. In some aspects of any of the IgG1 modified Fc, the Fc comprises S267E and L328F mutations according to EU numbering. In some aspects of any of the IgG1 modified Fc, the Fc comprises N325S and L328F mutations according to EU numbering. In some aspects of any of the IgG1 modified Fc, the Fc comprises S267E mutations according to EU numbering. In some aspects of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some aspects of any of the IgG1 modified Fc, the Fc includes two or more amino acid substitutions that increase antibody clustering without activating complement as compared to a corresponding antibody having an Fc region that does not include the two or more amino acid substitutions. Accordingly, in some aspects of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some aspects, the IgG1 modified Fc comprises an amino acid substitution at positions E430G, L243A, L235A, and P331S according to EU numbering. In some aspects, the IgG1 modified Fc comprises an amino acid substitution at positions E430G and P331S according to EU numbering. In some aspects, the IgG1 modified Fc comprises an amino acid substitution at positions E430G and K322A according to EU numbering. In some aspects, the IgG1 modified Fc comprises an amino acid substitution at positions E430G, A330S, and P331S according to EU numbering. In some aspects, the IgG1 modified Fc comprises an amino acid substitution at positions E430G, K322A, A330S, and P331S according to EU numbering. In some aspects, the IgG1 modified Fc comprises an amino acid substitution at positions E430G, K322A, and A330S according to EU numbering. In some aspects, the IgG1 modified Fc comprises an amino acid substitution at positions E430G, K322A, and P331S according to EU numbering.

In some aspects of any of the IgG1 modified Fc, the IgG1 modified Fc may further comprise herein may be combined with an A330L mutation (Lazar et al., Proc Natl Acad Sci USA, 103:4005-4010 (2006)), or one or more of L234F, L235E, and/or P331S mutations (Sazinsky et al., Proc Natl Acad Sci USA, 105:20167-20172 (2008)), according to the EU numbering convention, to eliminate complement activation. In some aspects of any of the IgG1 modified Fc, the IgG1 modified Fc may further comprise one or more of A330L, A330S, L234F, L235E, and/or P331S according to EU numbering. In some aspects of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some aspects of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al., Cancer Cell 19:101-113 (2011); Armour at al. Immunology 40:585-593 (2003); and White et al., Cancer Cell 27:138-148 (2015)). As such, it is thought that an anti-CD300LB antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some aspects of any of the antibodies provided herein, the modified antibody Fc is an IgG2 modified Fc. In some aspects, the IgG2 modified Fc comprises one or more modifications. For example, in some aspects, the IgG2 modified Fc comprises one or more amino acid substitutions (e.g., relative to a wild-type Fc region of the same isotype). In some aspects of any of the IgG2 modified Fc, the one or more amino acid substitutions are selected from V234A (Alegre et al., Transplantation 57:1537-1543 (1994); Xu et al., Cell Immunol, 200:16-26 (2000)); G237A (Cole et al., Transplantation, 68:563-571 (1999)); H268Q, V309L, A330S, P331S (US 2007/0148167; Armour et al., Eur J Immunol 29: 2613-2624 (1999); Armour et al., The Haematology Journal 1(Suppl.1):27 (2000); Armour et al., The Haematology Journal 1(Suppl.1):27 (2000)), C219S, and/or C220S (White et al., Cancer Cell 27, 138-148 (2015)); S267E, L328F (Chu et al., Mol Immunol, 45:3926-3933 (2008)); and M252Y, S254T, and/or T256E according to the EU numbering convention. In some aspects of any of the IgG2 modified Fc, the Fc comprises an amino acid substitution at positions V234A and G237A according to EU numbering. In some aspects of any of the IgG2 modified Fc, the Fc comprises an amino acid substitution at positions C219S or C220S according to EU numbering. In some aspects of any of the IgG2 modified Fc, the Fc comprises an amino acid substitution at positions A330S and P331S according to EU numbering. In some aspects of any of the IgG2 modified Fc, the Fc comprises an amino acid substitution at positions S267E and L328F according to EU numbering.

In some aspects of any of the IgG2 modified Fc, the Fc comprises a C127S amino acid substitution according to the EU numbering convention (White et al., (2015) Cancer Cell 27, 138-148; Lightle et al., Protein Sci. 19:753-762 (2010); and WO 2008/079246). In some aspects of any of the IgG2 modified Fc, the antibody has an IgG2 isotype with a Kappa light chain constant domain that comprises a C214S amino acid substitution according to the EU numbering convention (White et al., Cancer Cell 27:138-148 (2015); Lightle et al., Protein Sci. 19:753-762 (2010); and WO 2008/079246).

In some aspects of any of the IgG2 modified Fc, the Fc comprises a C220S amino acid substitution according to the EU numbering convention. In some aspects of any of the IgG2 modified Fc, the antibody has an IgG2 isotype with a Kappa light chain constant domain that comprises a C214S amino acid substitution according to the EU numbering convention.

In some aspects of any of the IgG2 modified Fc, the Fc comprises a C219S amino acid substitution according to the EU numbering convention. In some aspects of any of the IgG2 modified Fc, the antibody has an IgG2 isotype with a Kappa light chain constant domain that comprises a C214S amino acid substitution according to the EU numbering convention.

In some aspects of any of the IgG2 modified Fe, the Fe comprises an IgG2 isotype heavy chain constant domain 1(CH1) and hinge region (White et al., Cancer Cell 27:138-148 (2015)). In certain aspects of any of the IgG2 modified Fc, the IgG2 isotype CH1 and hinge region comprise the amino acid sequence of 118-230 according to EU numbering. In some aspects of any of the IgG2 modified Fc, the antibody Fc region comprises a S267E amino acid substitution, a L328F amino acid substitution, or both, and/or a N297A or N297Q amino acid substitution according to the EU numbering convention.

In some aspects of any of the IgG2 modified Fc, the Fc further comprises one or more amino acid substitution at positions E430G, E430S, E430F, E430T, E345K, E345Q, E345R, E345Y, S440Y, and S440W according to EU numbering. In some aspects of any of the IgG2 modified Fc, the Fc may further comprise one or more mutations to enhance the antibody half-life in human serum (e.g., one or more (including all) of M252Y, S254T, and T256E mutations according to the EU numbering convention). In some aspects of any of the IgG2 modified Fc, the Fc may further comprise A330S and P331S.

In some aspects of any of the IgG2 modified Fc, the Fc is an IgG2/4 hybrid Fc. In some aspects, the IgG2/4 hybrid Fc comprises IgG2 aa 118 to 260 and IgG4 aa 261 to 447. In some aspects of any IgG2 modified Fc, the Fc comprises one or more amino acid substitutions at positions H268Q, V309L, A330S, and P331S according to EU numbering.

In some aspects of any of the IgG1 and/or IgG2 modified Fc, the Fc comprises one or more additional amino acid substitutions selected from A330L, L234F; L235E, or P331S according to EU numbering; and any combination thereof.

In certain aspects of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some aspects of any of the IgG1 and/or IgG2 modified Fc, the Fc comprises an amino acid substitution at positions E430G, L243A, L235A, and P331S according to EU numbering. In some aspects of any of the IgG1 and/or IgG2 modified Fc, the Fc comprises an amino acid substitution at positions E430G and P331S according to EU numbering. In some aspects of any of the IgG1 and/or IgG2 modified Fc, the Fc comprises an amino acid substitution at positions E430G and K322A according to EU numbering. In some aspects of any of the IgG1 and/or IgG2 modified Fc, the Fc comprises an amino acid substitution at positions E430G, A330S, and P331S according to EU numbering. In some aspects of any of the IgG1 and/or IgG2 modified Fc, the Fc comprises an amino acid substitution at positions E430G, K322A, A330S, and P331S according to EU numbering. In some aspects of any of the IgG1 and/or IgG2 modified Fc, the Fc comprises an amino acid substitution at positions E430G, K322A, and A330S according to EU numbering. In some aspects of any of the IgG1 and/or IgG2 modified Fc, the Fc comprises an amino acid substitution at positions E430G, K322A, and P331S according to EU numbering. In some aspects of any of the IgG1 and/or IgG2 modified Fc, the Fc comprises an amino acid substitution at positions S267E and L328F according to EU numbering. In some aspects of any of the IgG1 and/or IgG2 modified Fc, the Fc comprises an amino acid substitution at position C127S according to EU numbering. In some aspects of any of the IgG1 and/or IgG2 modified Fc, the Fc comprises an amino acid substitution at positions E345R, E430G and S440Y according to EU numbering.

In some aspects of any of the antibodies provided herein, the modified antibody Fc is an IgG4 modified Fc. In some aspects, the IgG4 modified Fc comprises one or more modifications. For example, in some aspects, the IgG4 modified Fc comprises one or more amino acid substitutions (e.g., relative to a wild-type Fc region of the same isotype). In some aspects of any of the IgG4 modified Fc, the one or more amino acid substitutions are selected from L235A, G237A, S229P, L236E (Reddy et al., J Immunol 164:1925-1933(2000)), S267E, E318A, L328F, M252Y, S254T, and/or T256E according to the EU numbering convention. In some aspects of any of the IgG4 modified Fc, the Fc may further comprise L235A, G237A, and E318A according to the EU numbering convention. In some aspects of any of the IgG4 modified Fc, the Fc may further comprise S228P and L235E according to the EU numbering convention. In some aspects of any of the IgG4 modified Fc, the IgG4 modified Fc may further comprise S267E and L328F according to the EU numbering convention.

In some aspects of any of the IgG4 modified Fc, the IgG4 modified Fc comprises may be combined with an S228P mutation according to the EU numbering convention (Angal et al., Mol Immunol. 30:105-108 (1993)) and/or with one or more mutations described in (Peters et al., J Biol Chem. 287(29):24525-33 (2012)) to enhance antibody stabilization.

In some aspects of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some aspects of any of the IgG4 modified Fc, the Fc comprises L235E according to EU numbering. In certain aspects of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some aspects of any of the IgG4 modified Fc, the Fc comprises an amino acid substitution at positions E430G, L243A, L235A, and P331S according to EU numbering. In some aspects of any of the IgG4 modified Fc, the Fc comprises an amino acid substitution at positions E430G and P331S according to EU numbering. In some aspects of any of the IgG4 modified Fc, the Fc comprises an amino acid substitution at positions E430G and K322A according to EU numbering. In some aspects of any of the IgG4 modified Fc, the Fc comprises an amino acid substitution at position E430 according to EU numbering. In some aspects of any of the IgG4 modified Fc, the Fc region comprises an amino acid substitution at positions E430G and K322A according to EU numbering. In some aspects of any of the IgG4 modified Fc, the Fc comprises an amino acid substitution at positions S267E and L328F according to EU numbering. In some aspects of any of the IgG4 modified Fc, the Fc comprises an amino acid substitution at position C127S according to EU numbering. In some aspects of any of the IgG4 modified Fc, the Fc comprises an amino acid substitution at positions E345R, E430G and S440Y according to EU numbering.

### (7) Other Antibody Modifications

In some aspects of any of the antibodies, the antibody is a derivative. The term “derivative” refers to a molecule that includes a chemical modification other than an insertion, deletion, or substitution of amino acids (or nucleic acids). In certain aspects, derivatives comprise covalent modifications, including, but not limited to, chemical bonding with polymers, lipids, or other organic or inorganic moieties. In certain aspects, a chemically modified antigen binding protein can have a greater circulating half-life than an antigen binding protein that is not chemically modified. In certain aspects, a chemically modified antigen binding protein can have improved targeting capacity for desired cells, tissues, and/or organs. In some aspects, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain aspects, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1,3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain aspects, a derivative is covalently modified with polyethylene glycol (PEG) subunits. In certain aspects, one or more water-soluble polymer is bonded at one or more specific position, for example at the amino terminus, of a derivative. In certain aspects, one or more water-soluble polymer is randomly attached to one or more side chains of a derivative. In certain aspects, PEG is used to improve the therapeutic capacity for an antigen binding protein. In certain aspects, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al., J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain aspects to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al., Biocoonjugate Chemistry 21 (1):5-13 (2010).

### II. Nucleic Acids, Vectors, and Host Cells

Anti-CD300LB antibodies of the present disclosure may be produced using recombinant methods and compositions, e.g., as described in U.S. Pat. No. 4,816,567. In some aspects, isolated nucleic acids having a nucleotide sequence encoding any of the anti-CD300LB antibodies of the present disclosure are provided. Such nucleic acids may encode an amino acid sequence comprising the V_{L} and/or an amino acid sequence comprising the V_{H} of the anti-CD300LB antibody (e.g., the light and/or heavy chains of the antibody). In some aspects, one or more vectors (e.g., expression vectors) comprising such nucleic acids are provided. In some aspects, a host cell comprising such nucleic acid is also provided. In some aspects, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some aspects, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-CD300LB antibody of the present disclosure are provided. In some aspects, the method comprises culturing a host cell of the present disclosure comprising a nucleic acid encoding the anti-CD300LB antibody, under conditions suitable for expression of the antibody. In some aspects, the antibody is subsequently recovered from the host cell (or host cell culture medium).

For recombinant production of an anti-CD300LB antibody of the present disclosure, a nucleic acid encoding the anti-CD300LB antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-CD300LB antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-CD300LB antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gemgross Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### III. Pharmaceutical Compositions/Formulations

Provided herein are pharmaceutical compositions and/or pharmaceutical formulations comprising the anti-CD300LB antibodies of the present disclosure and a pharmaceutically acceptable carrier.

In some aspects, pharmaceutically acceptable carrier preferably are nontoxic to recipients at the dosages and concentrations employed. The pharmaceutical compositions and/or pharmaceutical formulations to be used for in vivo administration can be sterile. This is readily accomplished by filtration through e.g., sterile filtration membranes.

Pharmaceutical composition and/or pharmaceutical formulations provided herein are useful as a medicament, e.g., for treating a neurodegenerative disorder, such as Alzheimer's disease.

### IV. Therapeutic and Prophylactic Uses

As disclosed herein, anti-CD300LB antibodies of the present disclosure may be used for preventing, reducing risk, or treating diseases and disorders. In some aspects, the present disclosure provides methods for preventing, reducing risk, or treating a neurodegenerative disease or disorder in an individual, such as, for example, Alzheimer's disease, comprising administering to the individual a therapeutically effective amount of an anti-CD300LB antibody of the present disclosure.

In some aspects, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some aspects, the subject or individual is a human.

An antibody provided herein (and any additional therapeutic agent) can be administered by any suitable means.

### V. Diagnostic Uses

In some aspects of any of the antibodies, any of the anti-CD300LB antibodies provided herein is useful for detecting the presence of CD300LB in a sample or an individual. The term “detecting” as used herein encompasses quantitative or qualitative detection. Provided herein are methods of using the antibodies of this disclosure for diagnostic purposes, such as the detection of CD300LB in an individual or in tissue samples derived from an individual. In some aspects, the individual is a human. In some aspects, the tissue sample is phagocytic cells (e.g., macrophages, dendritic cells), tumor tissue, cancer cells, etc.

The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain aspects, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

### VI. Articles of Manufacture

Provided herein are articles of manufacture (e.g., kit) comprising an anti-CD300LB antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some aspects, the kits may further comprise a second agent. In some aspects, the second agent is a pharmaceutically-acceptable buffer or diluting agent.

In some aspects of any of the articles of manufacture, the article of manufactures further comprises instructions for use in accordance with the methods of this disclosure. The instructions generally include information as to dosage, dosing schedule, and route of administration for the intended treatment. In some aspects, these instructions comprise a description of administration of the isolated antibody of the present disclosure (e.g., an anti-CD300LB antibody described herein) to prevent, reduce risk, or treat an individual having a disease, disorder, or injury, such as for example, Alzheimer's disease, according to any methods of this disclosure.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Construction of CD300LB Expression Plasmids for DNA Immunization and Protein Production

Several immunization approaches were used for developing antibodies directed against CD300LB. For DNA immunization, cDNA sequences encoding human CD300LB (SEQ ID NO: 127) and mouse CD300LB (SEQ ID NO:128) were cloned into the pCAGGS expression vector (KeraFAST EH1017) for DNA immunization. Expression of each CD300LB polypeptide was confirmed by transient transfection of the expression constructs into HEK293T cells, followed by extracellular flow cytometry using commercially available anti-CD300LB antibodies (Goat polyclonal antibodies, R&D, cat #AF2580 & AF2879; Kerafast Cat #EUC003). The expression constructs were then used for DNA immunization in mice as described below.

For protein immunization, nucleic acids encoding human or mouse CD300LB extracellular domain (SEQ ID NOs:130 and 131, respectively) were inserted into pD2610-v6 expression vector with a 3′ his-tag and Avi-tag or a 3′ mouse-Fc tag. The resulting clones were expressed in Expi293 cells and purified by Ni-NTA agarose (QIAGEN cat #30230) using the manufacturer's protocol. The CD300LB-Fc fusion polypeptide was used for immunization (as described below) and the CD300LB-His-Avi polypeptide was used for subsequent screening by ELISA binding.

For phage panning, the human CD300LB-Avi-His polypeptide was biotinylated at the AviTag by E. coli biotin ligase (BirA) according to the manufacturer's protocol.

### Example 2: Generation of Anti-CD300LB Hybridoma Antibodies

In order to obtain antibodies against CD300LB, the following procedures were used to generate hybridomas. Balb/c mice or Swiss mice (Charles River Laboratories, Wilmington, MA or Genovac, Freiburg, Germany) were co-immunized weekly or biweekly by tail vein injections or intradermal injections followed by electroporation with plasmid DNA expression constructs encoding full-length human and/or mouse CD300LB with or without adjuvant diluted in lactated Ringer's solution or phosphate buffered saline. Alternatively, Balb/c mice (Charles River Laboratories, Wilmington, MA) or CD300LB knockout mice (Taconic Biosciences, Rennselaer, NY) were co-immunized twice a week by subcutaneous or intraperitoneal injections of purified extracellular domain polypeptides of human CD300LB and mouse CD300LB (obtained as described above) with or without adjuvant. The animals received a total of 6-8 injections of either CD300LB expression plasmids or polypeptides. Sera from the animals were analyzed for reactivity to CD300LB by FACS on Baf/3 or Jurkat cells overexpressing human or mouse CD300LB (as described below). Three days following the final injection, the spleens and lymph nodes were harvested from the animals with CD300LB-specific titers for hybridoma cell line generation.

Lymphocytes from the spleens and lymph nodes of the immunized mice were isolated and then fused with SP2/mIL-6 (CRL-2016, American Type Culture Collection, Rockville, MD) or X63-Ag8 (Genovac, Freiburg, Germany) mouse myeloma cells via electrofusion (Hybrimmune, BTX, Holliston, MA) and incubated at 37° C., 5% CO₂, overnight in Clonacell-HY Medium C (STEMCELL Technologies, Vancouver, BC, Canada, Cat #03803). The following day, the fused cells were centrifuged and resuspended in 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (STEMCELL Technologies, Cat #03804) containing HAT components. The cells were plated into Nunc OmniTrays (Thermo Fisher Scientific, Rochester, NY) and allowed to grow at 37° C., 5% CO₂ for seven days. Fluorescent colonies were then selected and transferred into 96-well plates containing Clonacell-HY Medium E (STEMCELL Technologies, Cat #03805) using a Clonepix 2 (Molecular Devices, Sunnyvale, CA). Alternatively, hybridoma cells were single cell sorted into 96-well plates using a FACSMelody cell sorter (BD Biosciences, San Jose, CA). After five to ten days, tissue culture supernatants from the hybridomas were screened by ELISA against full length human or FACS on CD300LB over-expressing cells (the generation of which is described below).

### Example 3: Generation of Anti-CD300LB Antibodies Using Phage Display

Anti-human CD300LB human antibodies were isolated from a phage display human naïve scFv antibody library (SuperHuman 2.0, Distributed Bio, South San Francisco CA) by a series of four rounds of bio-panning against biotinylated human CD300LB-Avi-His (as described above). For each round of panning, decreasing concentrations of biotinylated CD300LB antigen was conjugated to streptavidin functionalized magnetic beads (Dynabeads M-280 Streptavidin). The CD300LB coated beads were washed twice times with PBST. 10¹³ phagemid (1 ml) were added to the non-conjugated magnetic beads and incubated at room temperature for 30 minutes twice to remove non-specific bead binders, and then the remaining phage were incubated with the CD300LB coated beads for 30 minutes (the incubation time was decreased for later panning rounds). After binding, the beads were washed three times with PBST and 3 times with PBS. Bound phage were eluted with TEA and used to infect freshly grown ER2738 cells. Phage libraries for subsequent rounds of panning were produced by co-infection of infected ER2738 cells with helper phage (M13K07). After the fourth round of panning, the infected ER2738 cells were diluted to produce single colonies, grown as single colonies, and scaled up for bacterial periplasmic extract (PPE) production. PPE (containing a scFv-V5 tag) was screened against human CD300LB protein by ELISA, and against the Jurkat cell line overexpressing CD300LB by FACS (as described below). Clones positive against the CD300LB protein and overexpressing cell line were sequenced, reformatted as human IgG1, and carried forward for further evaluation. Those that bound CD300LB at 10 μg/mL were reformatted to mouse IgG1 backbone for EC50 determination by cell binding and agonism activity analysis (as described below).

### Example 4: Generation of Human CD300LB and Murine CD300LB Expressing Cell Lines

Several cell lines were generated for screening the binding and agonism activity of CD300LB antibodies obtained as described herein. Baf/3 cell lines expressing murine CD300LB (muCD300LB) were a gift by Dr. Jiro Kitaura (Juntendo University, Japan). Briefly, Baf/3 cell lines were generated by retroviral transduction with pMXs FLAG-tagged DAP12 (puromycin selection), followed by pMXs-Myc-tagged muCD300LB (blasticidin selection). Cells were engineered to express a luciferase reporter gene under the control of an NFAT (nuclear factor of activated T-cells) promoter. The system Cignal Lenti NFAT-Luciferase virus (Qiagen) was adopted. Jurkat-Lucia™ NFAT reporter cell lines were purchased from Invivogen. The cells were derived from the human T Lymphocyte-based Jurkat cell line by stable integration of an NFAT-inducible Lucia reporter construct. pLenti-EF1a constructs expressing huCD300LB-DAP12 and muCD300LB-DAP12 fusion proteins (SEQ ID NOs: 133 and 134, respectively) were used to generate Jurkat-Lucia™ NFAT cells stably expressing huCD300LB fused to DAP12 or muCD300LB fused to DAP12. Alternatively, Jurkat-Lucia™ NFAT were first infected with a lentiviral construct expressing human DAP12-GFP (Genecopoeia) and later infected with p-Lenti-EF1a construct expressing huCD300LB or muCD300LB. 293 free-style constitutively expressing huCD300A (SEQ ID NO: 135), huCD300LB-DAP12 fusion (SEQ ID NO: 133), huCD300C-DAP12 fusion (SEQ ID NO: 136), huCD300LD-DAP12 fusion (SEQ ID NO: 137), huCD300E-DAP12 fusion (SEQ ID NO: 138), huCD300F (SEQ ID NO: 139), and huCD300G (SEQ ID NO: 140) were generated by lentiviral infection. Myc-tag was inserted in each construct following the signal peptide. For murine CD300A (SEQ ID NO: 141), murine CD300LD (SEQ ID NO: 142), and murine CD300F (SEQ ID NO: 143) Jurkat-Lucia™ NFAT stable cell line generation, lentiviral constructs were purchased from Genecopoeia. Lenti #CS-Mm14660-Lv105-01 was used for muCD300A, lenti #CS-Mm14662-Lv105-01 for CD300LD, and lenti #CS-Mm23594-Lv105-01 for CD300F.

### Example 5: Binding of Anti-CD300LB Antibodies to Jurkat Cells Expressing Recombinant Human CD300LB and to BaF/3 Cells Expressing Recombinant Murine CD300LB

Anti-CD300LB hybridoma supernatants or phage PPE were screened initially on their ability to bind either Jurkat cells overexpressing recombinant human CD300LB (huCD300LB) or BaF/3 cells overexpressing recombinant murine CD300LB (muCD300LB). Binding was assessed by flow cytometry. Overexpressing cells were harvested, washed, and labeled with Aqua Live/Dead or propidium iodide for viability discrimination. Cells (1×10⁵ per well) were aliquoted into 96 well plates and incubated with 100 μL supernatant for 1 hour before washing twice with 100 μL ice cold FACS buffer (PBS+2% FBS). Cells were then incubated with APC-conjugated anti-mouse IgG or Alexa Fluor 647-conjugated anti-V5 tag secondary antibodies for 20 minutes in the dark on ice. Cells were again washed twice with ice-cold FACS buffer and resuspended in a final volume of 200 μL of FACS buffer. Flow cytometry analysis was then performed using the FACSCanto system (BD Biosciences). Binding data was expressed as Geometric Mean Fluorescence Intensity (gMFI); those with a gMFI above that of isotype control were selected. Approximately 1260 hybridoma supernatants and 576 phage PPEs were screened for binding in this way.

Purified anti-CD300LB antibodies of the present disclosure were evaluated for their binding affinity to either Jurkat cells overexpressing recombinant huCD300LB or BaF/3 cells overexpressing recombinant muCD300LB. Binding of anti-CD300LB antibodies to the cells was determined as follows. Cells were harvested, washed, and labeled with propidium iodide for viability discrimination. After washing the cells with PBS, 1×10⁵ cells were aliquoted per well in 96-well U-bottom plates and incubated with 100 μL of purified anti-CD300LB antibody at various concentrations in FACS buffer (PBS+2% FBS). After this primary incubation, the supernatant was removed via centrifugation, the cells washed twice with 150 μL of ice-cold FACS buffer, and then incubated with the appropriate secondary antibody on ice for 30 minutes. Following the secondary antibody incubation, the cells were again washed twice with ice-cold FACS buffer and resuspended in a final volume of 200 L FACS buffer. Flow cytometry analysis was then performed using the FACSCanto system (BD Biosciences).

To determine affinity, antibodies at increasing concentrations were used to stain Jurkat cells overexpressing huCD300LB and geometric MFI values measured by the flow cytometer were analyzed on Prism (Graphpad) software. To determine EC50s, the Drake & Klakamp equation was used (Drake & Klakamp, Journal of Immunological Methods 318 (2007) 147-152):

$$Y = ( ( F max - B ) / ( n * M ) ) * [ [ ( K + X + n * M ) - S Q R T \{ ( {( K + X + n * M )}^{∧} 2 ) - 4 * ( n * M * X ) \} ] / 2 ] + B$$

In these analyses, the following values were constrained according to experimental conditions: M_{c}=concentration of cells (nM, 1.66030217499585E-06). The results of these binding studies are shown in Error! Reference source not found.

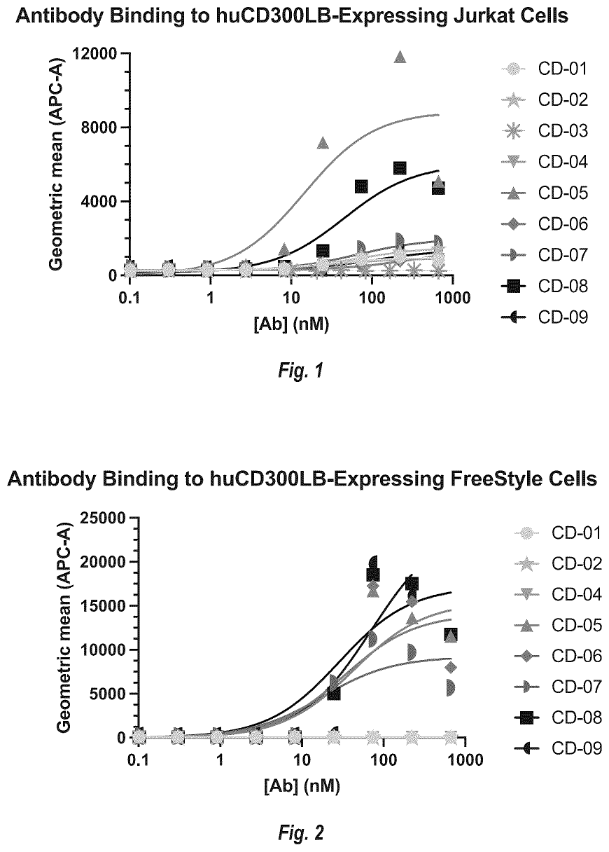

As shown in FIG. 1, anti-CD300LB antibodies of the present disclosure bound to huCD300LB-expressing Jurkat cells to varying degrees in a dose-dependent manner. These results indicated that anti-CD300LB antibodies of the present disclosure bind to human CD300LB expressed in cells.

The apparent affinity of certain anti-CD300LB antibodies of the present disclosure as measured by the cell binding assay described above is shown below in Table 1.

TABLE 1

| Antibody | Apparent KD (nM) |
|---|---|
| CD-01 | 27 |
| CD-02 | 64 |
| CD-03 | >1000 |
| CD-04 | 32 |
| CD-05 | 15 |
| CD-06 | 329 |
| CD-07 | 66 |
| CD-08 | 45 |
| CD-09 | 109 |

These results showed that anti-CD300LB antibodies of the present disclosure bind to human CD300LB with affinities in the range of 15-329 nM.

The geometric mean fluorescence intensity (gMFI) of the Baf/3 cells stained with anti-CD300LB antibodies recognizing muCD300LB are shown in Table 2 below.

TABLE 2

| | BaF/3 overexpressing | BaF/3 parental |
|---|---|---|
| Antibody | muCD300LB (gMFI) | cells (gMFI) |
| CD-10 | 6258 | 209 |
| CD-11 | 10907 | 209 |
| CD-12 | 9798 | 215 |
| CD-13 | 22114 | 210 |
| CD-14 | 19436 | 219 |
| CD-15 | 8334 | 215 |
| CD-16 | 8966 | 212 |

These results showed that anti-CD300LB antibodies of the present disclosure are capable of binding to murine CD300LB.

### Example 6: Crossreactivity of Anti-CD300LB Antibodies to Other Members of the Human CD300 Family

Other members of the human CD300 family share sequence homology with human CD300LB. Thus, counter screens against binding of the anti-CD300LB antibodies to huCD300A, huCD300C, huCD300LD, huCD300E, huCD300LF, and huCD300G by stable overexpression in Freestyle HEK cells (generation of which is described above) were performed as follows. Cells were counted and labeled with 200 nM CFSE, 500 nM VioBlue, or a combination of the two to discriminate between CD300LB, Parental cells, and the other CD300 family members. Cells were washed thoroughly with PBS containing 2% fetal bovine serum then resuspended at a total of 240,000 cells per well. Hybridoma supernatants (100 L) or purified antibodies at various concentrations were added and incubated for one hour before the cells were washed twice again and stained with an APC-labeled anti-human or anti-mouse secondary antibody. Cells were then analyzed using a BD FACSCanto II. Anti-CD300LB antibodies were analyzed for specific binding to human CD300LB by FlowJo software by gating on live cells, then the APC geometric mean fluorescence intensity (gMFI) calculated for each labeled population. The results are shown in Error!Reference source not found. and Error! Reference source not found.

As shown in FIG. 2, anti-CD300LB antibodies of the present disclosure were capable of binding to huCD300LB recombinantly expressed in Freestyle HEK cells in a dose-dependent manner.

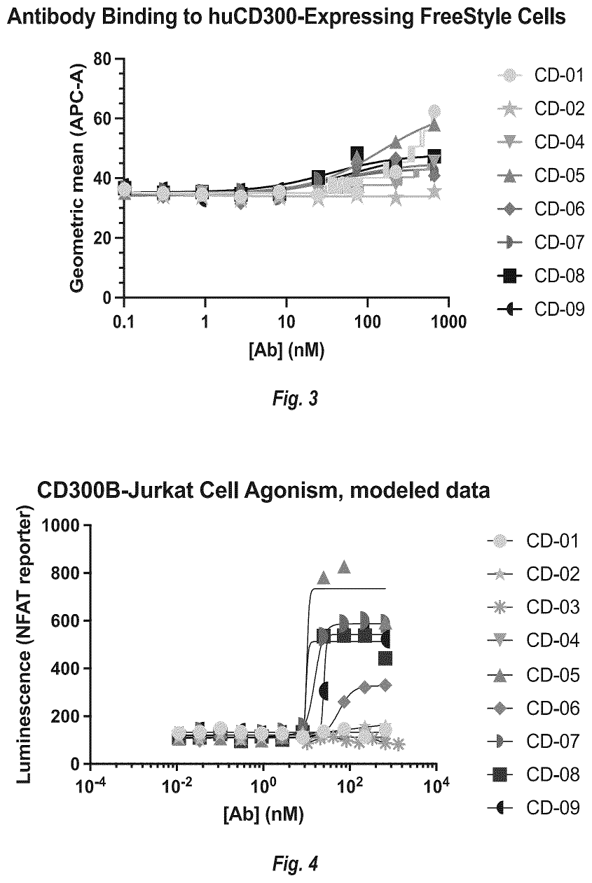

As shown in FIG. 3, anti-CD300LB antibodies of the present disclosure exhibited minimal binding to non-transfected Freestyle HEK cells.

Freestyle HEK cells were transfected with huCD300A, huCD300C, huCD300D, huCD300E, and huCD300G separately. Then, five stable cell lines from these transfections were pooled; the combined cells are herein known as CD300A/C/D/E/G. Freestyle HEK cells were also transfected with huCD300LB and huCD300F. All transfected cells were then tested for binding of anti-CD300LB antibodies of the present disclosure. Table 3 (values presented as gMFI) below shows binding of anti-CD300LB antibodies of the present disclosure to other human CD300 family members. As shown in Table 3, anti-CD300LB antibodies of the present disclosure do not bind to other human CD300 family members.

TABLE 3

| Antibody | CD300A/C/D/E/G | CD300LB | CD300F | Parental |
|---|---|---|---|---|
| CD-01 | −21.5 | 45 | −26 | −42.5 |
| CD-02 | −6 | 107.5 | −93 | 44 |
| CD-03 | 1911 | 162725 | 2090 | 861 |
| CD-04 | 52.5 | 189.5 | 163 | −16 |
| CD-05 | 2512.5 | 113028.5 | 2731.5 | 691.5 |
| CD-06 | 778.75 | 80928.25 | 885.5 | 290 |
| CD-07 | 42.5 | 31549 | 112 | 73.5 |
| CD-08 | 166.5 | 56822 | 212 | 71 |
| CD-09 | −25.5 | 37902 | 28 | 68.5 |

### Example 7: Crossreactivity of Anti-CD300LB Antibodies to Members of the Murine CD300 Family

Purified anti-CD300LB antibodies of the present disclosure were analyzed for cross-reactivity to other murine CD300 family members stably expressed in Jurkat cells (generation of which is described above). Cells were counted and labeled with 200 nM CFSE, 500 nM VioBlue, or a combination of the two to discriminate between muCD300LB, Parental cells, CD300F, and the other CD300 family members. Cells were washed thoroughly with PBS containing 2% fetal bovine serum then resuspended at a total of 240,000 cells per well. Purified antibodies at 10 μg/mL were added and incubated for one hour before the cells were washed twice again and stained with an APC-labeled anti-mouse secondary. Cells were then analyzed using a BD FACSCanto II. Antibodies were analyzed for specific binding to muCD300LB by FlowJo software by gating on live cells, then the APC gMFI calculated for each labeled population by subtracting the gMFI of the isotype control. The gMFI results of the analysis are presented below in Table 4.

TABLE 4

| | muCD300D | muCD300LB | muCD300F | Parental |
|---|---|---|---|---|
| Antibody | gMFI | gMFI | gMFI | gMFI |
| CD-10 | −1.35 | 1610.2 | −0.8 | −0.4 |
| CD-11 | 1.05 | 4663.2 | 2.35 | 3.6 |
| CD-12 | −3.6E−15 | 4209.7 | 2.1 | 1.55 |
| CD-13 | 10.5 | 7601.2 | 13.55 | 11.8 |
| CD-14 | 13.3 | 9546.2 | 18.35 | 13.7 |
| CD-15 | 2.05 | 4144.7 | 1.85 | 3.3 |
| CD-16 | 0.15 | 4567.2 | −0.35 | 5.7 |
| CD-17 | 0.8 | 7916.7 | 1.7 | 4.9 |
| CD-18 | 2.8 | 2521.7 | 6.6 | 2.6 |

As shown in Table 4, anti-CD300LB antibodies of the present disclosure that were tested in these studies bound to murine CD300LB. Certain anti-CD300LB antibodies of the present disclosure exhibited minimal binding (above background) to murine CD300F (anti-CD300LB antibodies CD-13, CD-14, and CD-18). Certain anti-CD300LB antibodies were capable of binding to a low degree to murine CD300D (anti-CD300LB antibodies CD-13 and CD-14). Taken together, these results showed that certain anti-CD300LB antibodies of the present disclosure exhibited some degree of binding to multiple murine CD300 family members, including muCD300LB, muCD300D, and muCD300F.

### Example 8: Species Crossreactivity of Anti-CD300LB Antibodies

Purified anti-CD300LB antibodies of the present disclosure were also evaluated for species cross-reactivity to mouse and human CD300LB stably expressed in Jurkat cells (generation of which is described above). Cells were counted and labeled with 200 nM CFSE, 500 nM VioBlue, or a combination of the two to discriminate between human CD300LB, Parental cells and mouse CD300LB. Cells were washed thoroughly with PBS containing 2% fetal bovine serum then resuspended at a total of 240,000 cells per well. Hybridoma supernatants (100 L) or purified antibodies at 10 μg/mL were added and incubated for one hour before the cells were washed again and stained with an APC-labeled anti-human or anti-mouse secondary. Cells were then analyzed using a BD FACSCanto II. Antibodies were analyzed for binding to mouse or human CD300LB by FlowJo software by gating on live cells, then the APC gMFI calculated for each labeled population by subtraction of the gMFI of the appropriate isotype control. The results of these analyses are shown below in Table 5.

TABLE 5

| | Parental | huCD300LB | muCD300LB |
|---|---|---|---|
| Antibody | gMFI | gMFI | gMFI |
| CD-01 | 189 | 309 | 860 |
| CD-02 | −131 | 1132 | 23 |
| CD-04 | 7 | 193 | 18 |
| CD-05 | 104 | 1806 | 172 |
| CD-06 | −73.2 | 3183 | −48.7 |
| CD-10 | 20 | 18 | 284 |
| CD-11 | 34 | 50 | 320 |
| CD-13 | 238 | 324 | 1190 |
| CD-14 | 42 | 59 | 950 |
| CD-15 | 2 | 22 | 506 |
| CD-16 | 15 | 31 | 504 |
| CD-17 | 25 | 13 | 1685 |
| CD-18 | 105 | 114 | 643 |

As shown in Table 5, certain anti-CD300LB antibodies of the present disclosure were capable of binding to both human CD300LB and murine CD300LB (anti-CD300LB antibodies CD-01, CD-13, CD-14, and CD-18), based on this assay. Certain anti-CD300LB antibodies of the present disclosure effectively bind to human CD300LB but did not display binding to murine CD300LB (CD-02, CD-04, CD-06).

In addition, supernatants or purified antibodies were tested for species crossreactivity by comparing the binding of human, mouse, and cynomolgous monkey CD300LB expressing cell lines. HEK293 were transfected with 13.5 g of human, mouse, and cyno CD300LB expression constructs using the Mirus system in 10 cm tissue culture dishes. Cells were then incubated for 24 hours in complete medium. Cells were harvested by incubation in TrypLE Express, then washed with PBS and labeled with 200 nM CFSE, 500 nM VioBlue, or a combination of the two to discriminate between human, mouse, and cyno CD300LB expressing cells. Cells were washed thoroughly with PBS containing 2% fetal bovine serum then resuspended at a total of 240,000 cells per well. Purified antibodies at 10 μg/mL were added and incubated for one hour before the cells were washed again and stained with an APC-labeled anti-human or anti-mouse secondary. Cells were then analyzed using a BD FACSCanto II. Antibodies were analyzed for specific binding to human, mouse, or cyno CD300LB by FlowJo software by gating on live cells, then the APC gMFI calculated for each labeled population minus the gMFI of the isotype control.

The results of these studies are shown below in Table 6.

TABLE 6

| | huCD300LB HEK293 | cynoCD300LB HEK293 | Parental |
|---|---|---|---|
| | gMFI | gMFI | HEK293 |
| CD-01 | 166.2 | 190.15 | 10.3 |
| CD-02 | 578.2 | 13.5 | 20.15 |
| CD-03 | 38.7 | 17.15 | 3.8 |
| CD-04 | 207.2 | 72.15 | 26.5 |
| CD-05 | 933 | −62.5 | −67.5 |
| CD-06 | 314.3 | −51 | −70 |
| CD-07 | 685.5 | 39 | 110.5 |
| CD-08 | 774 | −43.5 | −49.5 |
| CD-09 | 445 | −19.5 | 7.5 |

As shown in Table 6, certain anti-CD300LB antibodies of the present disclosure were capable of binding to human CD300LB and cyno CD300LB (anti-CD300LB antibodies CD-01, CD-03, and CD-04).

### Example 9: Analysis of Anti-CD300LB Antibody Crossreactivity to CD300 Family Members by Surface Plasmon Resonance

In addition to the screening of antibodies for cross-reactivity by FACS analysis, surface plasmon resonance (SPR) analysis was used to determine the binding of antibodies to recombinant huCD300 and muCD300 family members (orthologs) conjugated to an Fc (human CD300A-Fc SEQ ID NO:144; human CD300LB-Fc SEQ ID NO:145; human CD300C-Fc SEQ ID NO: 146; human CD300D-Fc SEQ ID NO: 147; human CD300E-Fc SEQ ID NO: 148; human CD300F-Fc SEQ ID NO: 149; murine CD300A-Fc SEQ ID NO: 150; murine CD300LB-Fc SEQ ID NO:151; murine CD300C-Fc SEQ ID NO: 152; murine CD300D-Fc SEQ ID NO: 153, and murine CD300E-Fc SEQ ID NO: 154).

Binding of mouse anti-CD300LB IgG1 antibodies of the present disclosure to human and murine CD300 orthologs were evaluated by surface plasmon resonance (SPR) using a Carterra LSA instrument (Carterra, Salt Lake City, UT). Briefly, anti-CD300LB antibodies were prepared by diluting to 10 μg/ml [MR1] in 10 mM Acetate, pH 4.25 (Carterra), at 200 L/well. A HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μL of each mixed in vial immediately before running assay). After switching to the multi-channel array flow cell, the antibodies were injected over the activated chip in a 96-spot array for 10 minutes. The remaining unconjugated active groups on the chip were then blocked by injecting 1M Ethanolamine pH 8.5 (Carterra) for 7 minutes using the single channel flow cell. After priming with running buffer (HBS-TE, Carterra) with 0.5 mg/ml BSA (Sigma), the immobilized anti-CD300LB antibodies were tested for their ability to bind human and mouse recombinant CD300LB extracellular domains. Human CD300 family members and mouse CD300A were hIgG1-Fc fusions, while mouse CD300LB, mouse CD300C, mouse CD300D, and mouse CD300E were mouse IgG2a-Fc fusions (R&D Systems). Binding was assessed by injecting each analyte over the entire antibody array using the single channel flow cell. CD300 analytes were diluted to 300 nM or 600 nM in running buffer and injected in series without regeneration. Two or three buffer blanks were run between each mouse or human ortholog, respectively. Data were processed and analyzed using NextGenKIT high-throughput kinetics analysis software (Carterra).

Results of anti-CD300LB antibody binding to human CD300 family members is provided below in Table 7. Control antibody binding values have been subtracted from the binding data presented in Table 7.

TABLE 7

| | Human | Murine | Human | Human | Human | Human | Human |
|---|---|---|---|---|---|---|---|
| | CD300LB- | CD300LB- | CD300A- | CD300C- | CD300D- | CD300E- | CD300F- |
| | Fc | Fc | Fc | Fc | Fc | Fc | Fc |
| CD-01 | 3.71 | 29.045 | 0.175 | 0.805 | 0.575 | −0.105 | 0.075 |
| CD-02 | 21 | 5.555 | 0.735 | 0.425 | 0.76 | 0.62 | 0.87 |
| CD-03 | 43.045 | 7.555 | 0.095 | 0.165 | 0.4 | 0.18 | 0.42 |
| CD-05 | 51.045 | 3.655 | 0.24 | 0.06 | 0.115 | 0.195 | 0.045 |

As shown in Table 7, anti-CD300LB antibodies of the present disclosure were specific in binding to human CD300LB; anti-CD300LB antibodies of the present disclosure did not display binding to other human CD300 family members.

Results of anti-CD300LB antibody binding to murine CD300 family members is provided below in Table 8. Control antibody binding values have been subtracted from the binding data presented in Table 8.

TABLE 8

| | mCD300A | mCD300LB | mCD300C | mCD300D | mCD300E |
|---|---|---|---|---|---|
| CD-10 | −2.5 | 370.5 | 11.8 | −0.6 | 11.3 |
| CD-11 | −2.1 | 87.7 | 37.8 | −0.1 | 7.4 |
| CD-12 | 4.2 | 43.2 | 2.5 | 0.6 | −0.6 |
| CD-13 | 4.4 | 82.4 | 43.6 | 1.1 | 4.5 |
| CD-14 | 2.7 | 29.0 | 17.9 | 1.5 | 3.6 |
| CD-15 | 7.1 | 50.0 | 13.3 | 1.7 | 0.8 |
| CD-16 | −1.9 | 310.4 | 34.5 | −0.1 | 8.6 |
| CD-17 | 2.9 | 11.3 | 1.5 | 0.8 | −0.7 |
| CD-18 | 2.9 | 328.5 | 1.5 | 0.6 | −0.1 |

As shown in Table 8, anti-CD300LB antibodies of the present disclosure were capable of binding to murine CD300LB but did not display binding to other murine CD300 family members. Anti-CD300LB antibodies CD-13, CD-14, CD-15, and CD-16 showed binding to mCD300C.

### Example 10: Epitope Binning Analysis of Anti-CD300LB Antibodies

Epitope binning analysis was performed on the anti-CD300LB antibodies of the present disclosure by performing a tandem injection approach using a Carterra LSA instrument (Carterra, Salt Lake City, UT). Briefly, purified anti-CD300LB antibodies of the present disclosure (anti-CD300LB antibodies CD-01, CD-02, CD-03, CD-04, CD-05, CD-06, CD-07, CD-08, CD-09) and anti-his IgG were diluted to 20 μg/ml in 10 mM Acetate, pH 4.25 (Carterra), at 200 μl/well. A HC200M sensor chip (Carterra) was activated using the single channel flow cell with a 7-minute injection of a 1:1:1 mixture of 100 mM MES pH 5.5, 100 mM sulfo-NHS, 400 mM EDC (as described above). After switching to the multi-channel array flow cell, the 20 μg/ml dilutions of antibodies were injected over the activated chip in a 96-spot array for 15 minutes. The remaining unconjugated active groups on the chip were then blocked by injecting 1M Ethanolamine pH 8.5 (Carterra) over the entire chip surface for 7 minutes using the single channel flow cell.

After priming with running buffer (HBS-TE, Carterra) containing 0.5 mg/ml BSA (Sigma), the immobilized antibodies were tested for their ability to bind to His-tagged recombinant human CD300 extracellular domain. Bound CD300 was removed by two one-minute injections of Glycine pH 2.0 (Carterra). For each cycle, CD300 was injected over the chip at 100 μg/ml, followed by a test antibody diluted to 200 μg/ml in running buffer. Data were processed and analyzed using Epitope high-throughput binning analysis software (Carterra). Antibodies which were able to bind antigen captured by an immobilized antibody were designated as “sandwich” or “pairing” antibodies, and these antibodies were assigned into a different epitope bin from that of the immobilized antibody. A matrix of pairing and non-pairing antibodies was constructed from the binding results of these experiments, which allowed for an epitope bin landscape of the anti-CD300LB antibodies to be generated. The results from these studies are provided below in Table 9.

TABLE 9

| Bin | Antibody |
|---|---|
| 1a | CD-01 |
| 1b | CD-02 |
| 1c | CD-05, CD-06, CD-08, CD-09 |
| 1d | CD-07 |
| 1e | CD-04 |
| 2 | CD-03 |

As shown in Table 9, anti-CD300LB antibodies of the present disclosure displayed a variety of binning profiles, characterized by bin 1a, bin 1b, bin 1c, bin 1d, bin 1e, and bin 2. Bin 1 anti-CD300LB antibodies of the present disclosure (bin 1a, bin 1b, bin 1c, bin 1d, and bin 1e) are capable of at least partially blocking binding of other bin 1 anti-CD300LB antibodies to human CD300LB.

### Example 11: Agonistic Activity of Anti-CD300LB Antibodies in Jurkat Cell Overexpressing Recombinant Human CD300B

Human CD300LB-NFAT-Lucia Jurkat reporter cells (with or without chimerization to human DAP12 were used to screen hybridoma supernatants and purified antibodies for their ability to activate huCD300LB receptor signaling. Antibodies bound to a solid surface act to cluster the CD300LB receptor, activating an NFAT-lucia luciferase system to induce the production of a secreted enzyme that converts a non-luminescent substrate into a luminescent one, in proportion to receptor activation by the antibodies.

Hybridoma supernatants were initially screened for cell binding by FACS (described above) and agonism before selection for purification. Non-tissue culture treated 96-well flat bottom plates were coated with 5 g/mL capture antibody (goat anti-mouse IgG, Jackson Immunoresearch, catalogue AB_2338468) for 5 hours, then washed with PBS and blocked with 2% BSA overnight. Wells were washed extensively with PBS then incubated with 100 μL undiluted hybridoma supernatant. After further washing, 100,000 huCD300LB-overexpressing jurkat cells were added to each well and incubated for 24 hours. Secreted Lucia was then measured by pipetting 20 μL of cell culture supernatant into an opaque-walled 96 well plate and adding 50 μL of QUANTI-Luc plus reagent (Invivogen, catalog rep-qlc2). Luminescence was measured immediately after using a BioTek plate reader. Supernatants were selected as positive in this assay if they induced luminescence above background wells, calculated based on the luminescence reading of isotype controls and irrelevant supernatants generated from hybridoma, around 100. This assay was used to screen ˜1400 supernatants.

Purified antibodies were incubated on non-tissue culture-treated 96 well flat bottom plates overnight at various concentrations. The plates were then washed thoroughly with PBS (100 μL, rinsed three times) before CD300LB over-expressing Jurkat cells were counted, washed in PBS, and plated at 100,000 cells on top of the antibody lawn. Following a 24-hour incubation, secreted Lucia was measured by pipetting 20 μL of cell culture supernatant into an opaque-walled 96 well plate and adding 50 μL of QUANTI-Luc plus reagent (Invivogen, catalog rep-qlc2). Luminescence was measured immediately after using a BioTek plate reader. To determine affinity, a log(agonist) vs. response Variable slope (four parameters), least squares fit model was used. The results of these binding studies are shown in FIG. 4.

As shown in FIG. 4, anti-CD300LB antibodies of the present disclosure were capable of activating CD300LB, as evidenced by an increase in luminescence associated with the NFAT reporter assay used in these studies. These results indicated that soluble anti-CD300LB antibodies of the present disclosure activate CD300LB and increase CD300LB signaling.

### Example 12: Agonistic Activity of Plate-Bound Anti-CD300LB Antibodies and Mouse BaF/3 Cell Line

Supernatants from hybridoma were screened using plate-bound agonism in muCD300LB overexpressing BaF/3 cells. Hybridoma supernatants were initially screened for agonism before selection for purification. Non-tissue culture treated 96-well flat bottom plates were coated with 5 g/mL capture antibody (goat anti-mouse IgG, Jackson Immunoresearch, catalogue AB_2338468) for 5 hours, then washed with PBS and blocked with 2% BSA overnight. Wells were washed extensively with PBS then incubated with 100 μL undiluted hybridoma supernatant. After further washing, 100,000 muCD300LB-overexpressing BaF/3 cells were added to each well and incubated for 6 hours. For 96-well plates, 100 μL of ONE-Glo™ Reagent (Promega, E6120) was added to the cells grown in 100 μL of medium. After a 3-minute incubation luminescence was measured using a BioTek plate reader. Supernatants were selected as positive in this assay if they induced luminescence above background wells, calculated based on the luminescence reading of isotype controls and irrelevant supernatants generated from hybridoma. The isotype control value in these experiments was around 700. The results from these studies are shown below in Table 10.

TABLE 10

| Supernatant | Luminescence in BaF/3 |
|---|---|
| CD-11 | 22434 |
| CD-12 | 22762 |
| CD-13 | 22399 |
| CD-14 | 19657 |
| CD-15 | 22966 |
| CD-16 | 792 |
| CD-17 | 24736 |
| CD-18 | 12986 |

As shown in Table 10, anti-CD300LB antibodies of the present disclosure were capable of activating CD300LB when plate-bound, as evidenced by an increase in luminescence in the assay. These results indicated that anti-CD300LB antibodies of the present disclosure activate CD300LB and increase CD300LB signaling.

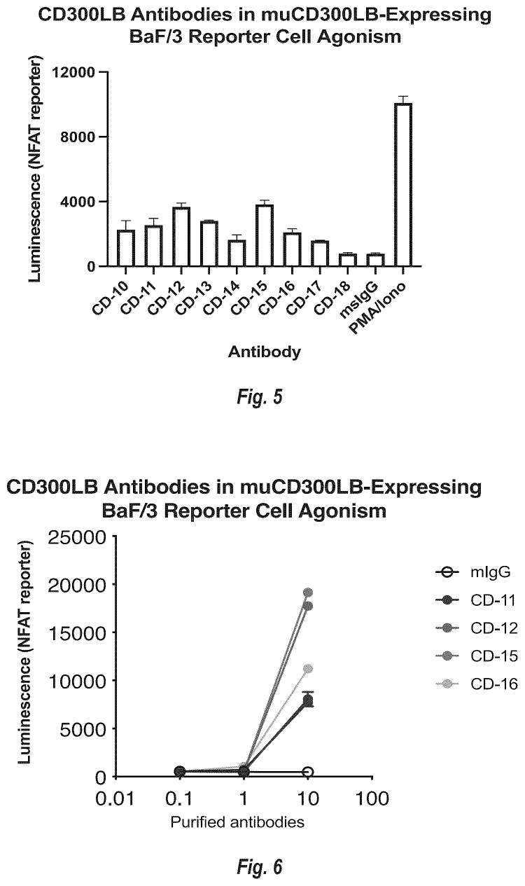

In additional studies, purified antibodies were incubated on non-tissue culture-treated 96 well flat bottom plates overnight at a concentration of 10 μg/mL for screening purposes, and varying concentrations after that. The plates were then washed thoroughly with PBS (100 μL, rinsed three times) before muCD300LB-overexpressing BaF/3 cells were counted, washed in PBS, and plated at 100,000 cells on top of the plated antibody lawn. Following a 6-hour incubation, luminescence was measured using methods as described above. Anti-CD300LB antibodies were identified as positive in this assay if they induced luminescence above that observed in background wells, calculated based on the luminescence reading of isotype controls. PMA addition to the cells served as a positive control. The results of these studies are shown in FIG. 5 and FIG. 6.

As shown in FIG. 5 and FIG. 6, anti-CD300LB antibodies of the present disclosure were effective at activating CD300LB in this assay, as measured by an increase in luminescence. These results indicated that anti-CD300LB antibodies of the present disclosure activate CD300LB and increase CD300LB signaling.

### Example 13: Binding of Anti-CD300LB Antibodies to Primary Human Monocytes and Macrophages

Human monocytes from various donors were isolated from whole blood using RosetteSep Human Monocyte Enrichment Cocktail (Stemcell technologies) and Ficoll centrifugation per manufacturer protocols. After lysing red blood cells with ACK lysing buffer, monocytes were resuspended in complete media (RPMI, 10% FBS, Pen/Strep, L-glutamine, HEPES, non-essential amino acid, Sodium pyruvate). To obtain macrophages from these isolated monocytes, 100 ng/ml human M-CSF and 8% v/v human serum were added to the cells for 5-7 days. Cells were counted on the day of the experiment and plated at 100,000 cells per well in a round-bottom 96 well plate. Cells were incubated with TruStain Fc block and 10 μg/mL antibody for 1 hour on ice before washing with 250 μL ice cold PBS twice. Cells were then incubated with the appropriate APC-conjugated secondary antibodies for 20 minutes in the dark on ice. Finally, cells were washed twice with ice cold FACS buffer and then resuspended in 100 μL FACS buffer containing 1% propidium iodide. Samples were immediately analyzed using a BD FACSCanto flow cytometer. The gMFI was calculated using FlowJo software minus the gMFI of the appropriate isotype control. The results of these studies are provided in Table 11. Control antibody binding values have been subtracted from the binding data presented in Table 11.

TABLE 11

| | Monocyte | Monocyte | Monocyte | Monocyte | Macrophage | Macrophage | Macrophage |
|---|---|---|---|---|---|---|---|
| Antibody | donor 1281 | donor 1282 | donor 1283 | donor 1284 | donor 1275 | donor 1277 | donor 1278 |
| CD-05 | 736 | 1555 | 935 | 166 | 60965 | 68623 | 175359 |
| CD-06 | 1651 | 3437 | 2467 | 1014 | 26949 | 23995 | 169035 |
| CD-07 | 43 | 468 | 47 | −189 | 2705 | 3792 | 211406 |
| CD-08 | 2967 | 4467 | 1922 | 1404 | 3580 | 2797 | 135067 |
| CD-09 | 1369 | 3072 | 2584 | 1038 | 19672 | 15709 | 13296 |

As shown in Table 11, anti-CD300LB antibodies of the present disclosure are capable of binding to human monocytes and to human macrophages.

### Example 14: Determination Anti-CD300B Specific Binding to CD300LB Using Wild Type CD300LB Macrophages and CD300LB Knockout Macrophages

To further examine the specificity of the anti-CD300LB antibodies to muCD300LB, bone marrow macrophages (BMM) from wild type (WT) and CD300LB knockout mice (KO) were analyzed for antibody binding by flow cytometry. Bone marrow from these mice was harvested and plated into three 20 cm PETRI dishes in complete media containing 50 ng/mL murine M-CSF (Peprotech). Media was changed every three days until day 6, when cells were harvested. Cells were detached with PBS 3 mM EDTA, washed and counted and plated at 100,000 cells/well in a 96 well plate. Cells were incubated with murine Fc block (Clone 2.4G2, BD) then with 10 μg/mL anti-CD300LB antibodies for 30 minutes on ice. Cells were washed twice with 150 μL ice cold FACS buffer then stained with goat anti-mouse IgG conjugated to APC (goat anti mouse APC, Jackson Immunoresearch cat #115-136-071 lot 140337). Cells were washed twice more with 150 μL FACS buffer then resuspended to 100 μL in FACS buffer analyzed using a BD Canto II flow cytometer to calculate the geometric MFI minus the gMFI of the isotype control. Dead cells were stained with live/dead staining kit (Invitrogen). The results of these studies are shown in Table 12. —

TABLE 12

| Antibody | Wt BMM gMFI | KO BMM gMFI |
|---|---|---|
| CD-10 | 192 | 74 |
| CD-11 | 349 | −28 |
| CD-12 | 413 | −9 |
| CD-15 | 256 | 17 |
| CD-16 | 233 | −31 |
| CD-17 | 1234 | 460 |
| CD-18 | 1292 | 543 |

As shown in Table 12, anti-CD300LB antibodies of the present disclosure were capable of binding to murine CD300LB on mouse bone marrow macrophages.

### Example 15: Syk Phosphorylation by Anti-CD300B Antibodies in Primary Human Macrophages

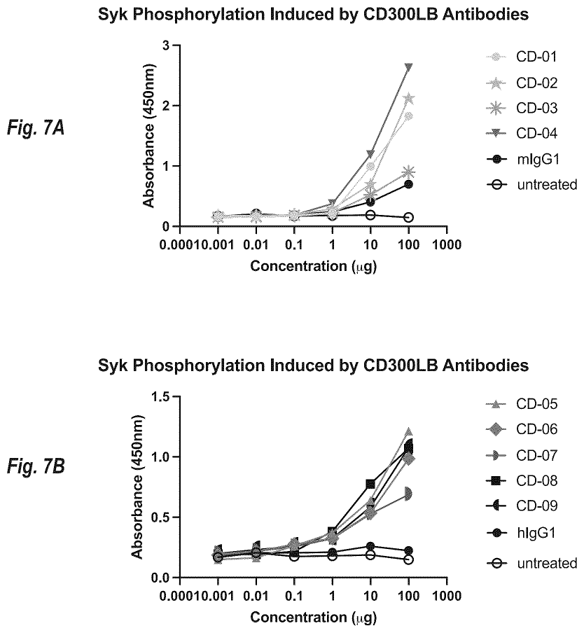

Primary human macrophages were used to determine the phosphorylation of Syk following incubation with anti-CD300LB antibodies of the present disclosure. To obtain macrophages from isolated monocytes (isolation described above), 100 ng/ml human M-CSF and 8% v/v human serum were added to the cells for 5-7 days. On day 7 cells were scraped, dissociated by pipetting, then plated at 100,000 cells/well on to a lawn of plate-bound anti-CD300LB antibodies that had been coating in PBS for four hours. Cells were incubated for 5, 30, and 60 minutes at 37° C. To harvest the cells, the plates were centrifuged at 1200×g for 5 minutes at 4° C. then transferred onto ice and lysed with 100 μL lysis buffer with inhibitors (N-PER plus inhibitors, Thermofisher, cat 87792) per well. After 5 minutes on ice cells were transferred to −80° C. pSyk was measured using PathScan® Phospho-Syk (Tyr525/526) Sandwich ELISA Kit #7970 following manufacturer's instructions. Absorbance was read at 450 nm with a BioTek plate reader. The results of this experiment are shown in FIG. 7A and FIG. 7B.

As shown in FIG. 7A and FIG. 7B, anti-CD300LB antibodies of the present disclosure were capable of increasing Syk phosphorylation in primary human macrophages. These results indicated that anti-CD300LB antibodies of the present disclosure are capable of activating CD300LB and increasing CD300LB signaling in cells, as evidenced by an increase in Syk phosphorylation in this assay.

### Example 16: DAP12 Phosphorylation by Anti-CD300LB Antibodies

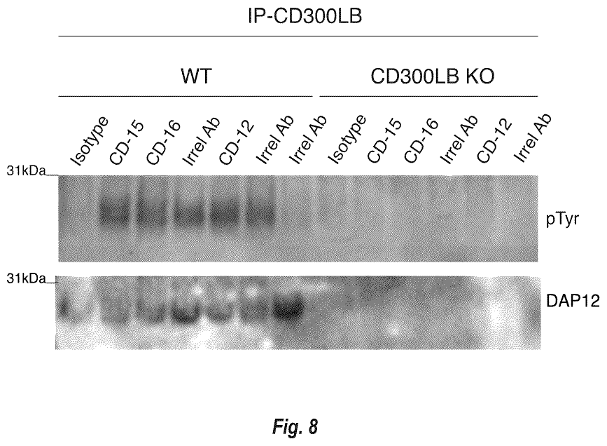

To determine the effects of anti-CD300LB antibodies of the present disclosure on proximal CD300LB signaling, DAP12 phosphorylation was measured following stimulation of wild type and CD300LB KO mouse bone marrow derived macrophages. Before stimulation, bone marrow derived macrophages were starved for 4 h in 1% serum RPMI. 15×10⁶ cells were then incubated in ice for 15 min with 1 ug of antibody for 1×10⁶ cells. Cells were then washed and incubated at 37° C. for the indicated period of time in the presence of goat anti-human IgG or goat-anti mouse IgG (1.5 ug for 1×10⁶ cells). After stimulation, cells were lysed with lysis buffer (1% n-Dodecyl-β-D-Maltoside, 50 Mm Tris-4C (pH 8.0), 150 mM NaCl, 1 mM EDTA, 1.5 nM MgCl₂, 10% glycerol, plus protease and phosphatase inhibitors) and immunoprecipitated with anti-CD300LB antibody (goat-anti mouse CD300LB, R&D Systems, #AF2580) and isotype control. Precipitated proteins were fractionated by SDS-PAGE, transferred to PVDF membranes and probed with anti-phosphotyrosine Ab (4G10, Millipore) and anti-DAP12 Ab (Cells Signaling). The results from these studies are presented in FIG. 8. In FIG. 8, Irrel Ab refers to a control (irrelevant) antibody.

As shown in FIG. 8, antibodies CD-15, CD-16 and CD-12 can agonize mCD300LB, activating the receptor and causing DAP12 tyrosine phosphorylation. As the detection of tyrosine phosphorylation is not specific to CD300LB activation, we further demonstrate that this signal is not detectable in CD300LB knockout cells and thus is specifically caused by the antibodies activating the CD300LB receptor.

### VII. Description of the Sequences

Amino Acid Sequences of the heavy chain variable regions and light chain variable regions of the antibodies disclosed herein are set forth in Table 13. The CDR sequences in Table 13 are underlined. The Kabat heavy chain HVR sequences of the antibodies disclosed herein are set forth in Table 14. The Kabat light chain HVR sequences of the antibodies disclosed herein are set forth in Table 15. Table 16 discloses particular sequences of the disclosure.

TABLE 13

| | Heavy Chain | SEQ ID | Light Chain | SEQ ID |
|---|---|---|---|---|
| Antibody | Variable Region | NO: | Variable Region | NO: |
| CD-01 | RVQLQQSGAELVRPGASVTLSC | 1 | DVVMTQSPLSLPVSLGDQASI | 2 |
| | KASGFTFTDFEMHWVKKTPVHG | | SCRSSQSLVHSDGNTYLYWYL | |
| | LEWIGAIDPESGNTAYNQKFRG | | QKPGQSPKLLIYKVSNRFSGV | |
| | KATLTADKSSSTAYMELRYLTS | | PDRFSGSGSGTDFTFKISRVG | |
| | EDSAVYYCTPLISGADYWGQGT | | AEDLGVYFCSQSTHVPWTFGG | |
| | TLTVSS | | GTKLEII | |
| CD-02 | QVQLQQPGAELVRPGSSVKLSC | 3 | ENVLTQSPAIMSASPGEKVTM | 4 |
| | QASGYTFTNYWMHWVRQRPIQG | | TCSASSSVSYIHWYQQKSSTS | |
| | LEWIGNIDPSDSETHYNQKFKD | | PKLWIYDTSKLASGVPGRFSG | |
| | RATLTVDKSSSTAYMQLSSLTS | | SGSGNSYSLTISSMETEDVAT | |
| | EDSAVYYCARSGVYDDEGGDYW | | YYCFQGSGYPLTFGAGTKLEL | |
| | GQGTTLTVSS | | K | |
| CD-03 | EVQLQQSGPELVKPGASVKISC | 5 | DVVMTQTPLTLSVTIGQPASI | 6 |
| | KASGYTFTDYYINWVRQSHGKS | | SCKSSQSLLDSDGKTYLNWLL | |
| | LECIGDINPNNGGTNYNQKFKG | | QRPGQSPKRLIYLVSKLDSGV | |
| | KATLTVDRSSNTAYMEFRSLTF | | PDRFTGSGSGTDFTLKISRVE | |
| | DDSALYYCAAEGAYWGQGTLVT | | AEDLGVYYCWQGTHEPWTFGG | |
| | VSA | | GTKLEIK | |
| CD-04 | QVQLQQSGAELVRPGASVTLSC | 7 | DVVMTQTPLSLPVSLGDQASI | 8 |
| | RASGYTFTDFEMHWVKQTPLHG | | SCRSSQTLVHNNGNTYLYWYL | |
| | LEWIGAIAPETGGTAYNQKFKG | | QKPGQSPKLLIYQVSNRFFGV | |
| | KAILTADKSSSTAYVELRSLTS | | PDRFSGSGSGTDFTLKISRVE | |
| | EDSAVYFCTRLGGMDYWGQGTS | | AEDLGLYFCSQSTHVPWTFGG | |
| | VTVSS | | GTKLEIK | |
| CD-05 | QVQLVQSGAEVKKPGASVKVSC | 9 | DIQMTQSPSSLSASVGDRVTI | 10 |
| | KASGYTFSRYYINWVRQAPGQG | | TCRASQNIGTYLNWYQQKPGK | |
| | LEWMGWINPNSGGTNYAQKFQG | | APKLLIYAASSLQSGVPSRFS | |
| | RVTMTRDTSTSTVYMELSSLRS | | GSGSGTDFTLTISSLQPEDFA | |
| | EDTAVYYCTTDQLRFGKTGPYY | | TYYCQQSYSDTLTFGGGTKVE | |
| | GMDVWGQGTTVTVSS | | IK | |
| CD-06 | QVQLVQSGAEVKKPGASVKVSC | 11 | DIQMTQSPSSLSASVGDRVTI | 12 |
| | KASGYTFTTYAVHWVRQAPGQG | | TCRASQSISSYLNWYQQKPGK | |
| | LEWMGIVNPNSGGTNYAQKFQG | | APKLLIYATSTLQSGVPSRFS | |
| | RVTMTRDTSTSTVYMELSSLRS | | GSGSGTDFTLTISSLQPEDFA | |
| | EDTAVYYCARDIVPMNNFYNYY | | TYYCQQSYRIPYTFGQGTKVE | |
| | YGMDVWGQGTTVTVSS | | IK | |
| CD-07 | QVQLVQSGAEVKKPGASVKVSC | 13 | DIQMTQSPSSLSASVGDRVTI | 14 |
| | KASGYTFTNYRMHWVRQAPGQG | | TCQASQDISNYLNWYQQKPGK | |
| | LEWMGWINPNSGGTIYAQKFQG | | APKLLIYGASSLQSGVPSRFS | |
| | RVTMTRDTSTSTVYMELSSLRS | | GSGSGTDFTLTISSLQPEDFA | |
| | EDTAVYYCARDLDYWGQGTLVT | | TYYCQQSYSTPWTFGPGTKVE | |
| | VSS | | IK | |
| CD-08 | EVQLLESGGGLVQPGGSLRLSC | 15 | DIQMTQSPSSLSASVGDRVTI | 16 |
| | AASGFTFSNAWMSWVRQAPGKG | | TCQASQDINNYVNWYQQKPGK | |
| | LEWVSVISGSGGRTYYADSVKG | | APKLLIYAASNLQSGVPSRFS | |
| | RFTISRDNSKNTLYLQMNSLRA | | GSGSGTDFTLTISSLQPEDFA | |
| | EDTAVYYCARDGTTVAPKGMDV | | TYYCQQSYSPPYTFGQGTKLE | |
| | WGQGTTVTVSS | | IK | |
| CD-09 | QVQLVQSGAEVKKPGASVKVSC | 17 | DIQMTQSPSSLSASVGDRVTI | 12 |
| | KASGYTFTTYAVHWVRQAPGQG | | TCRASQSISSYLNWYQQKPGK | |
| | LEWMGIINPNSGGTNYAQKFQG | | APKLLIYATSTLQSGVPSRFS | |
| | RVTMTRDTSTSTVYMELSSLRS | | GSGSGTDFTLTISSLQPEDFA | |
| | EDTAVYYCARDIVPMNNFYNYY | | TYYCQQSYRIPYTFGQGTKVE | |
| | YGMDVWGQGTTVTVSS | | IK | |
| CD-10 | QVQLQQSGPELVRPGVSVKISC | 18 | QIVLTQSPAIMSASPGEKVTM | 19 |
| | KGSGYTFTDYAMHWVKQSHVRS | | TCSASSSVNYMHWYQQKSGTS | |
| | REWIGVIGTYNGHTNYNQKFKG | | PKRWIYDTSKLSSGVPARFSG | |
| | KATMTVDKSSSTAYLELARLTS | | SGSGTSYSLTISTMEAEDAAT | |
| | EDSAIYYCARSGSYYYSMDYWG | | YYCQQWSSNPPMTFGGGTKLE | |
| | QGTSVTVSS | | IK | |
| CD-11 | QIQLVQSGPEVKKPGETVKISC | 20 | DIVLTQSPPSLAVSLGQRATI | 21 |
| | KASGYTLTDYSMHWVMQTPGTG | | SCRATKSVSPYLHWYQQTPGQ | |
| | LKWMAWMNTETGEPTYADDFKG | | PPKLLIYLASNLDSGVPARFS | |
| | RFAFSLETSAGTAYLQIDNLKN | | GSGSGTDFTLNIHPVEEEDAA | |
| | EDTATYFCATYGKSYGMKYWGQ | | TYYCQHNKELPPTFGGGTRLE | |
| | GTSVTVSS | | IK | |
| CD-12 | QIQLVQSGPELKKPGETVKISC | 22 | DIVLTQSPASLPVSLGQRATI | 23 |
| | KASGYTFTDYSMHWVRQAPGKG | | SCRASKSVRTSGASYMHWYQQ | |
| | LKWMAWINTETGEATHADDFKG | | KPGQPPKLLIYLASNLKSGVP | |
| | RFDFSLDTSASTAYLQIKNLKN | | TRFSGSGSGTDFTLNIHPVEE | |
| | EDTGIYFCVCYGSSYGMDYWGQ | | EDAATYYCQHNREVPPTFGGG | |
| | GTSVTVSS | | TKLEIK | |
| CD-13 | QIQLVQSGPELKKPGETVKISC | 24 | DIVLTQSPASLSVSLGQRATI | 25 |
| | KASGYTFTDYSMHWVKQAPGKG | | SCRASKSVGSSGSSYLHWYQQ | |
| | FKWMAWINTETGAPTHADDFRG | | KPGQPPKLLIYLASNLESGVP | |
| | RFAFSLETSASTAFLQISNLKT | | ARFSGSGSGTEFTLNIHPVEE | |
| | EDTATYFCVTFGKSYGMNYWGQ | | EDAATYYCQHNAEVPPTFGGG | |
| | GTSVTVSS | | TKLEIK | |
| CD-14 | QIQLVQSGPELKKPGETVKISC | 26 | DIVMTQSQKFMSTSVGDRVSV | 27 |
| | KASGYTFTDYSLHWVKQTPEKN | | TCKASQNVGIRVAWHQQKPGQ | |
| | LKWMAWINTETGAPTYADDFKG | | SPKTLIYSTSYRYSGVPDRFT | |
| | RFAFSLETSSNTAYLLINNLKN | | GSGSGTEFILTISNVQSEDLA | |
| | EDTATYFCALTMVNYWGQGTTL | | EYFCQQYNSYPYTFGGGTKLE | |
| | TVSS | | IK | |
| CD-15 | QIQLVQSGPELKKPGETVKISC | 28 | DIVLTQSPASLPVSLGQRATI | 29 |
| | QVSGYTFTDYSMHWLKQAPGKG | | SCRASKSVSTSGSSYLHWYQQ | |
| | LKWMAWINTETGESTYTDEFKG | | KPGQPPKLLIYLASKLESGVP | |
| | RFVFSLETSVRTAYLKINNLKN | | ARFRGSGSGTDFTLNIHPVEE | |
| | EDTATYFCGTYGSSYGMAYWGQ | | EDAATYYCQHNREVPPTFGGG | |
| | GTSVTVSS | | TKLEIK | |
| CD-16 | QIQLVQSGPELKKPGETVKISC | 30 | DIVLTQSPASLAVSLGQRATI | 31 |
| | KASGYTFTDYSMHWVKQAPGKG | | SCRASKSVSRSGYSYLHWYQQ | |
| | LKWMAWINTETGEPTYADDFRG | | KPGQPPKLLIYLASKLESGVP | |
| | RFAFSLETSGRTAYLEINNLRN | | ARFSGSGSGTDFTLNIHPVEA | |
| | EDTATYFCVTYGSSYGMAYWGQ | | EDAATYYCQHNREIPPTFGGG | |
| | GTSVTVSS | | TKLEIK | |
| CD-17 | QIQLVQSGPELKKPGETVKISC | 32 | DIVLTQSPASLAVSLGQRATI | 33 |
| | KASGYTLTDYSMHWVKQAPGKG | | SCRASKSVSPSGHSYMHWYQQ | |
| | LKWMGWINTETGEPRYGDDFKG | | KPGQPPKLLIYLASNLKSGVP | |
| | RFAFSLETSASSAYLQINNLKN | | ARFSGSGSGTDFTLNIHPVEE | |
| | DDTATYFCATYGRSYGMDFWGQ | | GDAATYYCQHNRELPPTFGGG | |
| | GTSVTVSS | | SKLEIK | |
| CD-18 | DVQLQESGPDLVKPSQSLSLTC | 34 | DVLMTQTPLSLPVSLGDQASI | 35 |
| | TVTGYSITSGYSWHWIRQFPDN | | SCRSSQSIVHSNGNTYLEWYL | |
| | KLEWMGYILSRGDTNFNPSLKS | | QKPGQSPKLLIYKVSDRFSGV | |
| | RISITRSTSNNQFFLHENSVTT | | PDRFSGSGSGTDFTLKISRVE | |
| | EDTATYYCARDFFDYWGQGTTL | | AEDLGVYFCFQGSHVPYTFGG | |
| | TVSS | | GTKLEIK | |

TABLE 14

| | | SEQ ID | | SEQ ID | | SEQ ID |
|---|---|---|---|---|---|---|
| Antibody | HVR-H1 | NO: | HVR-H2 | NO: | HVR-H3 | NO: |
| CD-01 | DFEMH | 36 | AIDPESGNT | 47 | LISGADY | 65 |
| | | | AYNQKFRG | | | |
| CD-02 | NYWMH | 37 | NIDPSDSET | 48 | SGVYDDEGGD | 66 |
| | | | HYNQKFKD | | Y | |
| CD-03 | DYYIN | 38 | DINPNNGGT | 49 | EGAY | 67 |
| | | | NYNQKFKG | | | |
| CD-04 | DFEMH | 36 | AIAPETGGT | 50 | LGGMDY | 68 |
| | | | AYNQKFKG | | | |
| CD-05 | RYYIN | 39 | WINPNSGGT | 51 | DQLRFGKTGP | 69 |
| | | | NYAQKFQG | | YYGMDV | |
| CD-06 | TYAVH | 40 | IVNPNSGGT | 52 | DIVPMNNFYN | 70 |
| | | | NYAQKFQG | | YYYGMDV | |
| CD-07 | NYRMH | 41 | WINPNSGGT | 53 | DLDY | 71 |
| | | | IYAQKFQG | | | |
| CD-08 | NAWMS | 42 | VISGSGGRT | 54 | DGTTVAPKGM | 72 |
| | | | YYADSVKG | | DV | |
| CD-09 | TYAVH | 40 | IINPNSGGT | 55 | DIVPMNNFYN | 70 |
| | | | NYAQKFQG | | YYYGMDV | |
| CD-10 | DYAMH | 43 | VIGTYNGHT | 56 | SGSYYYSMDY | 73 |
| | | | NYNQKFKG | | | |
| CD-11 | DYSMH | 44 | WMNTETGEP | 57 | YGKSYGMKY | 74 |
| | | | TYADDFKG | | | |
| CD-12 | DYSMH | 44 | WINTETGEA | 58 | FGKSYGMNY | 75 |
| | | | THADDFKG | | | |
| CD-13 | DYSMH | 44 | WINTETGAP | 59 | FGKSYGMNY | 75 |
| | | | THADDFRG | | | |
| CD-14 | DYSLH | 45 | WINTETGAP | 60 | TMVNY | 76 |
| | | | TYADDFKG | | | |
| CD-15 | DYSMH | 44 | WINTETGES | 61 | YGSSYGMAY | 77 |
| | | | TYTDEFKG | | | |
| CD-16 | DYSMH | 44 | WINTETGEP | 62 | YGSSYGMAY | 77 |
| | | | TYADDFRG | | | |
| CD-17 | DYSMH | 44 | WINTETGEP | 63 | YGRSYGMDF | 78 |
| | | | RYGDDFKG | | | |
| CD-18 | SGYSWH | 46 | YILSRGDTN | 64 | DEFDY | 79 |
| | | | FNPSLKS | | | |

TABLE 15

| | | SEQ ID | | SEQ ID | | SEQ ID |
|---|---|---|---|---|---|---|
| Antibody | HVR-L1 | NO: | HVR-L2 | NO: | HVR-L3 | NO: |
| CD-01 | RSSQSLVHS | 80 | KVSNRFS | 97 | SQSTHVPWT | 112 |
| | DGNTYLY | | | | | |
| CD-02 | SASSSVSYI | 81 | DTSKLAS | 98 | FQGSGYPLT | 113 |
| | H | | | | | |
| CD-03 | KSSQSLLDS | 82 | LVSKLDS | 99 | WQGTHFPWT | 114 |
| | DGKTYLN | | | | | |
| CD-04 | RSSQTLVHN | 83 | QVSNRFF | 100 | SQSTHVPWT | 112 |
| | NGNTYLY | | | | | |
| CD-05 | RASQNIGTY | 84 | AASSLQS | 101 | QQSYSDTLT | 115 |
| | LN | | | | | |
| CD-06 | RASQSISSY | 85 | ATSTLQS | 102 | QQSYRIPYT | 116 |
| | LN | | | | | |
| CD-07 | QASQDISNY | 86 | GASSLQS | 103 | QQSYSTPWT | 117 |
| | IN | | | | | |
| CD-08 | QASQDINNY | 87 | AASNLQS | 104 | QQSYSPPYT | 118 |
| | VN | | | | | |
| CD-09 | RASQSISSY | 85 | ATSTLQS | 102 | QQSYRIPYT | 116 |
| | LN | | | | | |
| CD-10 | SASSSVNYM | 88 | DTSKLSS | 105 | QQWSSNPPMT | 119 |
| | H | | | | | |
| CD-11 | RATKSVSPY | 89 | LASNLDS | 106 | QHNKELPPT | 120 |
| | LH | | | | | |
| CD-12 | RASKSVRTS | 90 | LASNLKS | 107 | QHNREVPPT | 121 |
| | GASYMH | | | | | |
| CD-13 | RASKSVGSS | 91 | LASNLES | 108 | QHNAEVPPT | 122 |
| | GSSYLH | | | | | |
| CD-14 | KASQNVGIR | 92 | STSYRYS | 109 | QQYNSYPYT | 123 |
| | VA | | | | | |
| CD-15 | RASKSVSTS | 93 | LASKLES | 110 | QHNREVPPT | 121 |
| | GSSYLH | | | | | |
| CD-16 | RASKSVSRS | 94 | LASKLES | 110 | QHNREIPPT | 124 |
| | GYSYLH | | | | | |
| CD-17 | RASKSVSPS | 95 | LASNLKS | 107 | QHNRELPPT | 125 |
| | GHSYMH | | | | | |
| CD-18 | RSSQSIVHS | 96 | KVSDRFS | 111 | FQGSHVPYT | 126 |
| | NGNTYLE | | | | | |

TABLE 16

| SEQ | | |
|---|---|---|
| ID | | |
| NO: | Sequence Title | Sequence |
| 127 | Human CD300LB | MWLPPALLLLSLSGCFSIQGPESVRAPEQGSLTVQCHYKQGWE |
| | NP_777552 | TYIKWWCRGVRWDTCKILIETRGSEQGEKSDRVSIKDNQKDRT |
| | | FTVTMEGLRRDDADVYWCGIERRGPDLGTQVKVIVDPEGAAST |
| | | TASSPTNSNMAVFIGSHKRNHYMLLVFVKVPILLILVTAILWL |
| | | KGSQRVPEEPGEQPIYMNFSEPLTKDMAT |
| 128 | Mouse CD300LB | MWLSPALLLLSFPGCLSIQGPALVRGPEQGSVTVQCRYSSRWQ |
| | NP_954691.3 | TNKKWWCRGASWSTCRVLIRSTGSEKETKSGRLSIRDNQKNHS |
| | | FQVTMEMLRQNDTDTYWCGIEKFGTDRGTRVKVNVYSVGKDTM |
| | | STSNQLPWPTVDGSTDMVSSDLQKRTYYMLLVFVKVPALLILV |
| | | GAVLWLKRSTQKVPEEQWRHTLCSDLDSELLAKDISP |
| 129 | Cynomolgus | MWLSPALLLLSLSGCFSIRGPVSVRGPEKGSVKVQCHYKRGWE |
| | CD300LB | THVKWWCRGARWVSCEILIQTRGSEQEEKKDLVSIKD |
| | XP_001090960.2 | SHTDCTFTVTMEGLRQNDSNIYWCGIQRRGPDLGTQVQLTVDP |
| | | EGAASAPANSSVNSNIRVSISTHKRNHYMLLVFVKVP |
| | | ILLILVGAILWLKGFQRVPEEPGDQPIYMNESELLTKDMAA |
| 130 | Human CD300LB | MWLPPALLLLSLSGCFSIQGPESVRAPEQGSLTVQCHYKQGWE |
| | Extracellular Domain | TYIKWWCRGVRWDTCKILIETRGSEQGEKSDRVSIKDNQKDRT |
| | | FTVTMEGLRRDDADVYWCGIERRGPDLGTQVKVIVDPEGAAST |
| | | TASSPTNSNMAVFIGSHKRNHY |
| 131 | Mouse CD300LB | MWLSPALLLLSFPGCLSIQGPALVRGPEQGSVTVQCRYSSRWQ |
| | Extracellular Domain | TNKKWWCRGASWSTCRVLIRSTGSEKETKSGRLSIRD |
| | | NQKNHSFQVTMEMLRQNDTDTYWCGIEKFGTDRGTRVKVNVYS |
| | | VGKDTMSTSNQLPWPTVDGSTDMVSSDLQKRT |
| 132 | Human DAP12 | MGGLEPCSRLLLLPLLLAVSGLRPVQAQAQSDCSCSTVSPGVL |
| | NP_003323 | AGIVMGDLVLTVLIALAVYFLGRLVPRGRGAAEAATRKQRITE |
| | | TESPYQELQGQRSDVYSDLNTQRPYYK |
| 133 | Human CD300LB- | MWLPPALLLLSLSGCFSIQGPESVRAPEQGSLTVQCHYKQGWE |
| | DAP12 chimera | TYIKWWCRGVRWDTCKILIETRGSEQGEKSDRVSIKD |
| | | NQKDRTFTVTMEGLRRDDADVYWCGIERRGPDLGTQVKVIVDP |
| | | EGAASTTASSPTNSNMAVFIGSHKRNQAQSDCSCSTV |
| | | SPGVLAGIVMGaLVLTVLIALAVYFLGRLVPRGRGAAEAATRK |
| | | QRITETESPYQELQGQRSDVYSDLNTQRPYYK |
| 134 | Mouse CD300LB- | MWLSPALLLLSFPGCLSIQGPALVRGPEQGSVTVQCRYSSRWQ |
| | DAP12 chimera | TNKKWWCRGASWSTCRVLIRSTGSEKETKSGRLSIRD |
| | | NQKNHSFQVTMEMLRQNDTDTYWCGIEKFGTDRGTRVKVNVYS |
| | | VGKDTMSTSNQLPWPTVDGSTDMVSSDLQKRTQSDTE |
| | | PRCDCSSVSPGVLAGIVLGaLVLTLLIALAVYSLGRLVSRGQG |
| | | TAEGTRKQHIAETESPYQELQGQRPEVYSDLNTQRQY |
| | | YR |
| 135 | Human | MGWSCIILFLVATATGVHSEQKLISEEDLLSKCRTVAGPVGGS |
| | CD300ANM_007261 | LSVQCPYEKEHRTLNKYWCRPPQIFLCDKIVETKGSAGKRNGR |
| | CDS | VSIRDSPANLSFTVTLENLTEEDAGTYWCGVDTPWLRDEHDPV |
| | (Signal Sequence | VEVEVSVFPASTSMTPASITAAKTSTITTAFPPVSSTTLFAVG |
| | underlined; | ATHSASIQEETEEVVNSQLPLLLSLLALLLLLLVGASLLAWRM |
| | Myc-tag in italics) | FQKWIKAGDHSELSQNPKQAATQSELHYANLELLMWPLQEKPA |
| | | PPREVEVEYSTVASPREELHYASVVEDSNTNRIAAQRPREEEP |
| | | DSDYSVIRKT |
| 136 | Human CD300C- | MGWSCIILFLVATATGVHSEQKLISEEDLMTVAGPVGGSLSVQ |
| | DAP12 chimera | CRYEKEHRTLNKFWCRPPQILRCDKIVETKGSAGKRNGRVSIR |
| | NM 006678 CDS | DSPANLSFTVTLENLTEEDAGTYWCGVDTPWLRDEHDPIVEVE |
| | (Signal Sequence | VSVFPAGTTTASSPQSSMGTSGPPTKLPVHTWPSVTRKDSPEP |
| | underlined; | SPHPGSLFSNVRQAQSDCSCSTVSPGVLAGIVMGALVLTVLIA |
| | Myc-tag in italics) | LAVYFLGRLVPRGRGAAEAATRKQRITETESPYQELQGQRSDV |
| | | YSDLNTQRPYYK |
| 137 | Human CD300LD- | MGWSCIILFLVATATGVHSEQKLISEEDLAKITGPTTVNGSEQ |
| | DAP12 chimera | GSLTVQCAYGSGWETYLKWRCQGADWNYCNILVKTNGSEQEVK |
| | NM_001115152 CDS | KNRVSIRDNQKNHVFTVTMENLKRDDADSYWCGTERPGIDLGV |
| | (Signal Sequence | KVQVTINPGTQTAVSEWTTTTASLAFTAAATQKTSSPLTRSPL |
| | underlined; | KSTHQAQSDCSCSTVSPGVLAGIVMGALVLTVLIALAVYFLGR |
| | Myc-tag in italics) | LVPRGRGAAEAATRKQRITETESPYQELQGQRSDVYSDLNTQR |
| | | PYYK |
| 138 | Human CD300E- | MGWSCIILFLVATATGVHSEQKLISEEDLLKGPGSVTGTAGDS |
| | DAP12 chimera | LTVWCQYESMYKGYNKYWCRGQYDTSCESIVETKGEEKVERNG |
| | NM_181449 | RVSIRDHPEALAFTVTMQNLNEDDAGSYWCKIQTVWVLDSWSR |
| | (Signal Sequence | DPSDLVRVYVSPAITTPRRTTHPATPPIFLVVNPGRNLSTGEV |
| | underlined; | LTQNSGFRLSSPHQAQSDCSCSTVSPGVLAGIVMGALVLTVLI |
| | Myc-tag in italics) | ALAVYFLGRLVPRGRG |
| 139 | Human CD300F | MGWSCIILFLVATATGVHSEQKLISEEDLTQITGPTTVNGLER |
| | NM_139018 | GSLTVQCVYRSGWETYLKWWCRGAIWRDCKILVKTSGSEQEVK |
| | (Signal Sequence | RDRVSIKDNQKNRTFTVTMEDLMKTDADTYWCGIEKTGNDLGV |
| | underlined; | TVQVTIDPAPVTQEETSSSPTLTGHHLDNRHKLLKLSVLLPLI |
| | Myc-tag in italics | FTILLLLLVAASLLAWRMMKYQQKAAGMSPEQVLQPLEGDLCY |
| | CDS) | ADLTLQLAGTSPQKATTKLSSAQVDQVEVEYVTMASLPKEDIS |
| | | YASLTLGAEDQEPTYCNMGHLSSHLPGRGPEEPTEYSTISRP |
| 140 | Human CD300G | MGWSCIILFLVATATGVHSEQKLISEEDLLEGPEEISGFEGDT |
| | NM_145273 | VSLQCTYREELRDHRKYWCRKGGILFSRCSGTIYAEEEGQETM |
| | (Signal Sequence | KGRVSIRDSRQELSLIVTLWNLTLQDAGEYWCGVEKRGPDESL |
| | underlined; | LISLFVFPGPCCPPSPSPTFQPLATTRLQPKAKAQQTQPPGLT |
| | Myc-tag in italics) | SPGLYPAATTAKQGKTGAEAPPLPGTSQYGHERTSQYTGTSPH |
| | | PATSPPAGSSRPPMQLDSTSAEDTSPALSSGSSKPRVSIPMVR |
| | | ILAPVLVLLSLLSAAGLIAFCSHLLLWRKEAQQATETQRNEKF |
| | | CLSRLTAEEKEAPSQAPEGDVISMPPLHTSEEELGFSKFVSA |
| 141 | Murine CD300A | MTQLASAVWLPTLLLLLLLFWLPGCVPLHGPSTMSGSVGESLS |
| | NM_170758 | VSCRYEEKFKTKDKYWCRVSLKILCKDIVKTSSSEEARSGRVT |
| | | IRDHPDNLTFTVTYESLTLEDADTYMCAVDISLEDGSLGEDKY |
| | | FKIELSVVPSEDPVSSPGPTLETPVVSTSLPTKGPALGSNTEG |
| | | HREHDYSQGLRLPALLSVLALLLFLLVGTSLLAWRMFQKRLVK |
| | | ADRHPELSQNLRQASEQNECQYVNLQLHTWSLREEPVLPSQVE |
| | | VVEYSTLALPQEELHYSSVAFN |
| | | SQRQDSHANGDSLHQPQDQKAEYSEIQKPRKGLSDLYL |
| 142 | Murine CD300LD | MIPRVIRLWLPSALFLSQVPGCVPLHGPSTITGAVGESLSVSC |
| | NM_145437 | QYEEKFKTKDKFWCRGSLKVLCKDIVKTSSSEEVRNGRVTIRD |
| | | HPDNLTFTVTYESLTLEDADTYMCAVDISLEDGSLGEDKYFKI |
| | | ELSVVPSEDPVTGSSLESGRDILESPTSSVGHTHPSVTTDDTI |
| | | PAPCPQPRSLRSSLYFWVLVSLKLFLFLSMLGAVLWVNRPQRC |
| | | SGGSSSRPCYENQ |
| 143 | Murine CD300F | MHLSLLVPFLFWITGCCTAEDPVTGPEEVSGQEQGSLTVQCRY |
| | NM_145634 | TSGWKDYKKYWCQGVPQRSCKTLVETDASEQLVKKNRVSIRDN |
| | | QRDFIFTVTMEDLRMSDAGIYWCGITKGGLDPMFKVTVNIGPA |
| | | IQVPITVPTMPPITSTTTIFTVTTTVKETSMFPTLTSYYSDNG |
| | | HGGGDSGGGEDGVGDGFLDLSVLLPVISAVLLLLLLVASLFAW |
| | | RMVRRQKKAAGPPSEQAQSLEGDLCYADLSLKQPRTSPGSSWK |
| | | KGSSMSSSGKDHQEEVEYVTMA |
| | | PFPREEVSYAALTLAGLGQEPTYGNTGCPITHVPRTGLEEETT |
| | | EYSSIRRPLPAAMP |
| 144 | Human CD300A-Fc | LSKCRTVAGPVGGSLSVQCPYEKEHRTLNKYWCRPPQIFLCDK |
| | | IVETKGSAGKRNGRVSIRDSPANLSFTVTLENLTEEDAGTYWC |
| | | GVDTPWLRDFHDPVVEVEVSVFPASTSMTPASITAAKTSTITT |
| | | AFPPVSSTTLFAVGATHSASIQEETEEVVNSQIEGRMDPKSCD |
| | | KTHTCPPCPAPEAEGAPSVELFPPKPKDTLMISRTPEVTCVVV |
| | | DVSHEDPEVKENWYVDGVEVHNAKTKPREEQYNSTYRVVSVLT |
| | | VLHQDWLNGKEYKCKVSNKALPAPIEKTISKAKGQPREPQVYT |
| | | LPPSRDELTKNQVSLTCLVKGFYPSDIAVEWESNGQPENNYKA |
| | | TPPVLDSDGSFFLYSKLTVDKSRWQQGNVFSCSVMHEALHNHY |
| | | TQKSLSLSPGK |
| 145 | Human CD300LB-Fc | IQGPESVRAPEQGSLTVQCHYKQGWETYIKWWCRGVRWDTCKI |
| | | LIETRGSEQGEKSDRVSIKDNQKDRTFTVTMEGLRRDDADVYW |
| | | CGIERRGPDLGTQVKVIVDPEGAASTTASSPTNSNMAVFIGSH |
| | | KRNHIEGRMDPKSCDKTHTCPPCPAPEAEGAPSVFLFPPKPKD |
| | | TLMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSRDELTKNQVSLTCLVKGFYPSDIA |
| | | VEWESNGQPENNYKATPPVLDSDGSFFLYSKLTVDKSRWQQGN |
| | | VFSCSVMHEALHNHYTQKSLSLSPGK |
| 146 | Human CD300C-Fc | MTVAGPVGGSLSVQCRYEKEHRTLNKFWCRPPQILRCDKIVET |
| | | KGSAGKRNGRVSIRDSPANLSFTVTLENLTEEDAGTYWCGVDT |
| | | PWLRDFHDPIVEVEVSVEPAGTTTASSPQSSMGTSGPPTKLPV |
| | | HTWPSVTRKDSPEPSPHPGSLFSNVRIEGRMDPKSCDKTHTCP |
| | | PCPAPEAEGAPSVFLFPPKPKDTLMISRTPEVTCVVVDVSHED |
| | | PEVKFNWYVDGVEVHNAKTKPREEQYNSTYRVVSVLTVLHQDW |
| | | LNGKEYKCKVSNKALPAPIEKTISKAKGQPREPQVYTLPPSRD |
| | | ELTKNQVSLTCLVKGFYPSDIAVEWESNGQPENNYKATPPVLD |
| | | SDGSFFLYSKLTVDKSRWQQGNVFSCSVMHEALHNHYTQKSLS |
| | | LSPGK |
| 147 | Human CD300D-Fc | AKITGPTTVNGSEQGSLTVQCAYGSGWETYLKWRCQGADWNYC |
| | | NILVKTNGSEQEVKKNRVSIRDNQKNHVFTVTMENLKRDDADS |
| | | YWCGTERPGIDLGVKVQVTINPGTQTAVSEWTTTTASLAFTAA |
| | | ATQKTSSPLTRSPLKSTHIEGRMDPKSCDKTHTCPPCPAPEAE |
| | | GAPSVFLFPPKPKDTLMISRTPEVTCVVVDVSHEDPEVKFNWY |
| | | VDGVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKATPPVLDSDGSFFLY |
| | | SKLTVDKSRWQQGNVFSCSVMHEALHNHYTQKSLSLSPGK |
| 148 | Human CD300E-Fc | LKGPGSVTGTAGDSLTVWCQYESMYKGYNKYWCRGQYDTSCES |
| | | IVETKGEEKVERNGRVSIRDHPEALAFTVTMQNLNEDDAGSYW |
| | | CKIQTVWVLDSWSRDPSDLVRVYVSPAITTPRRTTHPATPPIF |
| | | LVVNPGRNLSTGEVLTQNSGERLIEGRMDPKSCDKTHTCPPCP |
| | | APEAEGAPSVFLFPPKPKDTLMISRTPEVTCVVVDVSHEDPEV |
| | | KFNWYVDGVEVHNAKTKPREEQYNSTYRVVSVLTVLHQDWLNG |
| | | KEYKCKVSNKALPAPIEKTISKAKGQPREPQVYTLPPSRDELT |
| | | KNQVSLTCLVKGFYPSDIAVEWESNGQPENNYKATPPVLDSDG |
| | | SFFLYSKLTVDKSRWQQGNVFSCSVMHEALHNHYTQKSLSLSP |
| | | GK |
| 149 | Human CD300F-Fc | YSIATQITGPTTVNGLERGSLTVQCVYRSGWETYLKWWCRGAI |
| | | WRDCKILVKTSGSEQEVKRDRVSIKDNQKNRTFTVTMEDLMKT |
| | | DADTYWCGIEKTGNDLGVTVQVTIDPAPVTQEETSSSPTLTGH |
| | | HLDNRHKLLKLIEGRMDPKSCDKTHTCPPCPAPEAEGAPSVFL |
| | | FPPKPKDTLMISRTPEVTCVVVDVSHEDPEVKFNWYVDGVEVH |
| | | NAKTKPREEQYNSTYRVVSVLTVLHQDWLNGKEYKCKVSNKAL |
| | | PAPIEKTISKAKGQPREPQVYTLPPSRDELTKNQVSLTCLVKG |
| | | FYPSDIAVEWESNGQPENNYKATPPVLDSDGSFFLYSKLTVDK |
| | | SRWQQGNVFSCSVMHEALHNHYTQKSLSLSPGK |
| 150 | Mouse CD300A-Fc | LHGPSTMTGSVGQSLSVSCQYEEKFKTKDKYWCRGSLKVLCKD |
| | | IVKTSSSEEARSGRVTIRDHPDNLTFTVTYESLTLDDADTYMC |
| | | AVDIPFFNAPLGLDKYFKIELSVVPSEDPVSSPGPTLETPVVS |
| | | TSLPTKGPALGSNTEDRREHDYSQGLRIEGRMDPKSCDKTHTC |
| | | PPCPAPELLGGPSVFLFPPKPKDTLMISRTPEVTCVVVDVSHE |
| | | DPEVKFNWYVDGVEVHNAKTKPREEQYNSTYRVVSVLTVLHQD |
| | | WLNGKEYKCKVSNKALPAPIEKTISKAKGQPREPQVYTLPPSR |
| | | DELTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 151 | Mouse CD300LB-Fc | IQGPALVRGPEQGSVTVQCRYSSRWQTNKKWWCRGASWSTCRV |
| | | LIRSTGSEKETKSGRLSIRDNQKNHSFQVTMEMLRQNDTDTYW |
| | | CGIEKFGTDRGTRVKVNVYSVGKDTMSTSNQLPWPTVDGSTDM |
| | | VSSDLQKRTYYIEGRMDPEPRGPTIKPCPPCKCPAPNLLGGPS |
| | | VFIFPPKIKDVLMISLSPIVTCVVVDVSEDDPDVQISWFVNNV |
| | | EVHTAQTQTHREDYNSTLRVVSALPIQHQDWMSGKEFKCKVNN |
| | | KDLPAPIERTISKPKGSVRAPQVYVLPPPEEEMTKKQVTLTCM |
| | | VTDFMPEDIYVEWTNNGKTELNYKNTEPVLDSDGSYFMYSKLR |
| | | VEKKNWVERNSYSCSVVHEGLHNHHTTKSFSRTPGK |
| 152 | Mouse CD300C-Fc | HFPVRGPSTVTGTVGESLSVSCQYEKKLKTKKKIWCKWKSNVL |
| | | CKDIVKTSASEEARNGRVSIRDHPDNLTFTVTLENLTLEDAGT |
| | | YMCMVDIGFFYDAYLQIDKSFKVEVFVVPGKPPFKGSRPGNGI |
| | | NILASPTSSAVHTQPNVTTDDTIPAPSPELRSLLSSPIEGRMD |
| | | PEPRGPTIKPCPPCKCPAPNLLGGPSVFIFPPKIKDVLMISLS |
| | | PIVTCVVVDVSEDDPDVQISWFVNNVEVHTAQTQTHREDYNST |
| | | LRVVSALPIQHQDWMSGKEFKCKVNNKDLPAPIERTISKPKGS |
| | | VRAPQVYVLPPPEEEMTKKQVTLTCMVTDEMPEDIYVEWTNNG |
| | | KTELNYKNTEPVLDSDGSYFMYSKLRVEKKNWVERNSYSCSVV |
| | | HEGLHNHHTTKSFSRTPGK |
| 153 | Mouse CD300D-Fc | NPVTGPEEVSGQEQGSLTVQCQYTSDWKDYKKYWCQGVPQKSC |
| | | VFLIETDKSEQLVKKNRVSIRDNQREFIFTVIMEDLRMSDAGI |
| | | YWCGITKAGYDPVFKVNVSINPAPKSSMMTTTATVLKSIQPSA |
| | | ENTGKEQVTQSKEVTQSRPHTRSLIEGRMDPEPRGPTIKPCPP |
| | | CKCPAPNLLGGPSVFIFPPKIKDVLMISLSPIVTCVVVDVSED |
| | | DPDVQISWFVNNVEVHTAQTQTHREDYNSTLRVVSALPIQHQD |
| | | WMSGKEFKCKVNNKDLPAPIERTISKPKGSVRAPQVYVLPPPE |
| | | EEMTKKQVTLTCMVTDEMPEDIYVEWTNNGKTELNYKNTEPVL |
| | | DSDGSYFMYSKLRVEKKNWVERNSYSCSVVHEGLHNHHTTKSF |
| | | SRTPGK |
| 154 | Mouse CD300E-Fc | LTGPGSVSGYVGGSLRVQCQYSPSYKGYMKYWCRGPHDTTCKT |
| | | IVETDGSEKEKRSGPVSIRDHAANSTITVIMEDLSEDDAGSYW |
| | | CKIQTSFIWDSWSRDPSVSVRVNVFPATTPTLPATTAILPLVN |
| | | SGQNLRISTNVMFIFQLWSLIEGRMDPEPRGPTIKPCPPCKCP |
| | | APNLLGGPSVFIFPPKIKDVLMISLSPIVTCVVVDVSEDDPDV |
| | | QISWFVNNVEVHTAQTQTHREDYNSTLRVVSALPIQHQDWMSG |
| | | KEFKCKVNNKDLPAPIERTISKPKGSVRAPQVYVLPPPEEEMT |
| | | KKQVTLTCMVTDEMPEDIYVEWTNNGKTELNYKNTEPVLDSDG |
| | | SYFMYSKLRVEKKNWVERNSYSCSVVHEGLHNHHTTKSFSRTP |
| | | GK |

What is claimed is:

1. An isolated antibody that specifically binds to CD300LB, wherein the antibody comprises a heavy chain variable region (VH) and a light chain variable region (VL), wherein the heavy chain variable region (VH) comprises:
a. an HVR-H1 comprising an amino acid sequence chosen from any one of SEQ ID NOs:36-46;
b. an HVR-H2 comprising an amino acid sequence chosen from any one of SEQ ID NOs:47-64; and
c. an HVR-H3 comprising an amino acid sequence chosen from any one of SEQ ID NOs:65-79.

2. The antibody of claim 1, wherein the light chain variable region (VL) comprises:
a. an HVR-L1 comprising an amino acid sequence chosen from any one of SEQ ID NOs:80-96;
b. an HVR-L2 comprising an amino acid sequence chosen from any one of SEQ ID NOs:97-111; and
c. an HVR-L3 comprising an amino acid sequence chosen from any one of SEQ ID NOs:112-126.

3. The antibody of claim 1 or 2, wherein the antibody comprises a VH that is at least 90%, at least 95%, at least 97%, or at least 99% identical to an amino acid sequence chosen from any one of SEQ ID NOs:1, 3, 5, 7, 9, 11, 13, 15, 17, 18, 20, 22, 24, 26, 28, 30, 32, and 34.

4. The antibody of any one of claims 1-3, wherein the antibody comprises a VL that is at least 90%, at least 95%, at least 97%, or at least 99% identical to an amino acid sequence chosen from any one of SEQ ID NOs:2, 4, 6, 8, 10, 12, 14, 16, 19, 21, 23, 25, 27, 29, 31, 33, and 35.

5. The antibody of any one of claims 1-4, wherein the VH comprises 1, 2, 3, 4, 5, 6, 7, 8, 9, or 10 amino acid substitutions, insertions, and/or deletions compared to an amino acid sequence chosen from any one of SEQ ID NOs:1, 3, 5, 7, 9, 11, 13, 15, 17, 18, 20, 22, 24, 26, 28, 30, 32, and 34.

6. The antibody of any one of claims 1-5, wherein the VL comprises 1, 2, 3, 4, 5, 6, 7, 8, 9, or 10 amino acid substitutions, insertions, and/or deletions compared to an amino acid sequence chosen from any one of SEQ ID NOs:2, 4, 6, 8, 10, 12, 14, 16, 19, f21, 23, 25, 27, 29, 31, 33, and 35.

7. The antibody of any one of claims 1-6, wherein the antibody comprises a VH comprising an amino acid sequence chosen from any one of SEQ ID NOs:1, 3, 5, 7, 9, 11, 13, 15, 17, 18, 20, 22, 24, 26, 28, 30, 32, and 34.

8. The antibody of any one of claims 1-7, wherein the antibody comprises a VL comprising an amino acid sequence chosen from any one of SEQ ID NOs:2, 4, 6, 8, 10, 12, 14, 16, 19, 21, 23, 25, 27, 29, 31, 33, and 35.

9. An isolated antibody that specifically binds to human CD300LB, wherein the antibody comprises a VH comprising HVR-H1, HVR-H2, and HVR-H3 and a VL comprising HVR-L1, HVR-L2, and HVR-L3 of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

10. The isolated antibody of claim 9, wherein the antibody comprises a VH and/or a VL at least 90%, at least 95%, at least 97%, or at least 99% identical to those of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

11. The isolated antibody of claim 9 or claim 10, wherein the antibody comprises the VH and/or the VL of any one of antibodies CD-01, CD-02, CD-03, CD-04, CD-05, CD-06, CD-07, CD-07, CD-09, CD-10, CD-11, CD-12, CD-13, CD-14, CD-15, CD-16, CD-17, and CD-18.

12. An isolated antibody that specifically binds to human CD300LB, wherein the antibody comprises:
a. a VH comprising an HVR-H1 comprising the amino acid sequence of SEQ ID NO:36, an HVR-H2 comprising the amino acid sequence of SEQ ID NO:47, an HVR-H3 comprising the amino acid sequence of SEQ ID NO:65; and a VL comprising an HVR-L1 comprising the amino acid sequence of SEQ ID NO:80, an HVR-L2 comprising the amino acid sequence of SEQ ID NO:97, and an HVR-L3 comprising the amino acid sequence of SEQ ID NO: 112;
b. a VH comprising an HVR-H1 comprising the amino acid sequence of SEQ ID NO:37, an HVR-H2 comprising the amino acid sequence of SEQ ID NO:48; an HVR-H3 comprising the amino acid sequence of SEQ ID NO:66, an HVR-L1 comprising the amino acid sequence of SEQ ID NO:81; and a VL comprising an HVR-L2 comprising the amino acid sequence of SEQ ID NO: 98, and an HVR-L3 comprising the amino acid sequence of SEQ ID NO: 113;
c. a VH comprising an HVR-H1 comprising the amino acid sequence of SEQ ID NO:38, an HVR-H2 comprising the amino acid sequence of SEQ ID NO:49, an HVR-H3 comprising the amino acid sequence of SEQ ID NO:67; and a VL comprising an HVR-L1 comprising the amino acid sequence of SEQ ID NO:82, an HVR-L2 comprising the amino acid sequence of SEQ ID NO:99, and an HVR-L3 comprising the amino acid sequence of SEQ ID NO: 114;
d. a VH comprising an HVR-H1 comprising the amino acid sequence of SEQ ID NO:36, an HVR-H2 comprising the amino acid sequence of SEQ ID NO:50, an HVR-H3 comprising the amino acid sequence of SEQ ID NO:68; and a VL comprising an HVR-L1 comprising the amino acid sequence of SEQ ID NO:83, an HVR-L2 comprising the amino acid sequence of SEQ ID NO: 100, and an HVR-L3 comprising the amino acid sequence of SEQ ID NO: 112;
e. a VH comprising an HVR-H1 comprising the amino acid sequence of SEQ ID NO:39, an HVR-H2 comprising the amino acid sequence of SEQ ID NO:51, an HVR-H3 comprising the amino acid sequence of SEQ ID NO:69; and a VL comprising an HVR-L1 comprising the amino acid sequence of SEQ ID NO:84, an HVR-L2 comprising the amino acid sequence of SEQ ID NO:101, and an HVR-L3 comprising the amino acid sequence of SEQ ID NO: 115;
f. a VH comprising an HVR-H1 comprising the amino acid sequence of SEQ ID NO:40, an HVR-H2 comprising the amino acid sequence of SEQ ID NO:52, an HVR-H3 comprising the amino acid sequence of SEQ ID NO:70; and a VL comprising an HVR-L1 comprising the amino acid sequence of SEQ ID NO:85, an HVR-L2 comprising the amino acid sequence of SEQ ID NO: 102; and an HVR-L3 comprising the amino acid sequence of SEQ ID NO: 116;
g. a VH comprising an HVR-H1 comprising the amino acid sequence of SEQ ID NO:41, an HVR-H2 comprising the amino acid sequence of SEQ ID NO:53, an HVR-H3 comprising the amino acid sequence of SEQ ID NO:71; and a VL comprising an HVR-L1 comprising the amino acid sequence of SEQ ID NO:86, an HVR-L2 comprising the amino acid sequence of SEQ ID NO: 103, and an HVR-L3 comprising the amino acid sequence of SEQ ID NO: 117;
h. a VH comprising an HVR-H1 comprising the amino acid sequence of SEQ ID NO:42, an HVR-H2 comprising the amino acid sequence of SEQ ID NO:54, an HVR-H3 comprising the amino acid sequence of SEQ ID NO:72; and a VL comprising an HVR-L1 comprising the amino acid sequence of SEQ ID NO:87, an HVR-L2 comprising the amino acid sequence of SEQ ID NO: 104, and an HVR-L3 comprising the amino acid sequence of SEQ ID NO: 118;
i. a VH comprising an HVR-H1 comprising the amino acid sequence of SEQ ID NO:40, an HVR-H2 comprising the amino acid sequence of SEQ ID NO:55, an HVR-H3 comprising the amino acid sequence of SEQ ID NO:70; and a VL comprising an HVR-L1 comprising the amino acid sequence of SEQ ID NO:85, an HVR-L2 comprising the amino acid sequence of SEQ ID NO: 102, and an HVR-L3 comprising the amino acid sequence of SEQ ID NO: 116;
j. a VH comprising an HVR-H1 comprising the amino acid sequence of SEQ ID NO:43, an HVR-H2 comprising the amino acid sequence of SEQ ID NO:56, an HVR-H3 comprising the amino acid sequence of SEQ ID NO:73; and a VL comprising an HVR-L1 comprising the amino acid sequence of SEQ ID NO:88, an HVR-L2 comprising the amino acid sequence of SEQ ID NO: 105, and an HVR-L3 comprising the amino acid sequence of SEQ ID NO: 119;
k. a VH comprising an HVR-H1 comprising the amino acid sequence of SEQ ID NO:44, an HVR-H2 comprising the amino acid sequence of SEQ ID NO:57, an HVR-H3 comprising the amino acid sequence of SEQ ID NO:74; and a VL comprising an HVR-L1 comprising the amino acid sequence of SEQ ID NO:89, an HVR-L2 comprising the amino acid sequence of SEQ ID NO: 106, and an HVR-L3 comprising the amino acid sequence of SEQ ID NO: 120;
l. a VH comprising an HVR-H1 comprising the amino acid sequence of SEQ ID NO:44, an HVR-H2 comprising the amino acid sequence of SEQ ID NO:58, an HVR-H3 comprising the amino acid sequence of SEQ ID NO:75; and a VL comprising an HVR-L1 comprising the amino acid sequence of SEQ ID NO:90, an HVR-L2 comprising the amino acid sequence of SEQ ID NO: 107, and an HVR-L3 comprising the amino acid sequence of SEQ ID NO:121;
m. a VH comprising an HVR-H1 comprising the amino acid sequence of SEQ ID NO:44, an HVR-H2 comprising the amino acid sequence of SEQ ID NO:59, an HVR-H3 comprising the amino acid sequence of SEQ ID NO:75; and a VL comprising an HVR-L1 comprising the amino acid sequence of SEQ ID NO:91, an HVR-L2 comprising the amino acid sequence of SEQ ID NO:108, and an HVR-L3 comprising the amino acid sequence of SEQ ID NO: 122;
n. a VH comprising an HVR-H1 comprising the amino acid sequence of SEQ ID NO:45, an HVR-H2 comprising the amino acid sequence of SEQ ID NO:60, an HVR-H3 comprising the amino acid sequence of SEQ ID NO:76; and a VL comprising an HVR-L1 comprising the amino acid sequence of SEQ ID NO:92, an HVR-L2 comprising the amino acid sequence of SEQ ID NO: 109, and an HVR-L3 comprising the amino acid sequence of SEQ ID NO: 123;
o. a VH comprising an HVR-H1 comprising the amino acid sequence of SEQ ID NO:44, an HVR-H2 comprising the amino acid sequence of SEQ ID NO:61, an HVR-H3 comprising the amino acid sequence of SEQ ID NO:77; and a VL comprising an HVR-L1 comprising the amino acid sequence of SEQ ID NO:93, an HVR-L2 comprising the amino acid sequence of SEQ ID NO:110, and an HVR-L3 comprising the amino acid sequence of SEQ ID NO:121;
p. a VH comprising an HVR-H1 comprising the amino acid sequence of SEQ ID NO:44, an HVR-H2 comprising the amino acid sequence of SEQ ID NO:62, an HVR-H3 comprising the amino acid sequence of SEQ ID NO:77; and a VL comprising an HVR-L1 comprising the amino acid sequence of SEQ ID NO:94, an HVR-L2 comprising the amino acid sequence of SEQ ID NO:110, and an HVR-L3 comprising the amino acid sequence of SEQ ID NO: 124;
q. a VH comprising an HVR-H1 comprising the amino acid sequence of SEQ ID NO:44, HVR-H2 comprising the amino acid sequence of SEQ ID NO:63, an HVR-H3 comprising the amino acid sequence of SEQ ID NO:78; and a VL comprising an HVR-L1 comprising the amino acid sequence of SEQ ID NO:95, an HVR-L2 comprising the amino acid sequence of SEQ ID NO: 107, and an HVR-L3 comprising the amino acid sequence of SEQ ID NO: 125; or
r. a VH comprising an HVR-H1 comprising the amino acid sequence of SEQ ID NO:46, an HVR-H2 comprising the amino acid sequence of SEQ ID NO:64, an HVR-H3 comprising the amino acid sequence of SEQ ID NO:79; and a VL comprising an HVR-L1 comprising the amino acid sequence of SEQ ID NO:96, an HVR-L2 comprising the amino acid sequence of SEQ ID NO:111, and an HVR-L3 comprising the amino acid sequence of SEQ ID NO: 126.

13. The antibody of claim 12, wherein the antibody comprises:
a. the HVRs of claim 12.a. and further comprises a VH that is at least 90%, at least 95%, at least 97%, or at least 99% identical to the amino acid sequence of SEQ ID NO: 1;
b. the HVRs of claim 12.b. and further comprises a VH that is at least 90%, at least 95%, at least 97%, or at least 99% identical to the amino acid sequence of SEQ ID NO:3;
c. the HVRs of claim 12.c. and further comprises a VH that is at least 90%, at least 95%, at least 97%, or at least 99% identical to the amino acid sequence of SEQ ID NO:5;
d. the HVRs of claim 12.d. and further comprises a VH that is at least 90%, at least 95%, at least 97%, or at least 99% identical to the amino acid sequence of SEQ ID NO:7;
e. the HVRs of claim 12.e. and further comprises a VH that is at least 90%, at least 95%, at least 97%, or at least 99% identical to the amino acid sequence of SEQ ID NO:9;
f. the HVRs of claim 12.f. and further comprises a VH that is at least 90%, at least 95%, at least 97%, or at least 99% identical to the amino acid sequence of SEQ ID NO:11;
g. the HVRs of claim 12.g. and further comprises a VH that is at least 90%, at least 95%, at least 97%, or at least 99% identical to the amino acid sequence of SEQ ID NO:13;
h. the HVRs of claim 12.h. and further comprises a VH that is at least 90%, at least 95%, at least 97%, or at least 99% identical to the amino acid sequence of SEQ ID NO:15;
i. the HVRs of claim 12.i. and further comprises a VH that is at least 90%, at least 95%, at least 97%, or at least 99% identical to the amino acid sequence of SEQ ID NO:17;
j. the HVRs of claim 12.j. and further comprises a VH that is at least 90%, at least 95%, at least 97%, or at least 99% identical to the amino acid sequence of SEQ ID NO:18;
k. the HVRs of claim 12.k. and further comprises a VH that is at least 90%, at least 95%, at least 97%, or at least 99% identical to the amino acid sequence of SEQ ID NO:20;
l. the HVRs of claim 12.l. and further comprises a VH that is at least 90%, at least 95%, at least 97%, or at least 99% identical to the amino acid sequence of SEQ ID NO:22;
m. the HVRs of claim 12.m. and further comprises a VH that is at least 90%, at least 95%, at least 97%, or at least 99% identical to the amino acid sequence of SEQ ID NO:24;
n. the HVRs of claim 12.n. and further comprises a VH that is at least 90%, at least 95%, at least 97%, or at least 99% identical to the amino acid sequence of SEQ ID NO:26;
o. the HVRs of claim 12.o. and further comprises a VH that is at least 90%, at least 95%, at least 97%, or at least 99% identical to the amino acid sequence of SEQ ID NO:28;
p. the HVRs of claim 12.p. and further comprises a VH that is at least 90%, at least 95%, at least 97%, or at least 99% identical to the amino acid sequence of SEQ ID NO:30;
q. the HVRs of claim 12.q. and further comprises a VH that is at least 90%, at least 95%, at least 97%, or at least 99% identical to the amino acid sequence of SEQ ID NO:32; or
r. the HVRs of claim 12.r. and further comprises a VH that is at least 90%, at least 95%, at least 97%, or at least 99% identical to the amino acid sequence of SEQ ID NO:34.

14. The antibody of claim 12 or 13, wherein the antibody comprises:
a. the HVRs of claim 12.a. and further comprises a VL that is at least 90%, at least 95%, at least 97%, or at least 99% identical to the amino acid sequence of SEQ ID NO: 2;
b. the HVRs of claim 12.b. and further comprises a VL that is at least 90%, at least 95%, at least 97%, or at least 99% identical to the amino acid sequence of SEQ ID NO: 4;
c. the HVRs of claim 12.c. and further comprises a VL that is at least 90%, at least 95%, at least 97%, or at least 99% identical to the amino acid sequence of SEQ ID NO: 6;
d. the HVRs of claim 12.d. and further comprises a VL that is at least 90%, at least 95%, at least 97%, or at least 99% identical to the amino acid sequence of SEQ ID NO: 8;
e. the HVRs of claim 12.e. and further comprises a VL that is at least 90%, at least 95%, at least 97%, or at least 99% identical to the amino acid sequence of SEQ ID NO: 10;
f. the HVRs of claim 12.f. and further comprises a VL that is at least 90%, at least 95%, at least 97%, or at least 99% identical to the amino acid sequence of SEQ ID NO: 12;
g. the HVRs of claim 12.g. and further comprises a VL that is at least 90%, at least 95%, at least 97%, or at least 99% identical to the amino acid sequence of SEQ ID NO: 14;
h. the HVRs of claim 12.h. and further comprises a VL that is at least 90%, at least 95%, at least 97%, or at least 99% identical to the amino acid sequence of SEQ ID NO: 16;
i. the HVRs of claim 12.i. and further comprises a VL that is at least 90%, at least 95%, at least 97%, or at least 99% identical to the amino acid sequence of SEQ ID NO: 12;
j. the HVRs of claim 12.j. and further comprises a VL that is at least 90%, at least 95%, at least 97%, or at least 99% identical to the amino acid sequence of SEQ ID NO: 19;
k. the HVRs of claim 12.k. and further comprises a VL that is at least 90%, at least 95%, at least 97%, or at least 99% identical to the amino acid sequence of SEQ ID NO: 21;
l. the HVRs of claim 12.l. and further comprises a VL that is at least 90%, at least 95%, at least 97%, or at least 99% identical to the amino acid sequence of SEQ ID NO: 23;
m. the HVRs of claim 12.m. and further comprises a VL that is at least 90%, at least 95%, at least 97%, or at least 99% identical to the amino acid sequence of SEQ ID NO: 25;
n. the HVRs of claim 12.n. and further comprises a VL that is at least 90%, at least 95%, at least 97%, or at least 99% identical to the amino acid sequence of SEQ ID NO: 27;
o. the HVRs of claim 12.o. and further comprises a VL that is at least 90%, at least 95%, at least 97%, or at least 99% identical to the amino acid sequence of SEQ ID NO: 29;
p. the HVRs of claim 12.p. and further comprises a VL that is at least 90%, at least 95%, at least 97%, or at least 99% identical to the amino acid sequence of SEQ ID NO: 31;
q. the HVRs of claim 12.q. and further comprises a VL that is at least 90%, at least 95%, at least 97%, or at least 99% identical to the amino acid sequence of SEQ ID NO: 33; or
r. the HVRs of claim 12.r. and further comprises a VL that is at least 90%, at least 95%, at least 97%, or at least 99% identical to the amino acid sequence of SEQ ID NO: 35.

15. The antibody of any one of claims 12-14, wherein the antibody comprises:
a. the HVRs of claim 12.a. and further comprises a VH comprising the amino acid sequence of SEQ ID NO:1;
b. the HVRs of claim 12.b. and further comprises a VH comprising the amino acid sequence of SEQ ID NO:3;
c. the HVRs of claim 12.c. and further comprises a VH comprising the amino acid sequence of SEQ ID NO:5;
d. the HVRs of claim 12.d. and further comprises a VH comprising the amino acid sequence of SEQ ID NO:7;
e. the HVRs of claim 12.e. and further comprises a VH comprising the amino acid sequence of SEQ ID NO:9;
f. the HVRs of claim 12.f. and further comprises a VH comprising the amino acid sequence of SEQ ID NO:11;
g. the HVRs of claim 12.g. and further comprises a VH comprising the amino acid sequence of SEQ ID NO:13;
h. the HVRs of claim 12.h. and further comprises a VH comprising the amino acid sequence of SEQ ID NO:15;
i. the HVRs of claim 12.i. and further comprises a VH comprising the amino acid sequence of SEQ ID NO:17;
j. the HVRs of claim 12.j. and further comprises a VH comprising the amino acid sequence of SEQ ID NO:18;
k. the HVRs of claim 12.k. and further comprises a VH comprising the amino acid sequence of SEQ ID NO:20;
l. the HVRs of claim 12.l. and further comprises a VH comprising the amino acid sequence of SEQ ID NO:22;
m. the HVRs of claim 12.m. and further comprises a VH comprising the amino acid sequence of SEQ ID NO:24;
n. the HVRs of claim 12.n. and further comprises a VH comprising the amino acid sequence of SEQ ID NO:26;
o. the HVRs of claim 12.o. and further comprises a VH comprising the amino acid sequence of SEQ ID NO:28;
p. the HVRs of claim 12.p. and further comprises a VH comprising the amino acid sequence of SEQ ID NO:30;
q. the HVRs of claim 12.q. and further comprises a VH comprising the amino acid sequence of SEQ ID NO:32; or
r. the HVRs of claim 12.r. and further comprises a VH comprising the amino acid sequence of SEQ ID NO:34.

16. The antibody of any one of claims 12-15, wherein the antibody comprises:
a. the HVRs of claim 12.a. and further comprises a VL comprising the amino acid sequence of SEQ ID NO: 2;
b. the HVRs of claim 12.b. and further comprises a VL comprising the amino acid sequence of SEQ ID NO: 4;
c. the HVRs of claim 12.c. and further comprises a VL comprising the amino acid sequence of SEQ ID NO: 6;
d. the HVRs of claim 12.d. and further comprises a VL comprising the amino acid sequence of SEQ ID NO: 8;
e. the HVRs of claim 12.e. and further comprises a VL comprising the amino acid sequence of SEQ ID NO: 10;
f. the HVRs of claim 12.f. and further comprises a VL comprising the amino acid sequence of SEQ ID NO: 12;
g. the HVRs of claim 12.g. and further comprises a VL comprising the amino acid sequence of SEQ ID NO: 14;
h. the HVRs of claim 12.h. and further comprises a VL comprising the amino acid sequence of SEQ ID NO: 16;
i. the HVRs of claim 12.i. and further comprises a VL comprising the amino acid sequence of SEQ ID NO: 12;
j. the HVRs of claim 12.j. and further comprises a VL comprising the amino acid sequence of SEQ ID NO: 19;
k. the HVRs of claim 12.k. and further comprises a VL comprising the amino acid sequence of SEQ ID NO: 21;
l. the HVRs of claim 12.l. and further comprises a VL comprising the amino acid sequence of SEQ ID NO: 23;
m. the HVRs of claim 12.m. and further comprises a VL comprising the amino acid sequence of SEQ ID NO: 25;
n. the HVRs of claim 12.n. and further comprises a VL comprising the amino acid sequence of SEQ ID NO: 27;
o. the HVRs of claim 12.o. and further comprises a VL comprising the amino acid sequence of SEQ ID NO: 29;
p. the HVRs of claim 12.p. and further comprises a VL comprising the amino acid sequence of SEQ ID NO: 31;
q. the HVRs of claim 12.q. and further comprises a VL comprising the amino acid sequence of SEQ ID NO: 33; or
r. the HVRs of claim 12.r. and further comprises a VL comprising the amino acid sequence of SEQ ID NO: 35.

17. The antibody of any one of claims 9-12, wherein the antibody comprises
a. a VH comprising the amino acid sequence of SEQ ID NO: 1 and a VL comprising the amino acid sequence of SEQ ID NO: 2 (as shown in Table 13);
b. a VH comprising the amino acid sequence of SEQ ID NO: 3 and a VL comprising the amino acid sequence of SEQ ID NO: 4 (as shown in Table 13);
c. a VH comprising the amino acid sequence of SEQ ID NO: 5 and a VL comprising the amino acid sequence of SEQ ID NO: 6 (as shown in Table 13);
d. a VH comprising the amino acid sequence of SEQ ID NO: 7 and a VL comprising the amino acid sequence of SEQ ID NO: 8 (as shown in Table 13);
e. a VH comprising the amino acid sequence of SEQ ID NO: 9 and a VL comprising the amino acid sequence of SEQ ID NO: 10 (as shown in Table 13);
f. a VH comprising the amino acid sequence of SEQ ID NO: 11 and a VL comprising the amino acid sequence of SEQ ID NO: 12 (as shown in Table 13);
g. a VH comprising the amino acid sequence of SEQ ID NO: 13 and a VL comprising the amino acid sequence of SEQ ID NO: 14 (as shown in Table 13);
h. a VH comprising the amino acid sequence of SEQ ID NO: 15 and a VL comprising the amino acid sequence of SEQ ID NO: 16 (as shown in Table 13);
i. a VH comprising the amino acid sequence of SEQ ID NO: 17 and a VL comprising the amino acid sequence of SEQ ID NO: 12 (as shown in Table 13);
j. a VH comprising the amino acid sequence of SEQ ID NO: 18 and a VL comprising the amino acid sequence of SEQ ID NO: 19 (as shown in Table 13);
k. a VH comprising the amino acid sequence of SEQ ID NO: 20 and a VL comprising the amino acid sequence of SEQ ID NO: 21 (as shown in Table 13);
l. a VH comprising the amino acid sequence of SEQ ID NO: 22 and a VL comprising the amino acid sequence of SEQ ID NO: 23 (as shown in Table 13);
m. a VH comprising the amino acid sequence of SEQ ID NO: 24 and a VL comprising the amino acid sequence of SEQ ID NO: 25 (as shown in Table 13);
n. a VH comprising the amino acid sequence of SEQ ID NO: 26 and a VL comprising the amino acid sequence of SEQ ID NO: 27 (as shown in Table 13);
o. a VH comprising the amino acid sequence of SEQ ID NO: 28 and a VL comprising the amino acid sequence of SEQ ID NO: 29 (as shown in Table 13);
p. a VH comprising the amino acid sequence of SEQ ID NO: 30 and a VL comprising the amino acid sequence of SEQ ID NO: 31 (as shown in Table 13);
q. a VH comprising the amino acid sequence of SEQ ID NO: 32 and a VL comprising the amino acid sequence of SEQ ID NO: 33 (as shown in Table 13); or
r. a VH comprising the amino acid sequence of SEQ ID NO: 34 and a VL comprising the amino acid sequence of SEQ ID NO: 35 (as shown in Table 13).

18. An isolated antibody that specifically binds to human CD300LB, wherein the antibody comprises:
a. a VH and VL, wherein the VH comprises the HVR-H1, HVR-H2, and HVR-H3 of the antibody CD-01 (as shown in Table 14) and the VL comprises the HVR-L1, HVR-L2, and HVR-L3 of the antibody CD-01 (as shown in Table 15);
b. VH and VL, wherein the VH comprises the HVR-H1, HVR-H2, and HVR-H3 of the antibody CD-02 (as shown in Table 14) and the VL comprises the HVR-L1, HVR-L2, and HVR-L3 of the antibody CD-01 (as shown in Table 15);
c. VH and VL, wherein the VH comprises the HVR-H1, HVR-H2, and HVR-H3 of the antibody CD-03 (as shown in Table 14) and the VL comprises the HVR-L1, HVR-L2, and HVR-L3 of the antibody CD-01 (as shown in Table 15);
d. VH and VL, wherein the VH comprises the HVR-H1, HVR-H2, and HVR-H3 of the antibody CD-04 (as shown in Table 14) and the VL comprises the HVR-L1, HVR-L2, and HVR-L3 of the antibody CD-01 (as shown in Table 15);
e. VH and VL, wherein the VH comprises the HVR-H1, HVR-H2, and HVR-H3 of the antibody CD-05 (as shown in Table 14) and the VL comprises the HVR-L1, HVR-L2, and HVR-L3 of the antibody CD-01 (as shown in Table 15);
f. VH and VL, wherein the VH comprises the HVR-H1, HVR-H2, and HVR-H3 of the antibody CD-06 (as shown in Table 14) and the VL comprises the HVR-L1, HVR-L2, and HVR-L3 of the antibody CD-01 (as shown in Table 15);
g. VH and VL, wherein the VH comprises the HVR-H1, HVR-H2, and HVR-H3 of the antibody CD-07 (as shown in Table 14) and the VL comprises the HVR-L1, HVR-L2, and HVR-L3 of the antibody CD-01 (as shown in Table 15);
h. VH and VL, wherein the VH comprises the HVR-H1, HVR-H2, and HVR-H3 of the antibody CD-08 (as shown in Table 14) and the VL comprises the HVR-L1, HVR-L2, and HVR-L3 of the antibody CD-01 (as shown in Table 15);
i. VH and VL, wherein the VH comprises the HVR-H1, HVR-H2, and HVR-H3 of the antibody CD-09 (as shown in Table 14) and the VL comprises the HVR-L1, HVR-L2, and HVR-L3 of the antibody CD-01 (as shown in Table 15);
j. VH and VL, wherein the VH comprises the HVR-H1, HVR-H2, and HVR-H3 of the antibody CD-10 (as shown in Table 14) and the VL comprises the HVR-L1, HVR-L2, and HVR-L3 of the antibody CD-01 (as shown in Table 15);
k. VH and VL, wherein the VH comprises the HVR-H1, HVR-H2, and HVR-H3 of the antibody CD-11 (as shown in Table 14) and the VL comprises the HVR-L1, HVR-L2, and HVR-L3 of the antibody CD-01 (as shown in Table 15);
l. VH and VL, wherein the VH comprises the HVR-H1, HVR-H2, and HVR-H3 of the antibody CD-12 (as shown in Table 14) and the VL comprises the HVR-L1, HVR-L2, and HVR-L3 of the antibody CD-01 (as shown in Table 15);
m. VH and VL, wherein the VH comprises the HVR-H1, HVR-H2, and HVR-H3 of the antibody CD-13 (as shown in Table 14) and the VL comprises the HVR-L1, HVR-L2, and HVR-L3 of the antibody CD-01 (as shown in Table 15);
n. VH and VL, wherein the VH comprises the HVR-H1, HVR-H2, and HVR-H3 of the antibody CD-14 (as shown in Table 14) and the VL comprises the HVR-L1, HVR-L2, and HVR-L3 of the antibody CD-01 (as shown in Table 15);
o. VH and VL, wherein the VH comprises the HVR-H1, HVR-H2, and HVR-H3 of the antibody CD-15 (as shown in Table 14) and the VL comprises the HVR-L1, HVR-L2, and HVR-L3 of the antibody CD-01 (as shown in Table 15);
p. VH and VL, wherein the VH comprises the HVR-H1, HVR-H2, and HVR-H3 of the antibody CD-16 (as shown in Table 14) and the VL comprises the HVR-L1, HVR-L2, and HVR-L3 of the antibody CD-01 (as shown in Table 15);
q. VH and VL, wherein the VH comprises the HVR-H1, HVR-H2, and HVR-H3 of the antibody CD-17 (as shown in Table 14) and the VL comprises the HVR-L1, HVR-L2, and HVR-L3 of the antibody CD-01 (as shown in Table 15); or
r. VH and VL, wherein the VH comprises the HVR-H1, HVR-H2, and HVR-H3 of the antibody CD-18 (as shown in Table 14) and the VL comprises the HVR-L1, HVR-L2, and HVR-L3 of the antibody CD-01 (as shown in Table 15).

19. The antibody of any one of claims 1-18, wherein the antibody has one or more of the following properties:
a. the antibody increases the activity of human CD300LB and/or mouse CD300LB;
b. the antibody specifically binds to both human CD300LB and cynomolgus monkey CD300LB;
c. the antibody does not bind to murine CD300LB;
d. the antibody binds to to human CD300LB on the surface of cells overexpressing human CD300LB with a K_{D} of less than 1 μM, less than 100 nM, less than 10 nM, less than 1 nM;
e. the antibody has a higher affinity for human CD300LB than to human CD300C, human CD300D, human CD300E, and/or human CD300F;
f. the antibody does not bind to human CD300C, human CD300D, human CD300E, human CD300F, mouse CD300D, and/or mouse CD300F;
g. the antibody activates plate-bound CD300LB, for example expressed in a mouse BaF/3 cell line;
h. the antibody binds to human monocytes and human macrophages;
i. the antibody increases Syk phosphorylation in primary human macrophages; and
j. the antibody induces DAP12 tyrosine phosphorylation in wild-type mouse bone marrow derived macrophages.

20. The antibody of any one of claims 1-19, wherein the antibody is a monoclonal antibody.

21. The antibody of any one of claims 1-20, wherein the antibody is a humanized antibody.

22. The antibody of any one of claims 1-21, wherein the antibody is an antigen binding fragment, such as an Fab, Fab′, Fab′-SH, F(ab′)₂, Fv, or scFv fragment.

23. The antibody of any one of claims 1-22, wherein the antibody is a bispecific or multispecific antibody.

24. The antibody of any one of claims 1-23, wherein the antibody is of the IgG class, the IgM class, or the IgA class.

25. The antibody of claim 24, wherein the antibody is of the IgG class and is of a human IgG1, IgG2, IgG3, or IgG4 isotype or of a mouse IgG1 or IgG2 isotype.

26. The antibody of any one of claims 1-25, wherein the antibody binds to an inhibitory Fc receptor.

27. The antibody of claim 26, wherein the inhibitory Fc receptor is inhibitory Fc-gamma receptor IIB (FcgRIIB).

28. The antibody of claim 27, wherein the antibody decreases cellular levels of FcgRIIB.

29. The antibody of any one of claims 1-28, wherein the anti-CD300LB antibody has a human or mouse IgG1 isotype and comprises one or more amino acid substitutions in the Fc region at an amino acid residue selected from the group consisting of N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, N325S, T394D, A330S, E430G, E430S, E430F, E430T, E345K, E345Q, E345R, E345Y, S440Y, S440W, and any combination thereof, wherein the numbering of the residues is according to EU numbering.

30. The antibody of any one of claims 1-28, wherein the antibody has a human or mouse IgG2 isotype and comprises one or more amino acid substitutions in the Fc region at an amino acid residue selected from the group consisting of A330S, C127S, C214S, C219S, C220S, E345K, E345Q, E345R, E345Y, E430F, E430G, E430S, E430T, G237A, H268Q, L328F, M252Y, P331S, S254T, S267E, S440W, S440Y, T256E, V234A, V309L, and any combination thereof, wherein the numbering of the residues is according to EU numbering.

31. The antibody of any one of claims 1-28, wherein the anti-CD300LB antibody has a human or mouse IgG4 isotype and comprises one or more amino acid substitutions in the Fc region at an amino acid residue selected from the group consisting of C127S, E318A, E345R, E430G, F234A, G237A, K322A, L235A, L235E, L236E, L243A, L328F, M252Y, P331S, S228P, S229P, S254T, S267E, S440Y, T256E, and any combination thereof, wherein the numbering of the residues is according to EU numbering.

32. The antibody of any one of claims 1-31, wherein the antibody comprises one or more amino acid substitutions in the Fc region at a residue position selected from the group consisting of A330L, A330S, C127S, E345R, E430G, K322A, L234A, L234F, L235A, L235E, L243A, L328F, P331S, S267E, S440Y, and any combination thereof, wherein the numbering of the amino acid residues is according to EU or Kabat numbering.

33. A pharmaceutical composition comprising the anti-CD300LB antibody of any one of claims 1-32 and a pharmaceutically acceptable carrier.

34. An isolated nucleic acid comprising a nucleic acid sequence encoding the anti-CD300LB antibody of any one of claims 1-32.

35. An isolated vector comprising the nucleic acid of claim 34.

36. An isolated host cell comprising the nucleic acid of claim 34 or the vector of claim 35.

37. A method of producing an antibody that binds to human CD300LB, comprising culturing the cell of claim 36 so that the antibody is produced.

38. The method of claim 37, further comprising recovering the antibody produced by the cell.

39. A method of treating a neurodegenerative disease or disorder, the method comprising administering to an individual in need thereof a therapeutically effective amount of an anti-CD300LB antibody of any one of claims 1-32, thereby treating the disease or disorder.

40. The method of claim 39, wherein the disease or disorder is Alzheimer's disease.

41. A method of detecting the presence of CD300LB in a sample or an individual, the method comprising an anti-CD300LB antibody of any one of claims 1-32.

42. The method of claim 41, further comprising quantification of antigen-bound anti-CD300LB antibody.
